(19) United States
(12) Patent Application Publication
(10) Pub. No.: US 2006/0241284 A1
(43) Pub. Date: Oct. 26, 2006

(54) Transmembrane protein amigo and uses thereof

(76) Inventors: Juha Kuja-Panula, Helsinki (FI); Marjaana Kiiltomaki, Helsinki (FI); Heikki Rauvala, Helsinki (FI)

Correspondence Address: BIRCH STEWART KOLASCH & BIRCH, PO BOX 747, FALLS CHURCH, VA, 22040-0747, US

(21) Appl. No.: 10/537,102

(22) PCT Filed: Dec. 12, 2003
(86) PCT No.: PCT/FI03/00949

§ 371 Date: Jun. 2, 2005

Related U.S. Application Data
(60) Provisional application No. 60/433,011, filed on Dec. 13, 2002.

(51) Int. Cl. C07K 14/705; C07K 16/28; C12Q 1/68; C07H 21/04; C12P 21/06

(52) U.S. Cl. 530/350; 530/388.22; 435/6; 435/69.1; 435/320.1; 435/325; 536/23.5

(57) ABSTRACT

The present invention provides methods and compositions relating to vertebrate AMIGO, AMIGO2, AMIGO3, collectively vertebrate AMIGO polypeptides, related nucleic acids, and polypeptide domains thereof having vertebrate AMIGO-specific structure and activity, and modulators of vertebrate AMIGO function.

### FIELD OF THE INVENTION

The present invention generally relates to the field of genetic engineering and more particularly to transmembrane proteins implicated in axon tract development.

### BACKGROUND OF THE INVENTION

Development of the nervous system with billions of connections is one of the most complex and fascinating phenomena in nature. One key feature in this event is the guidance of the neuronal growth cones to their appropriate targets. A wide variety of soluble matrix and cell surface molecules have been found to be involved in axonal growth and in association of axons to form mature fiber tracts (for reviews, see Tessier-Lavigne and Goodman, 1996; Drescher et al., 1997).

Within the peripheral nervous system (PNS), injured nerve fibers can regrow over long distances, with eventual excellent recovery of function. Within the past 15 years, neuroscientists have come to realize that this is not a consequence of intrinsic differences between the nerve cells of the peripheral and central nervous system; remarkably, neurons of the CNS will extend their axons over great distances if given the opportunity to grow through a grafted segment of PNS (e.g., sciatic nerve). Therefore, neurons of the CNS retain a capacity to grow if given the right signals from the extracellular environment. Factors which contribute to the differing growth potentials of the CNS and PNS include partially characterized, growth-inhibiting molecules on the surface of the oligodendrocytes that surround nerve fibers in the CNS, but which are less abundant in the comparable cell population of the PNS (Schwann cells); molecules of the basal lamina and other surfaces that foster growth in the PNS but which are absent in the CNS (e.g., laminin); and trophic factors, soluble polypeptides which activate programs of gene expression that underlie cell survival and differentiation. Although such trophic factors are regarded as essential for maintaining the viability and differentiation of nerve cells, the particular ones that are responsible for inducing axonal regeneration in the CNS remain uncertain. As a result, to date, effective treatments for CNS injuries have not been developed.

Immunoglobulin superfamily proteins form the most diverse and studied class of molecules, which have been shown to participate in contact-dependent regulation of neurite outgrowth, axon guidance and synaptic plasticity (for reviews see, Schachner; 1997; Walsh and Doherty, 1997; Stoeckli and Landmesser, 1998; Van Vactor, 1998). Extracellular proteins containing leucine-rich repeats (LRRs) have also been shown to participate in axon guidance. For instance, Slit proteins containing LRR domains act as midline repellents for commissural axons through the Robo (Roundabout) receptor (Battye et al., 1999; Brose et al., 1999) and recently Battye et al. (2001) showed that the interaction of Slits with their Robo receptors was due to LRRs found in Slits. Furthermore, Pusch et al. (2000) showed that the disease called X-linked congenital stationary night blindness (XLCSNB) maps to a gene, which codes only the LRR containing protein Nyctalopin in retina. Recently the receptor for axonal regeneration inhibitor Nogo (Chen et al., 2000) was found to be a GPI-linked cell surface protein where the only recognizable motifs are LRR domains (Fournier et al., 2001).

Amphoterin (also known as HMGB1) is a heparin-binding protein that was isolated from perinatal rat brain as a neurite outgrowth-promoting factor (Rauvala and Pihlaskari, 1987) enriched in the growth cones of neuronal cells. Amphoterin has been proposed to be an autocrine factor in invasive cell or growth cone migration due to binding to the cell surface receptors (RAGE and sulphated glycan epitopes) and to activation of proteolysis of ECM through binding of plasminogen and it's activators to amphoterin (for reviews see Rauvala et al., 2000; Muller et al., 2001).

To examine the role of amphoterin in cell motility, especially in neurite outgrowth, we searched for genes that are induced on amphoterin matrix by using mRNA differential display. In this invention, we describe the cloning and functional characterization of a novel protein named as AMIGO (AMphoterin Induced Gene and Orphan receptor). Cloning of AMIGO gave us sequence data to clone two other related proteins (AMIGO2 and AMIGO3); together these three proteins form a novel family of transmembrane proteins. The predicted amino acid sequences of the AMIGOs suggest that they are type I transmembrane proteins containing a signal sequence for secretion and a transmembrane domain. Interestingly, the extracellular part of the AMIGOs contains six leucine-rich repeats (LRRs) flanked by cysteine-rich LRR N- and C-terminal domains and one immunoglobulin domain close to the transmembrane region. This twin motif structure defines the AMIGOs as members of both the immunoglobulin and the leucine-rich repeat superfamilies.

Amphoterin

Amphoterin is a protein, which was isolated from perinatal rat brain according to its ability to promote neurite outgrowth (Rauvala and Pihlaskari, 1997). Amphoterin is a dipolarised molecule, which contains both positively and negatively charged regions. This dipolar nature of amphoterin renders it very adhesive molecule, which binds for example to heparin and other sulphated glycans.

Amphoterin is also found to localize in nucleus and to bind DNA and in this role it is called as HMG1 (Bianchi et al., 1989). In subsequent studies amphoterin has been shown to localize diffusibly inside the cell but when the cell starts to grow projections amphoterin is localized into the tips of the projections (Merenmies et al., 1991; Parkkinen et al., 1993). Although amphoterin lacks the signal sequence for secretion, it has been shown to be present also in the extracellular matrix (ECM). In vitro amphoterin has been shown to localize to the surface of the neurons (Rauvala and Pihlaskari, 1997; Rauvala et al., 1988) and amphoterin has been shown to be a ligand for the cell surface receptor RAGE (Hori et al., 1995). During the endotoxin shock large quantities of amphoterin has been shown to accumulate into the human plasma (Wang et al., 1999). During the period when the red blood cells are maturing amphoterin is secreted into the ECM, where it is believed to work as a differentiation factor (Passalacqua et al., 1997). It is also suggested that the amphoterin secreted from glial cells works as a factor between the interaction of glia and neurons (Passalacqua et al., 1998; Daston and Ratner, 1994).

Amphoterin is highly expressed in neurons and glial cells in developing nervous system and generally in non-mature cells. Amphoterin is also highly expressed in monocytes and macrophages and often in transformed cells. Amphoterin is thought to be involved in invasive migration of cells. Amphoterin binds plasminogen and plasminogen activators and this binding has been shown to activate the formation of the plasmin and also degradation of amphoterin (Parkkinen and Rauvala, 1991; Parkkinen et al., 1993). At the cell surface level amphoterin binds to the transmembrane protein RAGE and some proteoglycans (like Syndecan-1) and sulphoglycolipids. The multiligand protein RAGE (Receptor of advanced glycation end products) is a member of immunoglobulin superfamily. Amphoterin stimulates the neurite outgrowth via RAGE dependent signalling and the both proteins also localize in same areas of the developing nervous system (Hori et al., 1995). It has been suggested that amphoterin works as an autocrine and/or paracrine factor in invasive migration; amphoterin binds to its receptors and activates both the proteolysis of the ECM and the reorganization of the cytoskeleton (Rauvala et al., 2000; Rauvala et al., 1988). It has been shown that by inhibiting the interaction between amphoterin and RAGE the growth and the invasiveness of the tumour could be reduced.

Immunoglobulin Domains

IgG domain is one of the most common extracellular protein motifs. It was first discovered from antibody molecules. In addition of antibody molecules, many cell adhesion molecules, cell surface receptors and some intracellular muscular proteins contain IgG domains. IgG domain is about 70-110 amino acids long usually containing two cysteines separated by 55-77 amino acids, it forms 7-10 beta sheets, and is a tightly packed globular structure with hydrophobic residues inside and hydrophilic outside. The structure is often stabilized by disulfide bridge between conserved cysteines (Walsh and Doherty 1997; Williams and Barclay 1988).

In sequence level IgG domains differ greatly. The homology between different IgG domains within the same protein may share only 10-30% amino acid similarity. Although all IgG domains share the same core structure, two beta sheets stacked together, the other features can vary considerably. In spite of variability within IgG domains they can be classified in categories. Originally they were classified as C1, C2 and V, and later Group I was added (Williams and Barclay 1988). The stability of IgG domain may explain why it commonly resides in extracellular space, it is resistant to proteolytic and oxidative environment. Extracellular IgG domain containing molecules may function in cell adhesion and in recognition and binding of molecules. IgG domain seems to interact with any parts of its domain surface. (Williams and Barclay 1988)

IgG domain containing proteins form so called immunoglobuling superfamily of proteins, which is the most common family of cell surface proteins. Sequence analysis has shown that 765 human proteins belong to this family, in flyes there are 140 and in worms 64 proteins (Venter et al 2001). The members of IgG family encode proteins that are involved in cell recognition and adhesion such as antibody molecules, T-cell receptors, growth factor receptors, many adhesion molecules and neurite outgrowth promoting receptors. IgG domain adhesion molecules often consist of several consequent IgG domains and type III fibronectin like domains (Crossin and Krushel 2000).

Neuronal members of immunoglobuling superfamily act as receptor and adhesion molecules and their role have been especially indicated in many important functions related to axonal growth and guidance. Adhesion molecules have important roles during the neuronal development where many interactions need to be coordinated in precise manner, for example NCAM and L1 which function during axonal growth and guidance (Walsh and Doherty 1997). Other members include receptor for FGF (FGFR, Trk family of neurotrophic factor receptors, Eph receptors, Robo (Roundabout) that mediates the functions of Slit and DCC (Deleted in colorectal carcinoma) that interacts with netrins (Tessier-Lavigne and Goodman 1996; Brose and Tessier-Lavigne 2000).

Axonal IgG cell adhesion molecules may interact in homophilic or heterophilic way with other IgG family members. The binding partner may localize at the same cell membrane, in adjacent cell membrane or in extracellular space. Many IgG proteins form a very complex network of cellular interactions where they can even have partially overlapping functions. They may also compete for the same ligands by modulating their binding affinity to other ligands (Brummendorf and Lemmon 2001).

Immunoglobulin superfamily members involved in myelinization are MAG (myelin-associated glycoprotein) and P0 although their precise actions are not known. MAG's functions have been shown to be associated with inhibition of regeneration of CNS neurons or it can either activate or inhibit the neurite growth of certain neurons. Approximately half of the all protein in myelin consists of P0 protein which is a homophilic cell adhesion molecule thought to be involved in interconnection of cell membranes of myelin sheath (Brummendorf and Rathjen 1994).

LRR Domains

Leucine rich repeats (LRR) are 20-29 amino acid long sequence motifs characterized by repetition of hydrophobic residues, especially leucine and that are separated by conserved distance. The sequence repeat is found in several times in protein and this region of repeats is called LRR domain. LRR contains conserved 11 amino acid long consensus sequence, LxxLxLxxzxL where x stands for any amino acid, z for N or cysteine and L for leucine, valine, isoleucine or phenylalanine. LRR proteins contain usually many LRR domains and can contain up to 30 repeats (chaoptin). LRR domains are not always identical to consensus sequence and may therefore contain gaps, have different lengths or amino acid compositions (Kobe and Deisenhofer 1994).

To prevent the sole hydrophobic core of LRR domain from interacting directly with solvents it is flanked by several cysteine residues at its N and/or C terminal sides (LRRNT, LRRCT domains). Sequence analysis has revealed that there are four different C terminal cysteine rich domains and one N terminal one (Kobe and Kajava 2001; Kajava 1998). These cysteine domains are only found from extracellular proteins and cysteines form intermolecular disulfide bridges (Kresse et al 1993; Hashimoto et al 1991).

LRR domain proteins are located in various places in cells and they have different functions. Eukaryotic LRR proteins can be found in nucleus, cytoplasm, cell membrane as well as in extracellular space and they can act as hormone receptors, subunits of enzymes, cell adhesion molecules and in cell recognition (Kobe and Kajava 2001). Moreover, they mediate various cellular functions such as signal transduction, intracellular transport, and DNA repair, recombination and transcription (Buchanan and Gay 1996).

LRR proteins can be divided into at least 7 different subclasses according to the length of LRR and the composition of consensus sequence. Subclasses are RI-like, SDS22-like, cysteine containing, bacterial, typical, plant specific and TpLRR (Kobe and Kajava 2001). Former three are intracellular whereas latter four are found in cellular membranes or in the extracellular space.

LRR domains are thought to have a role in protein-protein interactions. For example, chaoptin is a cell surface protein which consists of LRR domains, is attached to cell membrane via lipid anchor, and has been shown to mediate homophilic cell adhesion (Reinke et al 1988; Krantz and Zipursky 1990).

Extracellular matrix contains several homologous small proteglycans whose sequences are composed 70-80% of LRR domains. These small proteoglycans are composed of N-terminal glycosaminoglycans and variable amounts of LRRs that are flanked by LRRNT and LRRCT domains. Proteoglycans such as biglycan binds to laminin and fibronectin whereas decorin and fibromodulin bind to type I and II collagens (Svensson et al 1995). Axonal growth modulating molecule Slit contains LRR domain, EGF repeats, laminin like G domain and LRRNT and LRRCT domains. Only LRR domain of Slit is needed for its binding to Robo in vitro as well as mediating repulsive signalling in vivo (Battye et al 2001).

Although several LRR proteins are expressed in the nervous system only few of those functions or binding partners are known. The best characterized neuronal LRR proteins are Drosophila's connectin, capricious and chaoptin. Connectin is a GPI (glycosyl phosphatidyl inositol) linked cell adhesion protein that has a role during the development of neuromuscular junction. It contains 10 LRR domains flanked by LRRCT domain. During the formation of neuromuscular junction connectin is expressed in surfaces of certain muscle cells and concomitantly in their innervating motor neurons where the expression is especially seen in growth cones. During synapse formation connectin localizes in junctional areas but during synapse maturation connectin expression is downregulated. In vitro experiments have indicated increased homophilic cell adhesion between connectin transfected S2 cells (Nose et al 1992; Meadows et al 1994). Moreover, in vivo studies have supported its role as attractive neuronal growth modulating protein. When connectin is misexpressed in all muscular cells aberrant neuromuscular junction formation occurs (Yu et al 2000).

Capricious is a cell membrane protein sharing similarities with the functions of connectin in neuromuscular junction formation. It contains 12 LRR domains flanked by LRRCT and LRRNT domains. It probably mediates cell-to-cell signalling processes during the formation of the neuromuscular junction since in vitro studies have not supported its homophilic adhesion (Shishido et al 1998).

Chaoptin is a photoreceptor cell specific adhesion molecule which contains 30-40 LRR domains and is linked to cell membrane via GPI anchor. It mediates homophilic cell adhesion and is needed for the proper formation of a photoreceptor cell (Krantz and Zipursky 1990).

Slit proteins are conserved, secreted into extracellular space and provide guidance during axonal growth and branching. Slit proteins consist of several LRR domains, EGF like repeats, laminin like G-domain and LRRNT domain (Brose and Tessier-Lavigne 2000). Slit was discovered from fruit fly where it repels axonal growth (Rothberg et al 1990; Kidd et al 1999). Slit is produced by glial cells of midline and it is needed for the formation of axonal tracts crossing the midline as well as positioning of horizontal lateral tracts. Biological functions of Slit are mediated by Robo which is a cell membrane receptor. LRR domains of Slit bind to Robo in vitro and LRR domains are needed for Slit's repulsive signalling (Battye et al 2001). Three mammalian Slits and Robos have been cloned. In addition, Slit binds to laminin-1, netrin-1, and glypican-1 (Brose et al 1999; Liang et al 1999).

Nogo receptor (NogoR) is CNS receptor protein found in myelin and responsible for the inhibition of axonal regeneration. NogoR consists of 8 LRR domains flanked by LRRCT domain and it is attached to cell membrane via GPI anchor. It binds to Nogo-66 while inhibiting axonal growth (Grandpre and Strittmatter 2001; Fournier et al 2001).

OMgp is oligodendrocyte-myelin glycoprotein found in CNS myelin and cell membranes of oligodendrocytes. It is 110 kDa GPI-anchored cell membrane protein containing at least 6 LRR domains and LRRNT domain (Mikol et al 1988 and 1990). Like Nogo, OMgp inhibits axonal regeneration in the mammalian CNS. Until recently OMgp has been shown to bind NogoR while inhibiting axonal regeneration (Wang et al 2002).

LRR- and Ig-Domains Containing Proteins

Some transmembrane proteins of nervous system contain both LRR- and Ig-domains, which are discussed below.

Kekkon and ISLR

Drosophilae (fruit fly) has gene family called kekkon, which codes transmembrane proteins with both LRR- and Ig-domains. The extracellular part of the kekkon1 (kek1) and kekkon2 (kek2) contains six LRRs flanked with LRRNT and LRRCT domains. They also contain one type C2 Ig-domain close to transmembrane region and large intracellular tail. Both genes are expressed in developing central nervous system (CNS) and the kekkon1 is also present in developing ovary (Musacchio and Perrimon, 1996). The kek1 has been shown to inhibit the function of the epidermal growth factor receptor (EGFR) in oogenesis (Ghiglione, 1999). Interestingly, only the extracellular part and transmembrane domains of the kek1 protein are needed for EGFR inhibition.

The transmembrane protein ISLR has same kind of domain structure as the kekkon proteins. The extracellular part of the ISLR contains six LRRs lined with LRRNT and LRRCT domains. It also contains one type C2 Ig-domain close to transmembrane region but it does not contain intracellular part. The ISLR has been cloned from humans and mice. The ISLR is expressed in various tissues like retina, heart, thymus and spinal cord (Nagasawa et al., 1999; Nagasawa et al., 1997).

Trk-Receptors

Neurotrophin receptors TrkA, TrkB and TrkC are receptor tyrosine kinases, in which the extracellular part contains three LRR-areas and each area is flanked with LRRNT and LRRCT domains. The extracellular part contains also two Ig-domains. The intracellular parts of Trk-receptors contain tyrosine kinase domain. The ligands of Trk-receptors are neurotrophins, which are important factors in development and in maintenance of central and peripheral nervous system. The binding of the neurotrophins into the Trk-receptor dimerazes the receptor and the tyrosine kinase domain is autophosphorylated and this phosphorylation activates several signalling cascades (Kaplan and Miller, 1997). Originally, some studies indicated that the LRR-areas of the Trk-receptors are the ligand binding domains (Windisch et al., 1995; Windish et al., 1995). Recently it has been shown that the Ig-domain of the TrkA receptor closest to the cell surface is the one, which binds the Nerve growth factor (NGF)(Holden et al., 1997; Perez et al., 1995; Robertson et al., 2001; Urfer et al., 1995; Wiesmann et al., 1999).

NLRRs, Pal and LIG-1

Neuronal Leucine-rich repeat proteins (NLRRs) are transmembrane proteins expressed in nervous tissues. The extracellular part of the NLRRs contains 12 LRRs flanked with LRRNT and LRRCT domains, one Ig-domain and type III fibronectin like domain. Similar NLRR proteins have been found from mouse, rat, zebra fish, frog and human (Hayata et al., 1998; Taniguchi et al., 1996: Taguchi et al., 1996; Bormann et al., 1999; Fukamachi et al., 1998). In zebra fish one member of NLRR family is expressed specifically during the axonal regeneration after injury (Hayata et al., 1998). Unlike in adult mammalian CNS the neurons of the fish could raise new neurons into the injured area. In mouse NLRR-3 gene has been shown to be induced after cortical injury (Ishii et al., 1996).

Pal is a transmembrane protein, which is expressed specifically in retina. The extracellular part of the pal contains five LRRs flanked with LRRNT and LRRCT domains, one type C2 Ig-domain and type III fibronectin like domain. In adult retina pal is expressed by photoreceptor cells, where protein is believed to localize in disks. The function of the pal is not yet known, but it has been shown to form homodimers (Gomi et al., 2000).

LIG-1 is also a transmembrane protein, which contains both LRRs and immuoglobulin domains. The extracellular part of the LIG-1 contains 15 LRRs and three type C2 Ig-domains. The intracellular part of the LIG-1 is 270 amino acids long and it does not contain any known domains. LIG-1 is expressed highly in brain both in mice and humans. In mouse the LIG-1 expression is localized in particular subpopulations of neuronal support cells; in cerebellum LIG-1 is localized in Bergman glia cells (Nilsson et al., 2001; Suzuki et al., 1996).

In this invention we have characterized AMIGO, AMIGO2 and AMIGO3, the members of the protein family that is highly expressed in the nervous system. We disclose that AMIGOs mediate cell-to-cell interactions via a homophilic and heterophilic mechanism during the development of the fiber tracts of the nervous system.

Epidermal Growth Factor Receptor

Epidermal growth factor receptor (EGFR) is a 170 kDa transmembrane glycoprotein which possesses the intrinsic tyrosine kinase activity (Cohen et al., 1982). EGFR exerts a great variety of biological functions including cell survival, mitogenic response, differentiation and cell motility (Khazaie et al., 1993). Many ligands for EGFR have been identified including epidermal growth factor (EGF), transforming growth factor alpha (TGF-α), amphiregulin (AR), epiregulin (EP), Batacellulin (BTC), Heparin-binding EGF-like growth factor (HB-EGF) and Schwannoma-derived growth factor (SDGF). The EGF-family of peptides is significantly involved in the regulation of mammary-gland development, morphogenesis and lactation, and also implicated in the pathogenesis of human breast cancer (Normanno and Ciardiello, 1997).

Epidermal Growth Factor Receptor (EGFR) (SEQ ID NOS:21-24) is a specific receptor for epidermal growth factor (EGF) (SEQ ID NOS:25-28) and transforming growth factor-α (TGF-α) (SEQ ID NOS:29-32). When these mitogenic polypeptides bind to EGFR, tyrosine kinase activity of the receptor is induced, and this in turn triggers a series of events which regulate cell growth. A number of malignant and non-malignant disease conditions are now believed to be associated with EGFR, particularly aberrant expression of EGFR. Aberrant expression includes both increased expression of normal EGFR and expression of mutant EGFR. Overexpression of EGFR is found in many human tumors including most glioblastomas and breast, lung, ovarian, colorectal, bladder, pancreatic, squamous cell and renal carcinomas. Elevated EGFR levels correlate with poor prognosis in human tumors. The sequence of the mRNA encoding human EGFR is known (Ullrich et al., Nature, 1984, 309, 418; GenBank Accession Number NM_{—}005228). The gene encoding EGFR is also known as c-erb-B1. Two EGFR transcripts typically appear on Northern blots, one measuring 10 kb and one measuring 5.6 kb.

One role the EGF receptor system may play in the oncogenic growth of cells is through autocrine-stimulated growth. If cells express the EGFR and secrete EGF and/or TGF-α then such cells could stimulate their own growth. Since some human breast cancer cell lines and tumors express EGFR (Osborne, et al., J. Clin. Endo. Metab., 55:86-93 (1982); Fitzpatrick, et al., Cancer Res., 44:3442-3447 (1984); Filmus, et al., Biochem. Biophys. Res. Commun., 128:898-905 (1985); Davidson, et al., Mol. Endocrinol., 1:216-223 (1987); Sainsbury, et al., Lancet, i: 1398-1402 (1987); Perez, et al., Cancer Res. Treat., 4:189-193 (1984)) and secrete TGF.alpha. A (Bates, et al., Cancer Res., 46:1707-1713 (1986); Bates, et al., Mol. Endocrinol., 2:543-555 (1988)), an autocrine growth stimulatory pathway has been proposed in breast cancer (Lippman, et al., Breast Cancer Res. Treat., 7:59-70 (1986)).

A number of inhibitors of EGFR have been shown to be effective in inhibiting the growth of human tumor cells. Monoclonal antibodies to EGFR and drugs which inhibit EGFR tyrosine kinase activity can inhibit the growth of human cancer cell xenografts in nude mice. Normanno et al., Clin. Cancer Res., 1996, 2, 601 and Grünwald et al, J Nat Cancer Inst, 2003, 95:851. The drug PD153035, which inhibits EGFR tyrosine kinase activity, can inhibit the growth of A431 cells in nude mice, and tyrphostins, which inhibit the activity of EGFR as well as other tyrosine kinases, have been shown to inhibit the growth of squamous carcinoma in nude mice. Kunkel et al., Invest. New Drugs, 1996, 13, 295 and Yoneda et al., Cancer Res., 1991, 51, 4430. Additonal small molecule tyrosine kinase inhibitors include ZD1839, OSI-774, CI-1033, PKI-166, GW2016, EKB-569, PD168393, AG-1478, and CGP-59326A (Grünwald et al, J Nat Cancer Inst, 2003, 95:851 incorporated herein by reference in the entirety.

Furthermore, EGFR expression is frequently accompanied by the production of EGFR-ligands, TGF-α and EGF among others, by EGFR-expressing tumor cells which suggests that an autocrine loop participates in the progression of these cells (Baselga, et al. (1994) Pharmac. Therapeut. 64:127-154; Modjtahedi, et al. (1994) Int. J. Oncology. 4:277-296). Blocking the interaction between such EGFR ligands and EGFR therefore can inhibit tumor growth and survival (Baselga, et al. (1994) Pharmac. Therapeut. 64:127-154).

A variety of approaches can be used to target EGFR such as using monoclonal antibodies to compete with the binding of activating ligands to the extracellular domain of the receptor, using small molecule inhibitors ot the intracellular tyrosine kinase domain of the receptor, using immunotoxin conjugates to deliver toxins that target EGFR to tumour cells, reducing the level of EGFR through the use of antisense oligonucleotides, and inhibiting downstream effectors of the EGFR signalling network. Despite the foregoing approaches, the need exists for new compounds against EGFR which are effective at treating and/or preventing diseases related to expression of EGFR.

### SUMMARY OF THE INVENTION

The present invention provides methods and compositions relating to vertebrate AMIGO, AMIGO2, AMIGO3, collectively vertebrate AMIGO polypeptides, related nucleic acids, and polypeptide domains thereof having vertebrate AMIGO-specific structure and activity, and modulators of vertebrate AMIGO function. Vertebrate AMIGO polypeptides can regulate cell, especially nerve cell, function and morphology. The polypeptides may be produced recombinantly from transformed host cells from the subject vertebrate AMIGO polypeptide encoding nucleic acids or purified from mammalian cells. The invention provides isolated vertebrate AMIGO hybridization probes and primers capable of specifically hybridizing with natural vertebrate AMIGO genes, vertebrate AMIGO-specific binding agents such as specific antibodies, and methods of making and using the subject compositions in diagnosis (e.g. genetic hybridization screens for vertebrate AMIGO transcripts), therapy (e.g. to modulate nerve cell growth) and in the biopharmaceutical industry (e.g. as immunogens, reagents for isolating vertebrate AMIGO genes and polypeptides and reagents for screening chemical libraries for lead pharmacological agents.

In one embodiment, the invention contemplates in vitro methods and kits for culturing neuronal cells under conditions where the subject polypeptides are used to promote neurite outgrowth, and can include methods for detecting the presence and amount of stimulation of neurite outgrowth in the cultured neuronal cells. AMIGO proteins and polypeptides disclosed herein are useful according to the within-disclosed methods and may be included in the kits that are also described herein.

Appropriate cells are prepared for use in a neurite outgrowth assay. For example, a preparation of hippocampal neurons is disclosed in the Examples. Before beginning the assay, the cells may be resuspended, added to substrate-coated dishes, and placed under predetermined assay conditions for a preselected period of time. After the attachment and growth period, the dishes may be rinsed to remove unbound cells, fixed, and viewed—e.g., by phase contrast microscopy.

Preferably, a plurality of cells are analyzed for each substrate. Cells are then “judged” based on predetermined criteria. For example, cells may be considered neurite-bearing if the length of the processes are greater than one cell diameter. The percent of cells that are sprouting neurites is preferably determined, as is the average neurite length. A particularly preferred neurite outgrowth assay method is disclosed in the Examples.

The proteins and polypeptides of the present invention are therefore useful in a variety of applications relating to cell and tissue cultures.

For example, in one embodiment, a method of inhibiting neurite outgrowth of neuronal cells in a cell culture system comprises the steps of (1) introducing neuronal cells into tissue culturing conditions comprising a culture medium; and (2) introducing an AMIGO polypeptide of the present invention into the culture medium in an amount effective to inhibit neurite outgrowth in the culture.

In another embodiment, a method of promoting neurite outgrowth of neuronal cells in a cell culture system comprises the steps of (1) immobilizing on the substrate a polypeptide of the present invention having neurite outgrowth-promoting activity; and (2) contacting neuronal cells with the substrate under tissue culturing conditions.

In another embodiment, a method of promoting neurite outgrowth of neuronal cells in a cell culture system comprises the steps of (1) introducing an AMIGO nucleic acid encoding peptide having neurite outgrowth-promoting activity of the present invention; (2) immobilizing on the substrate a polypeptide of the present invention having neurite outgrowth-promoting activity; and (3) culturing said neuronal cells under tissue culturing conditions.

The invention also discloses compositions comprising polypeptides exhibiting a neurite outgrowth-promoting in substantially pure form. In various embodiments, the polypeptides are derived from segments of an AMIGO protein.

In another embodiment, a composition according to the present invention comprises a subject polypeptide in substantially pure form and attached to a solid support or substrate.

The solid support may be a prosthetic device, implant, or suturing device designed to have a surface in contact with neuronal cells or the like; further, it may be designed to lessen the likelihood of immune system rejection, wherein said surface of said device is coated with a subject polypeptide or other material designed to ameliorate rejection.

The AMIGO proteins, polypeptides, and nucleid acids disclosed herein are also useful in a variety of therapeutic applications as described herein.

The present therapeutic methods are useful in treating peripheral nerve damage associated with physical or surgical trauma, infarction, toxin exposure, degenerative disease, malignant disease that affects peripheral or central neurons, or in surgical or transplantation methods in which new neuronal cells from brain, spinal cord or dorsal root ganglia are introduced and require stimulation of neurite outgrowth from the implant and innervation into the recipient tissue. Such diseases further include but are not limited to CNS lesions, gliosis, Parkinson's disease, Alzheimer's disease, neuronal degeneration, and the like. The present methods are also useful for treating any disorder which induces a gliotic response or inflammation.

In treating nerve injury, contacting a therapeutic composition of this invention with the injured nerve soon after injury is particularly important for accelerating the rate and extent of recovery.

Thus the invention contemplates a method of promoting neurite outgrowth in a subject, or in selected tissues thereof, comprising administering to the subject or the tissue a physiologically tolerable composition containing a therapeutically effective amount of a neurite outgrowth-promoting AMIGO compound of the present invention.

In preferred methods, a human patient is the subject, and the administered polypeptide comprises extracellular domain of human AMIGO. In anothere preferred method, a human patient is the subject, and the administered nucleic acid encodes AMIGO extracellular domain of human AMIGO.

In one embodiment, a severed or damaged nerve may be repaired or regenerated by surgically entubating the nerve in an entubalation device in which an effective amount of a neurite outgrowth-promoting polypeptide of this invention can be applied to the nerve.

In a related embodiment, a polypeptide of the invention can be impregnated into an implantable delivery device such as a cellulose bridge, suture, sling prosthesis or related delivery apparatus. Such a device can optionally be covered with glia, as described by Silver, et al, Science 220:1067-1069, (1983), which reference is hereby incorporated by reference.

Therapeutic compositions of the present invention may include a physiologically tolerable carrier together with at least one species of neurite outgrowth-promoting polypeptide of this invention as described herein, dispersed therein as an active ingredient. In a preferred embodiment, the therapeutic composition is not immunogenic when administered to a human patient for therapeutic purposes.

For the sake of simplicity, the active agent of the therapeutic compositions described herein shall be referred to as a “neurite outgrowth-promoting polypeptide”. It should be appreciated that this term is intended to encompass a variety of AMIGO polypeptides including fusion proteins, synthetic polypeptides, and fragments of naturally ocurring proteins, as well as derivatives thereof, as described herein. This term also encompass the nucleid acids encoding AMIGO polypeptides including fusion proteins, synthetic polypeptides, and fragments of naturally ocurring proteins, as well as derivatives thereof, as described herein.

The methods can optionally be practiced in combination with contacting the neuronal cells or nerves with other agents capable of promoting neuron survivals growth, differentiation or regeneration.

The discovery that AMIGO proteins described herein can promote neurite outgrowth, provides agents for use in improving nerve regeneration or promoting nerve survival, in treating peripheral nerve injury and spinal cord injury, and in stimulation of growth of endogenous, implanted or transplanted CNS tissue.

The present invention therefore also provides a method of promoting regeneration of an injured or severed nerve or nerve tissue, or promoting neurite outgrowth in neuronal cells under a variety of neurological conditions requiring neuronal cell outgrowth. The method comprises contacting a neuronal cell capable of extending neurites, or an injured or severed nerve, with a cell culture system comprising a substrate containing a neurite outgrowth-promoting polypeptide of this invention in an amount effective to promote neurite outgrowth. The method may be carried out in vitro or in vivo.

The polypeptides and nucleic acids used in the present method can be any of the subject polypeptides described herein.

Any of a variety of mammalian neuronal cells can be treated by the present method in the cell culture system, including neuronal cells from brain, CNS, peripheral nerves and the like. In addition, the cells can be from any of a variety of mammalian species, including human, mouse, chicken, and any other mammalian species, including the agricultural stock and non-domesticated mammals.

In selecting a particular subject polypeptide for use in the methods, any of the polypeptides described herein can be utilized to promote neurite outgrowth, irrespective of the species of neuronal cell and species of AMIGO protein from which a subject polypeptide is derived. However, it is preferred to use a human AMIGO protein to induce neurite outgrowth on a human neuronal cell, and the like species selectivity. Thus, in preferred embodiments, the method uses rat neuronal cells and a polypeptide derived from a rat AMIGO protein, or human neuronal cells and a polypeptide derived from a human AMIGO protein, or mouse neuronal cells and a polypeptide derived from a mouse AMIGO protein, etc.

The neurite outgrowth-promoting composition can be attached to the substrate, can be contacted in the liquid phase or in a collagen gel phase. Depending on the assay system used, the AMIGO protein may promote outgrowth when bound onto the solid surfaces but may inhibit the neuronal outgrowth when provided in liqued phase. The composition may contain the subject polypeptide in the form of a fusion protein as described herein. The method may be practiced using the subject polypeptide in any of the various apparati format described herein.

The invention also provides methods and compositions for identifying agents which modulate the interaction of AMIGO with AMIGO, Epiethelial Growth Factor Receptor or AMIGO ligand (AMIGO ligand may be selected from the group of binding partner, endogenous, exogenous protein or substance capable of binding to AMIGO) and for modulating these interactions. The methods for identifying AMIGO modulators find particular application in commercial drug screens. These methods generally comprise (1) combining an AMIGO polypeptide, an AMIGO, EGFR or ligand polypeptide and a candidate agent under conditions whereby, but for the presence of the agent, the AMIGO and AMIGO/EGFR/AMIGO ligand polypeptides engage in a first interaction, and (2) determining a second interaction of the AMIGO and AMIGO/EGFR/AMIGO ligand polypeptides in the presence of the agent, wherein a difference between the first and second interactions indicates that the agent modulates the interaction of the AMIGO and AMIGO/EGFR/AMIGO ligand polypeptides.

The subject methods of modulating the interaction of AMIGO involve combining an AMIGO polypeptide, an AMIGO/EGFR/AMIGO ligand polypeptide and a modulator under conditions whereby, but for the presence of the modulator, the AMIGO and AMIGO/EGFR/AMIGO ligand polypeptides engage in a first interaction, whereby the AMIGO and AMIGO/EGFR/AMIGO ligand polypeptides engage in a second interaction different from the first interaction. In a particular embodiment, the modulator is dominant negative form of the AMIGO, EGFR or AMIGO ligand polypeptide.

In one embodiment, the present invention provides AMIGO compounds that bind to epidermal growth factor receptor (EGFR), as well as compositions containing one or a combination of such compounds. The AMIGO compounds preferably inhibit (e.g., block) binding of EGFR ligands, such as EGF and TGF-.alpha., to EGFR or even more preferably inhibit the phosphorylation of EGFR. For example, binding of EGFR ligand to EGFR and/or EGFR phosphorylation can be inhibited by at least about 10%, 20%, 30%, 40%, 50%, 60%, 70%, 80%, 90%, or 100% and preferably results in the prevention of EGFR-mediated cell signaling.

In one embodiment exemplified herein, AMIGO compounds of the invention are AMIGO DNA constructs having an AMIGO cDNA cloned into a vector. Other AMIGO compounds are also encompassed by the invention, including AMIGO peptides, variants, biologically active fragments, an antigenic fragment of AMIGO, anti-AMIGO antibodies or binding portion thereof and and nucleic acids encoding said polypeptides that have retained their binding and/or EGFR phosphorylation inhibiting characteristics. The antibodies can be whole antibodies or antigen-binding fragments of the antibodies, including Fab, F(ab′).sub.2, Fv and chain Fv fragments.

### BRIEF DESCRIPTION OF THE DRAWINGS

FIGS. 1A and 1B. Cloning of AMIGO as an amphoterin-induced gene in hippocampal neurons. (1A) Analysis of ordered differential display on gel electrophoresis, from which the band corresponding to AMIGO was cut for sequencing (marked with arrow). Lane 1, sample from amphoterin matrix; lane 2, sample from laminin matrix. (1B) AMIGO induction was confirmed by using RT-PCR. Lane 1 contains an RT-PCR reaction from hippocampal neurons on amphoterin and lane 2 on laminin. Glyceraldehyde 3-phosphate dehydrogenase (GAPDH) was analysed as a control.

FIGS. 2A and 2B. Primary structure of human AMIGO, AMIGO2 and AMIGO3. (2A) Alingment of the three AMIGOs (SEQ ID NOS: 2, 4, and 6) where the identical amino acids between the all AMIGOs are highlighted in red with white letters and similar amino acids are highlighted in red with black letters. Different domains found in the AMIGOs are marked with coloured boxes above the sequences. (2B) Schematic view of the three AMIGOs.

FIG. 3. RT-PCR mRNA analysis of AMIGO, AMIGO2 and AMIGO3 in different adult mouse tissues. GAPDH, glyceraldehyde 3-phosphate dehydrogenase.

FIGS. 4A, 4B and 4C. In situ hybridization analysis of AMIGO mRNA. In E13 mouse embryo the AMIGO mRNA expression is highest in the dorsal root ganglia (DRG in 4A and 4B) and in the trigeminal ganglion (TG in 4A). (4C) In the adult mouse cerebrum the AMIGO expression is highest in the hippocampal formation where the most intense signal is seen in the dentate gyrus (DG). The pyramidal cell layers CA1-CA3 also express AMIGO.

FIGS. 5A and 5B. Characterization of the recombinant AMIGO Ig-fusion protein and of anti-AMIGO antibodies. (5A) The recombinant AMIGO Ig-fusion protein (lanes 1, 3 and 5) and protein lysates from adult brain (lanes 2, 4 and 6) were silver stained (lanes 1 and 2) or immunoblotted with rabbit anti-AMIGO antibodies (lanes 3-6). Anti-AMIGO identifies both the AMIGO Ig-fusion protein (lane 3) and one 65 kD band in rat brain lysate (lane 4). Binding of the antibodies to the band corresponding to AMIGO was inhibited by the peptide used for immunization (5A, lanes 5 and 6). Tissue sections were also inhibited in a dose-dependent manner by the peptide (shown for the immunohistochemistry of the adult cerebellum in 5B).

FIG. 6. AMIGO expression displays a dual character during brain development. Western blotting of AMIGO using crude brain extracts from different developmental stages reveals clear AMIGO expression during the late embryonic (E) and perinatal development, starting at the E14 stage. The AMIGO expression is downregulated during postnatal (P) stages P6-P10. The expression is again upregulated between the stages P10 and P12 and remains high in the adult brain. The upregulation coincides with the onset of myelination as demonstrated by the comparison with the CNPase expression. The expression of AMIGO and CNPase display a parallel increase during postnatal development. W; postnatal week.

FIGS. 7A, 7B, 7C, 7D, 7E and 7F. AMIGO is localized to axonal fiber tracts in tissue and in cultured cells. Immunohistochemical staining of rat tissues revealed that AMIGO is specifically expressed in the nervous system. In E15 embryo (7A) immunostaining is observed in developing fiber tracts and nerves, like in the ventral part of the marginal layer (ml) of the spinal cord (SC) and in the nerves connecting to the dorsal root ganglion (DRG) and to the spinal cord. In the adult animal (7B, 7C, 7D and 7E) AMIGO is also detected in nerve fibers. In the cerebellum (7B) the most intense staining is detected in fibers on both sides of the granule cell layer (G), as in the characteristic basket-like structure (arrow) formed by the basket cell axons around the Purkinje cell soma (p). Fibers in the cerebellar white matter (W) are also stained. In general, myelinated fiber tracts are clearly stained in adult animals as demonstrated by the similar staining of AMIGO (panel 7C) and CNPase (panel 7D) around the hippocampus in sagittal sections. In addition to the cerebellar basket cell axons, non-myelinated fibers are also stained in hippocampus (panel 7C, higher magnification in panel 7E). These CNPase negative fibers of the hippocampus reside in the vicinity of the CA3 pyramidal cell bodies. In cultured hippocampal neurons (panel 7F) AMIGO is also detected in neuronal processes by immunofluorescence staining. G, granule cell layer of the cerebellar cortex; M; molecular layer of the cerebellar cortex; CA1, CA1 region; CA3, CA3 region; h, hilus. Scale bar 50 μm in panels 7A, 7B, 7E and 7F; 500 μm in panels 7C and 7D.

FIGS. 8A, 8B, 8C and 8D. AMIGO promotes neurite outgrowth of hippocampal neurons.

(8A) Substratum coated with 25 μg/ml of AMIGO promotes neurite outgrowth of E18 hippocampal neurons. (8B) Cells on the control substratum coated with 25 μg/ml of the Fc protein without the AMIGO ectodomain is shown for comparison. (8C) AMIGO induced hippocampal neurite outgrowth after 24 h of culture. The AMIGO Ig-fusion (gray bars) and the Fc control protein (black bars) were coated as follows; 0 μg/ml (1), 3.125 μg/ml (2), 6.25 μg/ml (3), 12.5 μg/ml (4), 25 μg/ml (5), 50 μg/ml (6), 100 μg/ml (7). (8D) AMIGO-induced neurite outgrowth (the substratum coated with 25 μg/ml of the AMIGO Ig-fusion protein) is blocked by the AMIGO Ig-fusion protein in the assay medium. The AMIGO Ig-fusion (gray bars) and the Fc control protein (black bars) were added into the culture medium as follows; 0 μg/ml (1), 3.125 μg/ml (2), 6.25 μg/ml (3), 12.5 μg/ml (4), 25 μg/ml (5), 50 μg/ml (6), 100 μg/ml (7). The error bars give the standard deviation calculated from 15 microscopy fields in three independent experiments. Scale bar 50 μm in panels 8A and 8B.

FIGS. 9A, 9B, and 9C. Soluble AMIGO inhibits fasciculation in hippocampal neurons. (9A) E18 hippocampal neurons on poly-L-lysine substratum with 25 μg/ml of the AMIGO Ig-fusion in the culture medium. (9B) E18 hippocampal neurons on poly-L-lysine substratum with 25 μg/ml the Fc control protein is shown for comparison. (9C) Total length of processes, the diameter of which is <2 μm (formed from 1-3 neurites) on poly-L-lysine substratum. The AMIGO Ig-fusion and (gray bars) and the Fc control protein (black bars) were added into the culture medium as follows; 0 μg/ml (white bar)(1), 3.125 μg/ml (2), 6.25 μg/ml (3), 12.5 μg/ml (4), 25 μg/ml (5). The error bars in panel 9C give the standard deviation calculated from 12 microscopy fields in three independent experiments. Scale bar 50 μm in panel 9A and 9B.

FIGS. 10A, 10B, 10C, and 10D. Homophilic interaction of AMIGO. (10A) Co-immunoprecipitation experiment. Lane 1, cells transfected with full-length GFP-tagged AMIGO and full-length V5-tagged AMIGO; lane 2, full length GFP-tagged AMIGO and soluble V5-tagged AMIGO; lane 3, transfected only with full-length GFP-tagged AMIGO; lane 4, full length GFP-tagged AMIGO and full-length V5-tagged human RAGE. Full length GFP-tagged AMIGO was co-immunoprecipitated with full length V5-tagged AMIGO (lane 1) and by using soluble V5-tagged AMIGO containing only the ectodomain (lane 2) Co-immunoprecipitation could be also shown by precipitating with GFP antibody. (10B) Kinetics of bead aggregation. Nₜ and N₀ are the total number of particles at incubation times t and 0 respectively. The extent of bead aggregation is represented by the index Nₜ/N₀. Gray bars represent AMIGO Ig-fusion coated beads and black bars Fc coated beads. (10C-10D) Bead aggregation after 60 min using protein A beads coated with the AMIGO Ig-fusion (10C) or with the Fc control (10D). The error bars give the standard deviation calculated from 12 microscopy fields in three independent experiments.

FIG. 11. Multiple alignment for the leucine-rich repeat areas of Slit1, Nogo-receptor and AMIGO (SEQ ID NOS: 33-39). The identical amino acids between Slit1 and Nogo-receptor compared to the AMIGO are highlighted in black and similar amino acids are highlighted in gray. The consensus sequence of the 6 LRR motifs of the AMIGO are shown above the sequences.

FIG. 12. The three dimensional structure of the immunoglobulin domain, schematic presentation. Ig-domain is a sandwich of two antiparallel beta sheets. (Principles of Biochemistry, Horton et al. 2002)

FIGS. 13A and 13B. Structure of ribonuclease inhibitor. 13A) Ribbon diagram of the structure of porcine RI generated using the program MOLSCRIPT. 13B) Consensus sequences (SEQ ID NOS: 40-41) and secondary structure of LRRs of porcine RI. The sequence of RI was aligned so that two types of repeats (A and B) alternate in the sequence. One-letter amino acid code is used. ‘x’ indicates any amino acid and ‘a’ denotes an aliphatic amino acid. The part of the repeat that is strongly conserved in all LRR proteins is underlined, and the conserved residues are shown in bold. Below the sequence, solid lines mark the core region of Ig-sheet and helix; dots denote extensions of helix in different repeats. (Kobe and Deisenhofer 1995)

FIG. 14. Structure of RNase A-RI complex. In the ribbon diagram, RNase A is dark and RI is light. (Kobe and Deisenhofer 1995)

FIG. 15. Schematic drawings of the structures of some LRR-containing proteins. Tartan is a protein involved in Drosophila development (Chang et al. 1993, Milan et al. 2001). Slit protein contains additional domains, which are not represented here. Sig, signal peptide; AFR, amino terminal flanking region; LRR, leucine-rich repeat; CFR, carboxy terminal flanking region; PI, phosphatidylinositol. (Hayata et al. 1998)

FIG. 16. Schematic presentation of the predicted structure of AMIGO.

FIG. 17. Specificity of AMIGO staining in tissue. α-AMI (anti-AMIGO antibody) was incubated with rising concentrations of AMIP2 or AMIP1 peptide. Tissue sections from rat cerebellum were stained with peptide incubated antibody. Increasing concentration of AMIP2 peptide decreases and finally blocks the tissue staining completely. Evidently, the binding of α-AMI to tissue sections is inhibited by AMIP2 peptide. Control peptide AMIP1 does not have an effect on α-AMI binding even in high concentrations.

FIG. 18. Coronal section of the rat cerebrum stained with α-AMI. Myelinated fiber tracts are clearly stained. Some areas of the cerebral cortex are stained and one of them is marked with arrowhead (same areas are also stained with the oligodendrocyte marker α-CNPase and with α-NF-M). Non-myelinated structures are stained in the CA3 region of the hippocampus (arrow).

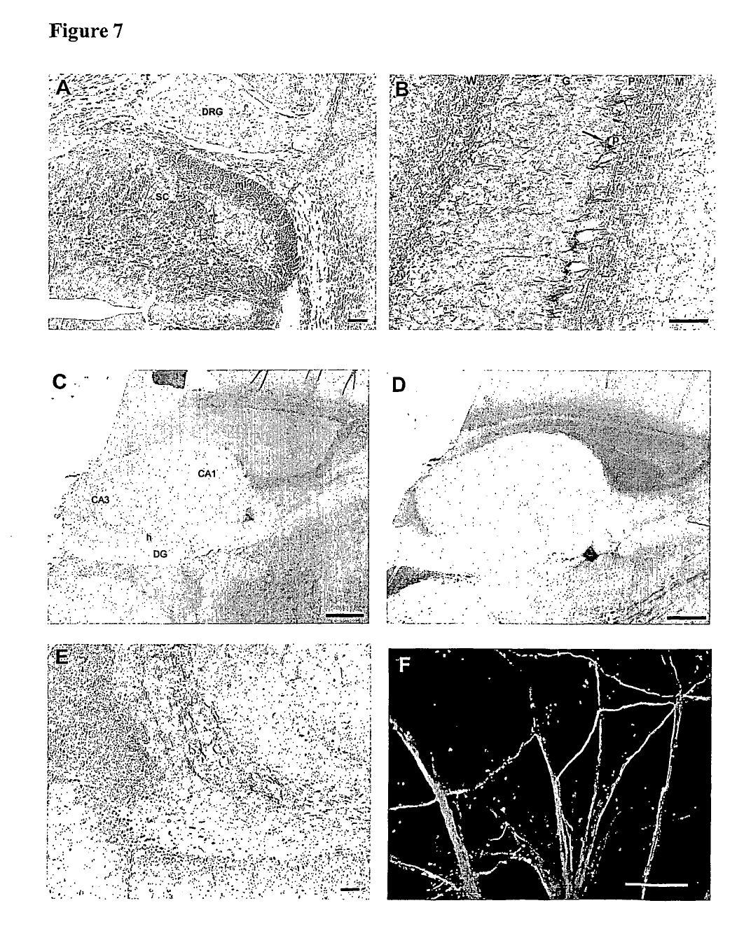

FIGS. 19A and 19B. Coronal section of the hippocampal CA3 region (arrow in FIG. 7, higher magnification). 19A) staining with α-AMI 19B) staining with α-NF-M. Both stainings are located near the proximal part of the apical dendrites of the pyramidal cells. This layer is called stratum lucidum. Less intensively stained round structures are the cell nuclei stained with hematoxylin. Scale bar 25 μm.

FIGS. 20A, 20B, 20C, and 20D. Sagittal sections of the rat hippocampus. 20a) staining with α-AMI 20b) staining with α-CNPase. Both antibodies stain the myelinated nerve fibers in and around the hippocampus. In addition, α-AMI causes strong staining of non-myelinated structures in the CA3 region of the hippocampus and in the hilus (h) of the dentate gyrus (DG). 20c) and 20d) Higher magnification of staining with α-AMI. The staining of the CA3 region and of the dentate gyrus has a fiber-like structure (arrows). Scale bar 50 μm.

FIGS. 21A and 21B. Higher magnification of the cerebral cortex. 21a) staining with α-AMI 21b) staining with α-NF-M. Same areas of the cerebral cortex are stained both with α-AMI and with α-NF-M. In closer examination, some of the thick apical dendrites of the pyramidal cells (arrows) and some thin fibers (arrowheads) are stained with both antibodies. Cell soma and basal dendrites of pyramidal cells are also stained with α-NF-M. Scale bar 50 μm.

FIGS. 22A and 22B. Cerebellar sections stained with α-AMI. 22a) Coronal section of the cerebellum. Cerebellar cortex consists of three layers: outermost molecular layer (M), Purkinje cell layer (P) and granule cell layer (G). White matter (W) underlies the cerebellar cortex. α-AMI appears to stain myelinated fibers in the white matter and in the granule cell layer. The basket-like structure (arrow) formed by the basket cell axons around the Purkinje cell soma (p) and fibers in the molecular layer are also stained. In the molecular layer the staining is restricted to the inner part of the layer and stained fibers mainly run parallel to Purkinje cell layer. 22b) Sagittal section of the cerebellum. In the medial part of the cerbellum, the structure of the staining in white matter (W) resembles a string of pearls. Scale bar 25 μm.

FIGS. 23A, 23B, and 23C. Transverse section of the spinal cord white matter. 23a) staining with α-AMI 23b) staining with α-CNPase 23c) staining with α-NF-M. Clear, round areas of the section are the myelin sheaths. Small spots (arrow) are clearly stained inside the myelin sheath with α-AMI and with α-NF-M. These spots seem to be the transections of axons. They are not stained with α-CNPase. The axon tracts running parallel to the section plane are stained with α-NF-M but not with α-AMI. Scale bar 50 μm.

FIGS. 24A and 24B. Immunohistochemistry of the kidney. Staining with α-AMI (24a) or with α-NF-M (24b) detects the same small structures in the kidney (arrows). Consequently, α-AMI staining is located in the nerves of the kidney. Scale bar 100 μm.

FIGS. 25A, 25B, 25C, and 25D. Immunohistochemistry of the head of 18 day old embryo. Staining with α-AMI detects developing fiber tracts and cranial nerves, like the optic nerve (in panel 25a) and the internal capsule (in panel 25c). Staining in retina (arrow in panel 25a) is located in the nerve fiber layer. Nerve fiber layer consist of ganglion cell axons, which form the optic nerve. Control sections (panel 25b and 25d) are stained with AMIP2 blocked α-AMI. Scale bar 100 μm.

FIGS. 26A and 26B. Western blotting of AMIGO using crude rat brain extracts from different developmental stages. Same total weight of tissue was used from each sample. Brains of 16- and 18-day old embryo (E16 and E18), of 1-, 2-, 4-, 6-, 8-, 10- and 14-day old rat (P1-P14) and of adult rat were used. AMIGOIg fusion protein (AMIIg) was used as a control sample. In panel 26a) Western blot is detected with α-AMI and α-CNPase. α-AMI detects about 65 kDa protein band and weaker protein band, about 130 kDa. α-CNPase detects about 48 kDa protein band. The AMIGO expression displays dual character during brain development. Immunoblotting reveals clear AMIGO expression during the late embryonic (E) and perinatal development. The AMIGO expression is downregulated during postnatal (P) stages P4-P10. The expression is again upregulated between stages P10 and P14 and remains high in the adult brain. The upregulation coincides with the onset of myelination as demonstrated by the comparison with the CNPase expression. The expression of AMIGO and CNPase display a parallel increase during postnatal development. In panel 26b) Western blot is stained with Ponceau stain to compare the total amount of protein in each sample.

FIG. 27. Coimmunoprecipitation of AMIGOs with EGFR. Stable EGFR expressing 293 cells were transfected with V5-tagged full length AMIGO (lane1), EC-part containing AMIGO (lane 2), full length AMIGO2 (lane 3) or with full length AMIGO3 (lane 4). The coimmunoprecipitations were done by using anti-EGFR antibodies and the detection was done by using anti-V5 antibodies. The result shows that both AMIGO and AMIGO2 bind the EGFR and only the EC-part is enough for the binding (shown for the AMIGO).

FIG. 28. Homo- and heterophilic binding of AMIGO, AMIGO2 and AMIGO3. Coimmunoprecipitation was done by using anti-V5-tag antibodies and the detection was done by using anti-GFP-tag antibodies. Lanes 1-5 contains full length AMIGO with GFP-tag; lanes 6-9 contains full length AMIGO2 with GFP-tag; lanes 10-12 full length AMIGO3 with GFP-tag. V5-tagged proteins used in this experiment: AMIGO full length in lane 1; AMIGO EC-part in lane 2; AMIGO2 full length in lanes 3 and 6; AMIGO2 EC-part in lane 7; AMIGO3 full length in lanes 4, 8 and 10; AMIGO3 EC-part in lane 11; RAGE full length in lanes 5, 9 and 12. Pictures shows that full length AMIGO-GFP could be co-immunoprecipitated with full length AMIGO, AMIGO2 and AMIGO3 but not with full length RAGE. Full length AMIGO-GFP could also be coimmunoprecipitated with only EC-part containing AMIGO. The full length AMIGO2-GFP could be coimmunoprecipitated with full length AMIGO2 and AMIGO3 but also with only EC-part containing AMIGO2. The full length AMIGO3-GFP could be coimmunoprecipitated with full length AMIGO3 and only EC-part containing AMIGO3. The coimmunoprecipitation results show that AMIGOs could bind each others in heterophilically but they also posses homophilic binding properties.

FIG. 29. AMIGO inhibits EGFR phosphorylation. When AMIGO and flag-tagged human EGFR are expressed together in HEK293T cells AMIGO clearly inhibits the EGFR autophosphorylation induced by EGF ligation when compared to AMIGO2, AMIGO3 and vector control.

### DETAILED DESCRIPTION OF THE INVENTION

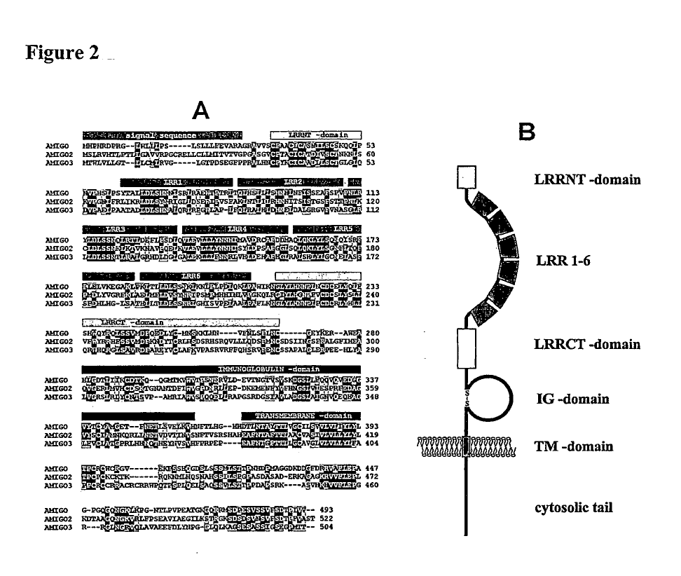

Amphoterin and laminin are both neurite outgrowth-promoting factors. The genes induced on amphoterin matrix were detected by using the ordered differential display method (Matz et al., 1997) from hippocampal neurons, which were cultured on amphoterin or laminin matrix. A novel gene was seen to be induced on amphoterin. The whole coding sequence for this differentially expressed gene was cloned and named as AMIGO (AMphoterin Induced Gene and Orphan receptor). The predicted amino acid sequences of the AMIGO codes type I transmembrane protein containing a signal sequence for secretion and a transmembrane domain. The deduced extracellular part of the AMIGO contains six leucine-rich repeats (LRRs) flanked by cysteine-rich LRRN- and LRRC-terminal domains and one immunoglobulin domain close to the transmembrane region. The deduced 100 amino acid long cytosolic part of the protein do not contain any known domains. We have also identified a novel family of transmembrane proteins consisting of AMIGO, AMIGO2 and AMIGO3. These three proteins show clear homology with each other; their length and location of different domains are highly identical (FIG. 2 B).

Based on RT-PCR experiments, in situ hybridization and immunohistochemistry, AMIGO is an essentially nervous system specific protein. One cellular mechanism in the growth of axonal connections is fasciculation: axons grow along each other by using pioneer axons as the substratum for the growth cones of the later ones. Interestingly, a dominant negative approach using AMIGO ectodomain in the culture medium clearly suggests a role for AMIGO in fasciculation. Further, AMIGO displays a homophilic binding mechanism that would explain its role in fasciculation. It is also noteworthy that the LRR sequences of the AMIGOs display homology with the slit proteins and with the Nogo receptor (FIG. 11) that have been implicated in axon growth, regeneration and guidance. The second upregulation of the AMIGO expression suggests a role in myelination. It seems reasonable that AMIGO would mediate cell-to-cell interactions also at this stage of development. Further, AMIGO expression remains high until adulthood. This suggests that AMIGO plays a role in regeneration and plasticity of the adult fiber tracts, the mechanisms of which commonly recapitulate mechanisms of fiber tract development.

Thus, this invention is based on the discovery and characterization of a novel human gene/protein termed AMIGO and its homologous counterparts designated as AMIGO2 and AMIGO3. Together these three proteins form a novel family of transmembrane proteins (for simplicity, all of these proteins are hereinafter referred as AMIGO or AMIGOs).

In one embodiment, the invention provides a purified protein comprising, or alternatively consisting of a polypeptide, a biologically active fragment, or an antigenic fragment of AMIGO.

In another embodiment the present invention is directed to proteins which comprise, or alternatively consist of, an amino acid sequence which is at least 80%, 85%, 90%, 95%, 96%, 97%, 98%, 99% or 100%, identical to AMIGO protein.

Due to the degeneracy of the genetic code, one of ordinary skill in the art will immediately recognize that a large number of the nucleic acid molecules having a sequence at least 80%, 85%, 90%, 95%, 96%, 97%, 98%, 99%, or 100% identical to the nucleic acid sequence of the cDNA contained in SEQ ID NO:1, 3, or 5 or fragments thereof, will encode polypeptides “having functional activity.” In fact, since degenerate variants of any of these nucleotide sequences all encode the same polypeptide, in many instances, this will be clear to the skilled artisan. It will be further recognized in the art that, for such nucleic acid molecules that are not degenerate variants, a reasonable number will also encode a polypeptide having functional activity. This is because the skilled artisan is fully aware of amino acid substitutions that are either less likely or not likely to significantly effect protein function (e.g. replacing one aliphatic amino acid with a second aliphatic amino acid), as further described below.

For example, guidance concerning how to make phenotypically silent amino acid substitutions is provided in Bowie et al., “Deciphering the Message in Protein Sequences: Tolerance to Amino Acid Substitutions,” Science 247:1306-1310 (1990), wherein the authors indicate that there are two main strategies for studying the tolerance of an amino acid sequence to be changed.

The first strategy exploits the tolerance of amino acid substitutions by natural selection during the process of evolution. By comparing amino acid sequences in different species, conserved amino acids can be identified. These conserved amino acids are likely important for protein function. In contrast, the amino acid positions where substitutions have been tolerated by natural selection indicates that these positions are not critical for protein function. Thus, positions tolerating amino acid substitution could be modified while still maintaining biological activity of the protein.

In addition to naturally occurring allelic variants of AMIGO, changes can be introduced by mutation into AMIGO sequences that incur alterations in the amino acid sequences of the encoded AMIGO polypeptide. Nucleotide substitutions leading to amino acid substitutions at “non-essential” amino acid residues can be made in the sequence of an AMIGO polypeptide. A “non-essential” amino acid residue is a residue that can be altered from the wild-type sequences of AMIGO without altering its biological activity, whereas an “essential” amino acid residue is required for such biological activity. For example, amino acid residues that are conserved among the AMIGO molecules of the invention are predicted to be particularly non-amenable to alteration. Amino acids for which conservative substitutions can be made are well known in the art. Useful conservative substitutions are shown in Table B, “Preferred substitutions.” Conservative substitutions whereby an amino acid of one class is replaced with another amino acid of the same type fall within the scope of the subject invention so long as the substitution does not materially alter the biological activity of the compound.

The second strategy uses genetic engineering to introduce amino acid changes at specific positions of a cloned gene to identify regions critical for protein function. For example, site directed mutagenesis or alanine-scanning mutagenesis (introduction of single alanine mutations at every residue in the molecule) could be used. See Cunningham et al., Science 244:1081-1085 (1989). The resulting mutant molecules can then be tested for biological activity. Besides conservative amino acid substitutions (See Table B below), variants of the present invention include (i) substitutions with one or more of the non-conserved amino acid residues, where the substituted amino acid residues may or may not be one encoded by the genetic code, or (ii) substitutions with one or more of the amino acid residues having a substituent group, or (iii) fusion of the mature polypeptide with another compound, such as a compound to increase the stability and/or solubility of the polypeptide (for example, 8961 polyethylene glycol), or (iv) fusion of the polypeptide with additional amino acids, such as, for example, an IgG Fc fusion peptide, serum albumin (preferably human serum albumin) or a fragment or variant thereof, or leader or secretory sequence, or a sequence facilitating purification. Such variant polypeptides are deemed to be within the scope of those skilled in the art from the teachings herein.

TABLE B
Preferred substitutions

| Original | Exemplary | Preferred |
|---|---|---|
| residue | substitutions | substitutions |
| Ala (A) | Val, Leu, Ile | Val |
| Arg (R) | Lys, Gln, Asn | Lys |
| Asn (N) | Gln, His, Lys, Arg | Gln |
| Asp (D) | Glu | Glu |
| Cys (C) | Ser | Ser |
| Gln (Q) | Asn | Asn |
| Glu (E) | Asp | Asp |
| Gly (G) | Pro, Ala | Ala |
| His (H) | Asn, Gln, Lys, Arg | Arg |
| Ile (I) | Leu, Val, Met, Ala, | Leu |
| | Phe, Norleucine | |
| Leu (L) | Norleucine, Ile, | Ile |
| | Val, Met, Ala, Phe | |
| Lys (K) | Arg, Gln, Asn | Arg |
| Met (M) | Leu, Phe, Ile | Leu |
| Phe (F) | Leu, Val, Ile, Ala, | Leu |
| | Tyr | |
| Pro (P) | Ala | Ala |
| Ser (S) | Thr | Thr |
| Thr (T) | Ser | Ser |
| Trp (W) | Tyr, Phe | Tyr |
| Tyr (Y) | Trp, Phe, Thr, Ser | Phe |
| Val (V) | Ile, Leu, Met, Phe, | Leu |
| | Ala, Norleucine | |

A further embodiment of the invention relates to polypeptides which comprise the amino acid sequence of a polypeptide having an amino acid sequence which contains at least one amino acid substitution, but not more than 50 amino acid substitutions, even more preferably, not more than 40 amino acid substitutions, still more preferably, not more than 30 amino acid substitutions, and still even more preferably, not more than 20 amino acid substitutions from a polypeptide sequence disclosed herein. It is highly preferable for a polypeptide to have an amino acid sequence which comprises the amino acid sequence of a polypeptide, a portion, or a complement of SEQ ID NO:2, 4 or 6 in order of ever-increasing preference, at least one, but not more than 10, 9, 8, 7, 6, 5, 4, 3, 2 or 1 amino acid substitutions.

In preferred embodiments, the amino acid substitutions are conservative.

In specific embodiments, the polypeptides of the invention comprise, or alternatively, consist of, fragments or variants of a reference amino acid sequence encoded by SEQ ID NO:2, 4 or 6 wherein the fragments or variants have 1-5, 5-10, 5-25, 5-50, 10-50 or 50-150, amino acid residue additions, substitutions, and/or deletions when compared to the reference amino acid sequence.

In one embodiment techniques suitable for the production of AMIGO polypeptide are well known in the art and include isolating AMIGO from an endogenous source of the polypeptide, peptide synthesis (using a peptide synthesizer) and recombinant techniques (or any combination of these techniques).

In one embodiment, the isolated nucleic acid molecule comprises a nucleotide sequence encoding a protein, wherein the protein comprises an amino acid sequence at least about 45%, preferably 60%, more preferably 70%, 80%, 90%, and most preferably about 95% homologous to that of an AMIGO.

In another embodiment, AMIGO polypeptide variants have at least (I) about 80% amino acid sequence identity with a full-length native AMIGO polypeptide sequence shown in SEQ ID NO:2, 4 or 6 (2) an AMIGO polypeptide sequence lacking the signal peptide, (3) any other fragment of a full-length AMIGO polypeptide sequence. For example, AMIGO polypeptide variants include AMIGO polypeptides wherein one or more amino acid residues are added or deleted at the N- or C-terminus of the full-length native amino acid sequence. An AMIGO polypeptide variant will have at least about 80% amino acid sequence identity, preferably at least about 81% amino acid sequence identity, more preferably at least about 82%, 83%, 84%, 85%, 86%, 87%, 88%, 89%, 90%, 91%, 92%, 93%, 94%, 95%, 96%, 97%, 98% amino acid sequence identity and most preferably at least about 99% amino acid sequence identity with a full-length native sequence AMIGO polypeptide sequence. An AMIGO polypeptide variant may have a sequence lacking the signal peptide or any other fragment of a full-length AMIGO polypeptide sequence. Ordinarily, AMIGO variant polypeptides are at least about 10 amino acids in length, often at least about 20 amino acids in length, more often at least about 30, 40, 50, 60, 70, 80, 90, 100 or 150 amino acids in length, or more.

One aspect of the invention provides an isolated nucleic acid molecule comprising, or alternatively consisting of, a polynucleotide having a nucleotide sequence selected from the group consisting of (a) a nucleotide sequence described in SEQ ID NO:1, 3 or 5 (b) a nucleotide sequence in SEQ ID NO:1, 3 or 5 part of which encodes a mature AMIGO polypeptide; (c) a nucleotide sequence which encodes a biologically active fragment of an AMIGO polypeptide; (d) a nucleotide sequence which encodes an antigenic fragment of an AMIGO polypeptide; (e) a nucleotide sequence complementary to any of the nucleotide sequences in (a), (b), (c), (d), above.

The invention further encompasses nucleic acid molecules that differ from the nucleotide sequences due to degeneracy of the genetic code and thus encode the same AMIGO protein as shown in sequence of SEQ ID NO:2, 4 or 6.

In addition sequence polymorphisms that change the amino acid sequences of the AMIGO may exist within a population. For example, allelic variation among individuals will exhibit genetic polymorphism in AMIGO. The terms “gene” and “recombinant gene” refer to nucleic acid molecules comprising an open reading frame (ORF) encoding AMIGO, preferably a vertebrate AMIGO. Such natural allelic variations can typically result in 1-5% variance in AMIGO. Any and all such nucleotide variations and resulting amino acid polymorphisms in the AMIGO, which are the result of natural allelic variation and that do not alter the functional activity of the AMIGO are within the scope of the invention.

Moreover, AMIGO from other species that have a nucleotide sequence that differs from the human sequence of AMIGO are contemplated. Nucleic acid molecules corresponding to natural allelic variants and homologues of AMIGO cDNAs of the invention can be isolated based on their homology to AMIGO using cDNA-derived probes to hybridize to homologous AMIGO sequences under stringent conditions.

“AMIGO variant polynucleotide” or “AMIGO variant nucleic acid sequence” means a nucleic acid molecule which encodes an active AMIGO that (1) has at least about 80% nucleic acid sequence identity with a nucleotide acid sequence encoding a full-length native AMIGO, (2) a full-length native AMIGO lacking the signal peptide, or (3) any other fragment of a full-length AMIGO. Ordinarily, an AMIGO variant polynucleotide will have at least about 80% nucleic acid sequence identity, more preferably at least about 81%, 82%, 83%, 84%, 85%, 86%, 87%, 88%, 89%, 90%, 91%, 92%, 93%, 94%, 95%, 96%, 97%, 98% nucleic acid sequence identity and yet more preferably at least about 99% nucleic acid sequence identity with the nucleic acid sequence encoding a full-length native AMIGO. An AMIGO variant polynucleotide may encode full-length native AMIGO lacking the signal peptide with or without the signal sequence, or any other fragment of a full-length AMIGO. Variants do not encompass the native nucleotide sequence.

Ordinarily, AMIGO variant polynucleotides are at least about 30 nucleotides in length, often at least about 60, 90, 120, 150, 180, 210, 240, 270, 300, 400 nucleotides in length, more often at least about 500 nucleotides in length, or more.

The structure and sequence of the mammalian AMIGO cDNA sequence which encodes the mouse and human sequences disclosed herein, make it possible to clone gene sequences from other mammals which encode the AMIGO. Of particular interest to the present invention is the ability to clone the human AMIGO molecules using the sequences disclosed herein. The DNA encoding AMIGO may be obtained from any cDNA library prepared from tissue believed to possess the AMIGO mRNA and to express it at a detectable level, as shown herein in the Examples. Accordingly, AMIGO DNA can be conveniently obtained from a cDNA library prepared, for example, from mammalian fetal liver, brain, muscle, intestine, and peripheral nerves. The AMIGO-encoding gene may also be obtained from a genomic library or by oligonucleotide synthesis.

Libraries are screened with probes (such as antibodies to the AMIGO or oligonucleotides of about 20-80 bases) designed to identify the gene of interest or the protein encoded by it. Screening the cDNA or genomic library with the selected probe may be conducted using standard procedures as described in chapters 10-12 of Sambrook et al., Molecular Cloning: A Laboratory Manual (New York: Cold Spring Harbor Laboratory Press, 1989) or alternatively to use PCR methodology as described in section 14 of Sambrook et al., supra.

Amino acid sequence variants of AMIGO are prepared by introducing appropriate nucleotide changes into the AMIGO DNA, or by synthesis of the desired AMIGO polypeptide. Such variants represent insertions, substitutions, and/or specified deletions of, residues within or at one or both of the ends of the amino acid sequence of a naturally occurring AMIGO with sequence of SEQ ID NO:2, 4 or 6. Preferably, these variants represent insertions and/or substitutions within or at one or both ends of the mature sequence, and/or insertions, substitutions and/or specified deletions within or at one or both of the ends of the signal sequence of the AMIGO. Any combination of insertion, substitution, and/or specified deletion is made to arrive at the final construct, provided that the final construct possesses the desired biological activity as defined herein.

Variations in the native sequence as described above can be made using any of the techniques and guidelines for conservative and non-conservative mutations set forth in U.S. Pat. No. 5,364,934. These include oligonucleotide-mediated (site-directed) mutagenesis, alanine scanning, and PCR mutagenesis.

The nucleic acid (e.g., cDNA or genomic DNA) encoding the AMIGO is inserted into a replicable vector for further cloning (amplification of the DNA) or for expression. Many vectors are available. The vector components generally include, but are not limited to, one or more of the following: a signal sequence, an origin of replication, one or more marker genes, an enhancer element, a promoter, and a transcription termination sequence.

The AMIGOs of this invention may be produced recombinantly not only directly, but also as a fusion polypeptide with a heterologous polypeptide, which is preferably a signal sequence or other polypeptide having a specific cleavage site at the N-terminus of the mature protein or polypeptide. Fusion proteins can be easily created using recombinant methods. A nucleic acid encoding AMIGO can be fused in-frame with a non-AMIGO encoding nucleic acid, to the AMIGO N- or COOH-terminus, or internally. Fusion genes may also be synthesized by conventional techniques, including automated DNA synthesizers. An AMIGO fusion protein may include any portion to the entire AMIGO, including any number of the biologically active portions. Fusion polypeptides are useful in expression studies, cell-localization, bioassays, and AMIGO purification

Alternatively, AMIGO fusion protein can also be easily created using PCR amplification and anchor primers that give rise to complementary overhangs between two consecutive gene fragments that can subsequently be annealed and reamplified to generate a chimeric gene sequence (Ausubel et al., supra).

The signal sequence may be a component of the vector, or it may be a part of the AMIGO DNA that is inserted into the vector. The heterologous signal sequence selected preferably is one that is recognized and processed (i.e., cleaved by a signal peptidase) by the host cell. For prokaryotic host cells that do not recognize and process the native AMIGO signal sequence, the signal sequence is substituted by a prokaryotic signal sequence selected, for example, from the group of the alkaline phosphatase, penicillinase, or heat-stable enterotoxin II leaders. For yeast secretion the native signal sequence may be substituted by, e.g., the yeast invertase leader, alpha-factor leader (including Saccharomyces and Kluyveromyces, alpha-factor leaders, the latter described in U.S. Pat. No. 5,010,182 issued Apr. 23, 1991), or acid phosphatase leader, the Candida albicans glucoamylase leader (EP 362,179 published Apr. 4, 1990). In mammalian cell expression the native signal sequence (e.g., the AMIGO presequence that normally directs secretion of AMIGO from human cells in vivo) is satisfactory, although other mammalian signal sequences may be suitable, such as signal sequences from other animal AMIGOs, and signal sequences from secreted polypeptides of the same or related species, as well as viral secretory leaders, for example, the herpes simplex gD signal.

Expression and cloning vectors usually contain a promoter that is recognized by the host organism and is operably linked to the AMIGO nucleic acid. Vector choice is dictated by the organism or cells being used and the desired fate of the vector. Vectors may replicate once in the target cells, or may be “suicide” vectors. In general, vectors comprise signal sequences, origins of replication, marker genes, enhancer elements, promoters, and transcription termination sequences. The choice of these elements depends on the organisms in which the vector will be used and are easily determined. Some of these elements may be conditional, such as an inducible or conditional promoter that is turned “on” when conditions are appropriate.

Vectors can be divided into two general classes: Cloning vectors are replicating plasmid or phage with regions that are non-essential for propagation in an appropriate host cell, and into which foreign DNA can be inserted; the foreign DNA is replicated and propagated as if it were a component of the vector. An expression vector (such as a plasmid, yeast, or animal virus genome) is used to introduce foreign genetic material into a host cell or tissue in order to transcribe and translate the foreign DNA. In expression vectors, the introduced DNA is operably linked to elements, such as promoters, that signal to the host cell to transcribe the inserted DNA. Some promoters are exceptionally useful, such as inducible promoters that control gene transcription in response to specific factors. Operably linking AMIGO or anti-sense construct to an inducible promoter can control the expression of AMIGO or fragments, or anti-sense constructs. Examples of classic inducible promoters include those that are responsive to a-interferon, heat-shock, heavy metal ions, and steroids such as glucocorticoids (Kaufman R J, Vectors Used for Expression in Mammalian Cells,” Methods in Enzymology, Gene Expression Technology, David V. Goeddel, ed., 1990, 185:487-511) and tetracycline. Other desirable inducible promoters include those that are not endogenous to the cells in which the construct is being introduced, but, however, is responsive in those cells when the induction agent is exogenously supplied.

Promoters are untranslated sequences located upstream (5′) to the start codon of a structural gene (generally within about 100 to 1000 bp) that control the transcription and translation of particular nucleic acid sequence, such as the AMIGO nucleic acid sequence, to which they are operably linked. Such promoters typically fall into two classes, inducible and constitutive. Inducible promoters are promoters that initiate increased levels of transcription from DNA under their control in response to some change in culture conditions, e.g., the presence or absence of a nutrient or a change in temperature. At this time a large number of promoters recognized by a variety of potential host cells are well known. These promoters are operably linked to AMIGO-encoding DNA by removing the promoter from the source DNA by restriction enzyme digestion and inserting the isolated promoter sequence into the vector. Both the native AMIGO promoter sequence and many heterologous promoters may be used to direct amplification and/or expression of the AMIGO DNA. However, heterologous promoters are preferred, as they generally permit greater transcription and higher yields of AMIGO as compared to the native AMIGO promoter. Various promoters exist for use with prokaryotic, eukaryotic, yeast and mammalian host cells, known for skilled artisan.

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding AMIGO.

Construction of suitable vectors containing one or more of the above-listed components employs standard ligation techniques. Isolated plasmids or DNA fragments are cleaved, tailored, and religated in the form desired to generate the plasmids required.

Particularly useful in the practice of this invention are expression vectors that provide for the transient expression in mammalian cells of DNA encoding AMIGO. In general, transient expression involves the use of an expression vector that is able to replicate efficiently in a host cell, such that the host cell accumulates many copies of the expression vector and, in turn, synthesizes high levels of a desired polypeptide encoded by the expression vector, Sambrook et al., supra, pp. 16.17-16.22. Transient expression systems, comprising a suitable expression vector and a host cell, allow for the convenient positive identification of polypeptides encoded by cloned DNAs, as well as for the rapid screening of such polypeptides for desired biological or physiological properties. Thus, transient expression systems are particularly useful in the invention for purposes of identifying analogs and variants of AMIGO that are biologically active.

Propagation of vertebrate cells in culture (tissue culture) has become a routine procedure. See, e.g., Tissue Culture, Academic Press, Kruse and Patterson, editors (1973). Examples of useful mammalian host cell lines are monkey kidney CV1 line transformed by SV40 (COS-7, ATCC CRL 1651); Chinese hamster ovary cells/-DHFR (CHO, Urlaub et al., Proc. Natl. Acad. Sci USA, 77:4216 (1980)); human cervical carcinoma cells (HELA, ATCC CCL 2); and canine kidney cells (MDCK, ATCC CCL 34);

Host cells are transfected and preferably transformed with the above-described expression or cloning vectors for AMIGO production and cultured in conventional nutrient media modified as appropriate for inducing promoters, selecting transformants, or amplifying the genes encoding the desired sequences.

Transfection refers to the taking up of an expression vector by a host cell whether or not any coding sequences are in fact expressed. Numerous methods of transfection are known to the ordinarily skilled artisan, for example, electroporation. Successful transfection is generally recognized when any indication of the operation of this vector occurs within the host cell.

Transformation means introducing DNA into an organism so that the DNA is replicable, either as an extrachromosomal element or by chromosomal integrant. Depending on the host cell used, transformation is done using standard techniques appropriate to such cells. The calcium treatment employing calcium chloride, as described in section 1.82 of Sambrook et al., supra, or electroporation is generally used for prokaryotes or other cells that contain substantial cell-wall barriers.

General aspects of mammalian cell host system transformations have been described in U.S. Pat. No. 4,399,216 issued Aug. 16, 1983. Transformations into yeast are typically carried out according to the method of Van Solingen et al., J. Bact., 130:946 (1977) and Hsiao et al., Proc. Natl. Acad. Sci. USA, 76:3829 (1979). However, other methods for introducing DNA into cells, such as by nuclear microinjection, electroporation, bacterial protoplast fusion with intact cells, or polycations, e.g., polybrene, polyomithine, etc., may also be used. For various techniques for transforming mammalian cells, see Keown et al., Methods in Enzymology, 185:527-537 (1990) and Mansour et al., Nature, 336:348-352 (1988).

Prokaryotic cells used to produce the AMIGO polypeptide of this invention are cultured in suitable media as described generally in Sambrook et al., supra. In general, principles, protocols, and practical techniques for maximizing the productivity of mammalian cell cultures can be found in Mammalian Cell Biotechnology: a Practical Approach, M. Butler, ed. (IRL Press, 1991).

Gene amplification and/or expression may be measured in a sample directly, for example, by conventional Southern blotting, Northern blotting to quantitate the transcription of mRNA (Thomas, Proc. Natl. Acad. Sci. USA, 77:5201-5205 (1980)), dot blotting (DNA analysis), or in situ hybridization, using an appropriately labeled probe, based on the sequences provided herein. Various labels may be employed, most commonly radioisotopes, particularly ³²P. However, other techniques may also be employed, such as using biotin-modified nucleotides for introduction into a polynucleotide or antibodies recognizing specific duplexes, including DNA duplexes, RNA duplexes, and DNA-RNA hybrid duplexes or DNA-protein duplexes.

Gene expression, alternatively, can be measured by immunological methods, such as immunohistochemical staining of tissue sections and assay of cell culture or body fluids, to quantitate directly the expression of gene product. With immunohistochemical staining techniques, a cell sample is prepared, typically by dehydration and fixation, followed by reaction with labeled antibodies specific for the gene product coupled, where the labels are usually visually detectable, such as enzymatic labels, fluorescent labels, luminescent labels, and the like. A particularly sensitive staining technique suitable for use in the present invention is described by Hsu et al., Am. J. Clin. Path., 75:734-738 (1980).

Recombinant Production

When AMIGO is produced in a recombinant cell other than one of human origin, the AMIGO is completely free of proteins or polypeptides of human origin. However, it is necessary to purify AMIGO from recombinant cell proteins or polypeptides to obtain preparations that are substantially homogeneous as to AMIGO. As a first step, the culture medium or lysate can be centrifuged to remove particulate cell debris. AMIGO can then be purified from contaminant soluble proteins and polypeptides with the following procedures, which are exemplary of suitable purification procedures: by fractionation on an ion-exchange column; ethanol precipitation; reverse phase HPLC; chromatography on silica; chromatofocusing; immunoaffinity; epitope-tag binding resin; SDS-PAGE; ammonium sulfate precipitation; gel filtration using, for example, Sephadex G-75; and protein A Sepharose columns to remove contaminants such as IgG.

AMIGO variants in which residues have been deleted, inserted, or substituted are recovered in the same fashion as native sequence AMIGO, taking account of any substantial changes in properties occasioned by the variation. Immunoaffinity resins, such as a monoclonal anti-AMIGO resin, can be employed to absorb the AMIGO variant by binding it to at least one remaining epitope.

Variants can be assayed as taught herein. A change in the immunological character of the AMIGO molecule, such as affinity for a given antibody, can be measured by a competitive-type immunoassay. Other potential modifications of protein or polypeptide properties such as redox or thermal stability, hydrophobicity, susceptibility to proteolytic degradation, or the tendency to aggregate with carriers or into multimers are assayed by methods well known in the art.

This invention encompasses chimeric polypeptides comprising AMIGO fused to a heterologous polypeptide. A chimeric AMIGO is one type of AMIGO variant as defined herein. In one preferred embodiment, the chimeric polypeptide comprises a fusion of the AMIGO with a tag polypeptide which provides an epitope to which an anti-tag antibody or molecule can selectively bind. The epitope-tag is generally provided at the amino- or carboxyl-terminus of the AMIGO. Such epitope-tagged forms of the AMIGO are desirable, as the presence thereof can be detected using a labeled antibody against the tag polypeptide. Also, provision of the epitope tag enables the AMIGO to be readily purified by affinity purification using the anti-tag antibody. Affinity purification techniques and diagnostic assays involving antibodies are described later herein.

Tag polypeptides and their respective antibodies are well known in the art. Examples include the flu HA tag polypeptide and its antibody 12CA5 (Field et al., Mol. Cell. Biol., 8:2159-2165 (1988)); the c-myc tag and the 8F9, 3C7, 6E10, G4, B7 and 9E10 antibodies thereto (Evan et al., Molecular and Cellular Biology, 5:3610-3616 (1985)); and the Herpes Simplex virus glycoprotein D (gD) tag and its antibody (Paborsky et al., Protein Engineering, 3(6):547-553 (1990)). Other tag polypeptides have been disclosed. Examples include the Flag-peptide (Hopp et al., BioTechnology, 6:1204-1210 (1988)); the KT3 epitope peptide (Martin et al., Science, 255:192-194 (1992)); an alpha-tubulin epitope peptide (Skinner et al., J. Biol. Chem., 266:15163-15166 (1991)); and the T7 gene protein peptide tag (Lutz-Freyermuth et al., Proc. Natl. Acad. Sci. USA, 87:6393-6397 (1990)).

Once the tag polypeptide has been selected, an antibody thereto can be generated using the techniques disclosed herein. A C-terminal poly-histidine sequence tag is preferred. Poly-histidine sequences allow isolation of the tagged protein by Ni-NTA chromatography as described (Lindsay et al. Neuron 17:571-574 (1996)), for example.

The general methods suitable for the construction and production of epitope-tagged AMIGO are the same as those disclosed hereinabove.

Epitope-tagged AMIGO can be conveniently purified by affinity chromatography using the anti-tag antibody. The matrix to which the affinity antibody is attached is most often agarose, but other matrices are available (e.g. controlled pore glass or poly(styrenedivinyl)benzene). The epitope-tagged AMIGO can be eluted from the affinity column by varying the buffer pH or ionic strength or adding chaotropic agents, for example.

Chimeras constructed from an AMIGO sequence linked to an appropriate immunoglobulin constant domain sequence (immunoadhesins) are known in the art. Immunoadhesins reported in the literature include fusions of the T cell receptor (Gascoigne et al., Proc. Natl. Acad. Sci. USA, 84: 2936-2940 (1987)); CD4* (Capon et al., Nature 337: 525-531 (1989); Traunecker et al., Nature, 339: 68-70 (1989); Zettmeissl et al., DNA Cell Biol USA, 9: 347-353 (1990); Byrn et al., Nature, 344: 667-670 (1990)); TNF receptor (Ashkenazi et al., Proc. Natl. Acad. Sci. USA, 88: 10535-10539 (1991); Lesslauer et al., Eur. J. Immunol., 27: 2883-2886 (1991); Peppel et al., J. Exp. Med., 174:1483-1489 (1991)); and IgE receptor alpha* (Ridgway et al., J. Cell. Biol., 115:abstr. 1448 (1991)), where the asterisk (*) indicates that the receptor is member of the immunoglobulin superfamily.

The simplest and most straightforward immunoadhesin design combines the binding region(s) of the “adhesin” protein with the hinge and Fc regions of an immunoglobulin heavy chain. Ordinarily, when preparing the AMIGO-immunoglobulin chimeras of the present invention, nucleic acid encoding the AMIGO will be fused C-terminally to nucleic acid encoding the N-terminus of an immunoglobulin constant domain sequence, however N-terminal fusions are also possible.

Typically, in such fusions the encoded chimeric polypeptide will retain at least functionally active hinge and CH2 and CH3 domains of the constant region of an immunoglobulin heavy chain. Fusions are also made to the C-terminus of the Fc portion of a constant domain, or immediately N-terminal to the CH1 of the heavy chain or the corresponding region of the light chain.

The precise site at which the fusion is made is not critical; particular sites are well known and may be selected in order to optimise the biological activity, secretion or binding characteristics of the AMIGO-immunoglobulin chimeras.

The choice of host cell line for the expression of AMIGO immunoadhesins depends mainly on the expression vector. Another consideration is the amount of protein that is required. Milligram quantities often can be produced by transient transfections utilizing, for example, calcium phosphate or DEAE-dextran method (Aruffo et al., Cell, 61:1303-1313 (1990); Zettmeissl et al., DNA Cell Biol. US, 9:347-353 (1990)). If larger amounts of protein are desired, the immunoadhesin can be expressed after stable transfection of a host cell line, for example, introducing the expression vectors into Chinese hamster ovary (CHO) cells in the presence of an additional plasmid encoding dihydrofolate reductase.

Antibodies

AMIGO nucleic acid is useful for the preparation of AMIGO polypeptide by recombinant techniques exemplified herein which can then be used for production of anti-AMIGO antibodies having various utilities described below.

Antibodies useful for immunohistochemical staining and/or assay of sample fluids may be either monoclonal or polyclonal.

The invention further includes an antibody that specifically binds with AMIGO, or a fragment thereof. In a preferred embodiment, the invention includes an antibody that inhibits the biological activity of AMIGO. The antibody is useful for the identification for AMIGO in a diagnostic assay for the determination of the levels of AMIGO in a mammal having a disease associated with AMIGO levels. In addition, an antibody that specifically binds AMIGO is useful for blocking the interaction between AMIGO and its receptor, and is therefore useful in a therapeutic setting for treatment of AMIGO related disease, as described herein.

Monoclonal antibodies directed against full length or peptide fragments of an AMIGO protein or peptide may be prepared using any well-known monoclonal antibody preparation procedures, such as those described, for example, in Harlow et al. (1988, In: Antibodies, A Laboratory Manual, Cold Spring Harbor, N.Y.). Anti-AMIGO mAbs may be prepared using hybridoma methods comprising at least four steps: (1) immunizing a host, or lymphocytes from a host; (2) harvesting the mAb secreting (or potentially secreting) lymphocytes, (3) fusing the lymphocytes to immortalized cells, and (4) selecting those cells that secrete the desired (anti-AMIGO) mAb. The mAbs may be isolated or purified from the culture medium or ascites fluid by conventional Ig purification procedures such as protein A-Sepharose, hydroxylapatite chromatography, gel electrophoresis, dialysis, ammonium sulfate precipitation or affinity chromatography (Harlow et al, supra).

A mouse, rat, guinea pig, hamster, or other appropriate host is immunized to elicit lymphocytes that produce or are capable of producing Abs that will specifically bind to the immunogen. Alternatively, the lymphocytes may be immunized in vitro.

If human cells are desired, peripheral blood lymphocytes are generally used; however, spleen cells or lymphocytes from other mammalian sources are preferred.

The immunogen typically includes AMIGO or an AMIGO fusion protein.

The invention further comprises humanized and human anti-AMIGO Abs.

Humanized forms of non-human Abs are chimeric Igs, Ig chains or fragments (such as Fv, Fab, Fab′, F(ab′) or other antigen-binding subsequences of Abs) that contain minimal sequence derived from non-human Ig.

Generally, a humanized antibody has one or more amino acid residues introduced from a non-human source. These non-human amino acid residues are often referred to as “import” residues, which are typically taken from an “import” variable domain. Humanization is accomplished by substituting rodent CDRs or CDR sequences for the corresponding sequences of a human antibody (Jones et al., Nature 321:522-525 (1986); Riechmann et al., Nature 332:323-327 (1988); Verhoeyen et al., Science 239:1534-1536, (1988). Such “humanized” Abs are chimeric Abs (U.S. Pat. No. 4,816,567, 1989), wherein substantially less than an intact human variable domain has been substituted by the corresponding sequence from a non-human species. In practice, humanized Abs are typically human Abs in which some CDR residues and possibly some FR residues are substituted by residues from analogous sites in rodent Abs. Humanized Abs include human Igs (recipient antibody) in which residues from a complementary determining region (CDR) of the recipient are replaced by residues from a CDR of a non-human species (donor antibody) such as mouse, rat or rabbit, having the desired specificity, affinity and capacity. In some instances, corresponding non-human residues replace Fv framework residues of the human Ig. Humanized Abs may comprise residues that are found neither in the recipient antibody nor in the imported CDR or framework sequences. In general, the humanized antibody comprises substantially all of at least one, and typically two, variable domains, in which most if not all of the CDR regions correspond to those of a non-human Ig and most if not all of the FR regions are those of a human Ig consensus sequence. The humanized antibody optimally also comprises at least a portion of an Ig constant region typically that of a human Ig (Jones et al., supra; Presta L G, Curr Opin Biotechnol 3:394-398 (1992).

Human Abs can also be produced using various techniques, including phage display libraries (Hoogenboom et al., Nucleic Acids Res 19:4133-4137 (1991); Marks et al., Biotechnology (NY) 10:779-83 (1991) and the preparation of human mAbs (Boemer et al., J Immunol 147(1):86-95 (1991); Reisfeld and Sell, Monoclonal Antibodies and Cancer Therapy Alan R. Liss, Inc., New York (1985). Similarly, introducing human Ig genes into transgenic animals in which the endogenous Ig genes have been partially or completely inactivated can be exploited to synthesize human Abs. Upon challenge, human antibody production is observed, which closely resembles that seen in humans in all respects, including gene rearrangement, assembly, and antibody repertoire (U.S. Pat. No. 5,545,807, 1996; U.S. Pat. No. 5,545,806, 1996; U.S. Pat. No. 5,569,825, 1996; U.S. Pat. No. 5,633,425, 1997; U.S. Pat. No. 5,661,016, 1997; U.S. Pat. No. 5,625,126, 1997; Fishwild et al., Nat Biotechnol 14:845-51 (1996); Lonberg and Huszar, Int Rev Immunol 13:65-93 (1995); Lonberg et al., Nature 368:856-9 (1994); Marks et al., Biotechnology (NY) 10:779-783 (1992)).

In one preferred embodiment the instant inventions also comprises bi-specific mAbs that are monoclonal, preferably human or humanized, that have binding specificities for at least two different antigens. For example, a binding specificity is AMIGO; the other is for any antigen of choice, preferably a cell surface protein or receptor or receptor subunit.

Traditionally, the recombinant production of bi-specific Abs is based on the co-expression of two Ig heavy-chain/light-chain pairs, where the two heavy chains have different specificities (Milstein and Cuello, Nature 305:537-540 (1983)). Because of the random assortment of Ig heavy and light chains, the resulting hybridomas (quadromas) produce a potential mixture of ten different antibody molecules, of which only one has the desired bi-specific structure. The desired antibody can be purified using affinity chromatography or other techniques (WO 93/08829, (1993); Traunecker et al., Trends Biotechnol 9:109-113 (1991)).

To manufacture a bi-specific antibody (Suresh et al., Methods Enzymol. 121:210-228 (1986)), variable domains with the desired antibody-antigen combining sites are fused to Ig constant domain sequences. The fusion is preferably with an Ig heavy-chain constant domain, comprising at least part of the hinge, CH2, and CH3 regions. Preferably, the first heavy-chain constant region (CH1) containing the site necessary for light-chain binding is in at least one of the fusions. DNAs encoding the Ig heavy-chain fusions and, if desired, the Ig light chain, are inserted into separate expression vectors and are co-transfected into a suitable host organism.

Fab fragments may be directly recovered from E. coli and chemically coupled to form bi-specific Abs. For example, fully humanized bi-specific F(ab′) Abs can be produced (Shalaby et al., J Exp Med. 175:217-225 (1992)). Each Fab fragment is separately secreted from E. coli and directly coupled chemically in vitro, forming the bi-specific antibody.

Various techniques for making and isolating bi-specific antibody fragments directly from recombinant cell culture have also been described. For example, leucine zipper motifs can be exploited (Kostelny et al., Immunol. 148:1547-1553 (1992)). Peptides from the Fos and Jun proteins are linked to the Fab portions of two different Abs by gene fusion. The antibody homodimers are reduced at the hinge region to form monomers and then reoxidized to form antibody heterodimers. This method can also produce antibody homodimers.

The “diabody” technology (Holliger et al., Proc Natl Acad Sci USA. 90:6444-6448 (1993)) provides an alternative method to generate bi-specific antibody fragments. The fragments comprise a heavy-chain variable domain (VH) connected to a light-chain variable domain (VL) by a linker that is too short to allow pairing between the two domains on the same chain. The VH and VL domains of one fragment are forced to pair with the complementary VL and VH domains of another fragment, forming two antigen-binding sites. Another strategy for making bi-specific antibody fragments is the use of single-chain Fv (sFv) dimers (Gruber et al., Immunol. 152:5368-5374 (1994)). Abs with more than two valencies are also contemplated, such as tri-specific Abs (Tutt et al., J. Immunol. 147:60-69 (1991)).

Polyclonal Abs can be raised in a mammalian host, for example, by one or more injections of an immunogen and, if desired, an adjuvant. Typically, the immunogen and/or adjuvant are injected in the mammal by multiple subcutaneous or intraperitoneal injections. The immunogen may include AMIGO or an AMIGO fusion protein.

Examples of adjuvants include Freund's complete and monophosphoryl Lipid A synthetic-trehalose dicorynomycolate (MPL-TDM). To improve the immune response, an immunogen may be conjugated to a protein that is immunogenic in the AMIGO host, such as keyhole limpet hemocyanin (KLH), serum albumin, bovine thyroglobulin, and soybean trypsin inhibitor. Protocols for antibody production are described by (Harlow et al, supra). Alternatively, pAbs may be made in chickens, producing IgY molecules (Schade et al, The production of avian (egg yolg) antibodies: IgY. The report and recommendations of ECVAM workshop. Alternatives to Laboratory Animals NAILA). 24:925-934 (1996)).

Treatment

The AMIGO protein, AMIGO gene, and AMIGO nucleic acids are believed to find ex vivo or in vivo therapeutic use for administration to a mammal, particularly humans, in the treatment of diseases or disorders, related to AMIGO activity or benefited by AMIGO-responsiveness. Particularly preferred are neurologic disorders, preferably central nervous system disorders, Parkinson's disease, Alzheimer's disease, neuronal trauma or brain tumor.

The patient is administered an effective amount of AMIGO protein, biologically active peptide fragment, or variant of the invention or nucleic acids encoding said peptides. Therapeutic methods comprising administering AMIGO, AMIGO agonists, AMIGO antagonists or anti-AMIGO antibodies are within the scope of the present invention. The present invention also provides for pharmaceutical compositions comprising AMIGO protein, peptide fragment, or derivative in a suitable pharmacological carrier. The AMIGO protein, peptide fragment, or variant may be administered systemically or locally.

A disease or medical disorder is considered to be nerve damage if the survival or function of nerve cells and/or their axonal processes is compromised. Such nerve damage occurs as the result conditions including (a) Physical injury, which causes the degeneration of the axonal processes and/or nerve cell bodies near the site of the injury; (b) Ischemia, as a stroke; (c) Exposure to neurotoxins, such as the cancer and AIDS chemotherapeutic agents such as cisplatin and dideoxycytidine (ddC), respectively; (d) Chronic metabolic diseases, such as diabetes or renal dysfunction; and (e) Neurodegenerative diseases such as Parkinson's disease, Alzheimer's disease, and Amyotrophic Lateral Sclerosis (ALS), which cause the degeneration of specific neuronal populations. Conditions involving nerve damage include Parkinson's disease, Alzheimer's disease, Amyotrophic Lateral Sclerosis, stroke, diabetic polyneuropathy, toxic neuropathy, glial scar, and physical damage to the nervous system such as that caused by physical injury of the brain and spinal cord or crush or cut injuries to the arm and hand or other parts of the body, including temporary or permanent cessation of blood flow to parts of the nervous system, as in stroke.

The invention features a method for treating a mammal who has suffered an injury to the central nervous system, such as stroke or a traumatic injury. The method involves administering an AMIGO protein, peptide fragment, or variant of the invention to the affected mammal at least six hours after onset of the injury; for example twelve, twenty-four, forty-eight hours, or even longer following injury. No practical end point the therapeutic window in which the invention can be practiced has yet been established. The invention can be used to treat one or more adverse consequences of central nervous system injury that arise from a variety of conditions. Thrombus, embolus, and systemic hypotension are among the most common causes of stroke. Other injuries may be caused by hypertension, hypertensive cerebral vascular disease, rupture of an aneurysm, an angioma, blood dyscrasia, cardiac failure, cardiac arrest, cardiogenic shock, kidney failure, septic shock, head trauma, spinal cord trauma, seizure, bleeding from a tumor, or other loss of blood volume or pressure. These injuries lead to disruption of physiologic function, subsequent death of neurons, and necrosis (infarction) of the affected areas. The term “stroke” connotes the resulting sudden and dramatic neurologic deficits associated with any of the foregoing injuries.

The terms “ischemia” or “ischemic episode,” as used herein, mean any circumstance that results in a deficient supply of blood to a tissue. Thus, a central nervous system ischemic episode results from an insufficiency or interruption in the blood supply to any locus of the brain such as, but not limited to, a locus of the cerebrum, cerebellum or brain stem. The spinal cord, which is also a part of the central nervous system, is equally susceptible to ischemia resulting from diminished blood flow. An ischemic episode may be caused by a constriction or obstruction of a blood vessel, as occurs in the case of a thrombus or embolus. Alternatively, the ischemic episode may result from any form of compromised cardiac function, including cardiac arrest, as described above. Where the deficiency is sufficiently severe and prolonged, it can lead to disruption of physiologic function, subsequent death of neurons, and necrosis (infarction) of the affected areas. The extent and type of neurologic abnormality resulting from the injury depend on the location and size of the infarct or the focus of ischemia. Where the ischemia is associated with a stroke, it can be either global or focal in extent.

It is expected that the invention will also be useful for treating traumatic injuries to the central nervous system that are caused by mechanical forces, such as a blow to the head. Trauma can involve a tissue insult selected from abrasion, incision, contusion, puncture, compression, etc., such as can arise from traumatic contact of a foreign object with any locus of or appurtenant to the mammalian head, neck or vertebral column. Other forms of traumatic injury can arise from constriction or compression of mammalian CNS tissue by an inappropriate accumulation of fluid (e.g., a blockade or dysfunction of normal cerebrospinal fluid or vitreous humor fluid production, turnover or volume regulation, or a subdural or intracranial hematoma or edema). Similarly, traumatic constriction or compression can arise from the presence of a mass of abnormal tissue, such as a metastatic or primary tumor.

It is expected that the invention will also be useful for treating tumors or metastatic tumor cells, especially brain tumors. The most common brain tumors are gliomas, which begin in the glial tissue. Astrocytomas arise from small, star-shaped cells called astrocytes. In adults, astrocytomas most often arise in the cerebrum. A grade III astrocytoma is sometimes called anaplastic astrocytoma. A grade IV astrocytoma is usually called glioblastoma multiforme. Brain stem gliomas occur in the lowest, stemlike part of the brain. The brain stem controls many vital functions. Most brain stem gliomas are high-grade astrocytomas. Ependymomas usually develop in the lining of the ventricles. They may also occur in the spinal cord. Oligodendrogliomas arise in the cells that produce myelin, the fatty covering that protects nerves. These tumors usually arise in the cerebrum. They grow slowly and usually do not spread into surrounding brain tissue. Medulloblastomas develop from primitive nerve cells that normally do not remain in the body after birth. For this reason, medulloblastomas are sometimes called primitive neuroectodermal tumors (PNET). Most medulloblastomas arise in the cerebellum; however, they may occur in other areas as well. Meningiomas grow from the meninges. They are usually benign. Because these tumors grow very slowly, the brain may be able to adjust to their presence; meningiomas often grow quite large before they cause symptoms. They occur most often in women between 30 and 50 years of age. Schwannomas are benign tumors that begin in Schwann cells, which produce the myelin that protects the acoustic nerve. Acoustic neuromas are a type of schwannoma. Craniopharyngiomas develop in the region of the pituitary gland near the hypothalamus. They are usually benign; however, they are sometimes considered malignant because they can press on or damage the hypothalamus and affect vital functions. Germ cell tumors arise from primitive (developing) sex cells, or germ cells. The most frequent type of germ cell tumor in the brain is the germinoma. Pineal region tumors occur in or around the pineal gland. The tumor can be slow growing pineocytoma or fast growing (pineoblastoma). The pineal region is very difficult to reach, and these tumors often cannot be removed. Treatment for a brain tumor depends on a number of factors. Among these are the type, location, and size of the tumor, as well as the patient's age and general health. Normally brain tumors are treated with surgery, radiation therapy, and chemotherapy. Preferred tumours amenable for AMIGO treatment express EGFR and are responsive to AMIGO mediated inhibition of EGFR phosphorylation.

The invention is suitable for the treatment of any primate, preferably a higher primate such as a human. In addition, however, the invention may be employed in the treatment of domesticated mammals which are maintained as human companions (e.g., dogs, cats, horses), which have significant commercial value (e.g., goats, pigs, sheep, cattle, sporting or draft animals), which have significant scientific value (e.g., captive or free specimens of endangered species, or inbred or engineered animal strains), or which otherwise have value. One of ordinary skill in the medical or veterinary arts is trained to recognize whether a mammal is afflicted with an ischemic or traumatic injury of the central nervous system. For example, routine testing and/or clinical or veterinary diagnostic evaluation will reveal whether the mammal has acquired an impairment or loss of central nervous system (e.g., neurologic) function. Clinical and non-clinical indications, as well as accumulated experience, relating to the presently disclosed and other methods of treatment, should appropriately inform the skilled practitioner in deciding whether a given individual is afflicted with an ischemic or traumatic injury of the central nervous system and whether any particular treatment is best suited to the subject's needs, including treatment according to the present invention.

In gene therapy applications, genes are introduced into cells in order to achieve in vivo synthesis of a therapeutically effective genetic product, for example for replacement of a defective gene. “Gene therapy” includes both conventional gene therapy where a lasting effect is achieved by a single treatment, and the administration of gene therapeutic agents, which involves the one time or repeated administration of a therapeutically effective DNA or mRNA. Antisense RNAs and DNAs can be used as therapeutic agents for blocking the expression of certain genes in vivo. It has already been shown that short antisense oligonucleotides can be imported into cells where they act as inhibitors, despite their low intracellular concentrations caused by their restricted uptake by the cell membrane. (Zamecnik et al., Proc. Natl. Acad. Sci. USA, 83:41434146 (1986)). The oligonucleotides can be modified to enhance their uptake, e.g., by substituting their negatively charged phosphodiester groups by uncharged groups.

Another technique for inhibiting the expression of a gene involves the use of RNA for induction of RNA interference (RNAi), using double stranded (dsRNA) (Fire et al., Nature 391: 806-811. 1998) or short-interfering RNA (siRNA) sequences (Yu et al., Proc Natl Acad Sci USA. 99:6047-52. 2002). “RNAi” is the process by which dsRNA induces homology-dependent degradation of complimentary mRNA. In one embodiment, a synthetic antisense nucleic acid molecule is hybridized by complementary base pairing with a “sense” ribonucleic acid to form a double stranded RNA. The dsRNA antisense and sense nucleic acid molecules are provided that correspond to at least about 20, 25, 50, 100, 250 or 500 nucleotides or an entire AMIGO coding strand, or to only a portion thereof. In an alternative embodiment, the siRNAs are 30 nucleotides or less in length, and more preferably 21- to 23-nucleotides, with characteristic 2- to 3-nucleotide 3′-overhanging ends, which are generated by ribonuclease III cleavage from longer dsRNAs. (See e.g. Tuschl T. Nat Biotechnol. 20:446-48. 2002).

Intracellular transcription of small RNA molecules can be achieved by cloning the siRNA templates into RNA polymerase III (Pol III) transcription units, which normally encode the small nuclear RNA (snRNA) U6 or the human RNAse P RNA H1. Two approaches can be used to express siRNAs: in one embodiment, sense and antisense strands constituting the siRNA duplex are transcribed using constructs with individual promoters (Lee, et al. Nat. Biotechnol. 20, 500-505. 2002); in an alternative embodiment, siRNAs are expressed as stem-loop hairpin RNA structures that give rise to siRNAs after intracellular processing (Brummelkamp et al. Science 296:550-553. 2002) (herein incorporated by reference).

The dsRNA/siRNA is most commonly administered by annealing sense and antisense RNA strands in vitro before delivery to the organism. In an alternate embodiment, RNAi may be carried out by administering sense and antisense nucleic acids of the invention in the same solution without annealing prior to administration, and may even be performed by administering the nucleic acids in separate vehicles within a very close timeframe. Nucleic acid molecules encoding fragments and variants of an AMIGO or antisense nucleic acids complementary to an AMIGO nucleic acid sequence are additionally provided.

There are a variety of techniques available for introducing nucleic acids into viable cells. The techniques vary depending upon whether the nucleic acid is transferred into cultured cells in vitro, ex vivo, or in vivo in the cells of the intended host. Techniques suitable for the transfer of nucleic acid into mammalian cells in vitro include the use of liposomes, (Nicolau and Sene, Biochim. Biophys. Acta, 721:185-190 (1982); Fraley, et al., Proc. Natl. Acad. Sci. USA, 76:3348-3352 (1979); Feigner, Sci. Am., 276(6):102-6 (1997); Feigner, Hum. Gene Ther., 7(15): 1791-3, (1996)), electroporation (Tur-Kaspa, et al., Mol. Cell Biol., 6:716-718, (1986); Potter, et al., Proc. Nat. Acad. Sci. USA, 81:7161-7165, (1984)), direct microinjection (Harland and Weintraub, J. Cell Biol., 101:1094-1099 (1985)), cell fusion, DEAE-dextran (Gopal, Mol. Cell Biol., 5:1188-1190 (1985), the calcium phosphate precipitation method (Graham and Van Der Eb, Virology, 52:456-467 (1973); Chen and Okayama, Mol. Cell Biol., 7:2745-2752, (1987); Rippe, et al., Mol. Cell Biol., 10:689-695 (1990), cell sonication (Fechheimer, et al., Proc. Natl. Acad. Sci. USA, 84:8463-8467 (1987)), gene bombardment using high velocity microprojectiles (Yang, et al., Proc. Natl. Acad. Sci. USA, 87:9568-9572 (1990). The currently preferred in vivo gene transfer techniques include transfection with viral (typically retroviral) vectors and viral coat protein-liposome mediated transfection (Dzau et al., Trends in Biotechnology, 11:205-210 (1993)). In some situations it is desirable to provide the nucleic acid source with an agent that targets the target cells, such as an antibody specific for a cell surface membrane protein or the target cell, a ligand for a receptor on the target cell, etc. Where liposomes are employed, proteins which bind to a cell surface membrane protein associated with endocytosis may be used for targeting and/or to facilitate uptake, e.g. capsid proteins or fragments thereof tropic for a particular cell type, antibodies for proteins which undergo internalization in cycling, and proteins that target intracellular localization and enhance intracellular half-life. The technique of receptor-mediated endocytosis is described, for example, by Wu et al., J. Biol. Chem., 262:4429-4432 (1987); and Wagner et al., Proc. Natl. Acad. Sci. USA, 87:3410-3414 (1990). For review of the currently known gene marking and gene therapy protocols see Anderson et al., Science, 256:808-813 (1992).

Any suitable vector may be used to introduce a transgene of interest into an animal. Exemplary vectors that have been described in the literature include replication-deficient retroviral vectors, including but not limited to lentivirus vectors [Kim et al., J. Virol., 72(1): 811-816 (1998); Kingsman & Johnson, Scrip Magazine, October, 1998, pp. 43-46.]; adenoviral (see, for example, U.S. Pat. No. 5,824,544; U.S. Pat. No. 5,707,618; U.S. Pat. No. 5,792,453; U.S. Pat. No. 5,693,509; U.S. Pat. No. 5,670,488; U.S. Pat. No. 5,585,362; Quantin et al., Proc. Natl. Acad. Sci. USA, 89: 2581-2584 (1992); Stratford-Perricadet et al., J. Clin. Invest., 90: 626-630 (1992); and Rosenfeld et al., Cell, 68: 143-155 (1992)), retroviral (see, for example, U.S. Pat. No. 5,888,502; U.S. Pat. No. 5,830,725; U.S. Pat. No. 5,770,414; U.S. Pat. No. 5,686,278; U.S. Pat. No. 4,861,719), adeno-associated viral (see, for example, U.S. Pat. No. 5,474,935; U.S. Pat. No. 5,139,941; U.S. Pat. No. 5,622,856; U.S. Pat. No. 5,658,776; U.S. Pat. No. 5,773,289; U.S. Pat. No. 5,789,390; U.S. Pat. No. 5,834,441; U.S. Pat. No. 5,863,541; U.S. Pat. No. 5,851,521; U.S. Pat. No. 5,252,479; Gnatenko et al., J. Investig. Med., 45: 87-98 (1997), an adenoviral-adenoassociated viral hybrid (see, for example, U.S. Pat. No. 5,856,152) or a vaccinia viral or a herpesviral (see, for example, U.S. Pat. No. 5,879,934; U.S. Pat. No. 5,849,571; U.S. Pat. No. 5,830,727; U.S. Pat. No. 5,661,033; U.S. Pat. No. 5,328,688); Lipofectin-mediated gene transfer (BRL); liposomal vectors [See, e.g., U.S. Pat. No. 5,631,237 (Liposomes comprising Sendai virus proteins)]; and combinations thereof. All of the foregoing documents are incorporated herein by reference in the entirety. Replication-deficient adenoviral vectors, adeno-associated viral vectors and lentiviruses constitute preferred embodiments.

In embodiments employing a viral vector, preferred polynucleotides include a suitable promoter and polyadenylation sequence to promote expression in the target tissue of interest. For many applications of the present invention, suitable promoters/enhancers for mammalian cell expression include, e.g., cytomegalovirus promoter/enhancer [Lehner et al., J. Clin. Microbiol., 29:2494-2502 (1991); Boshart et al., Cell, 41:521-530 (1985)]; Rous sarcoma virus promoter [Davis et al., Hum. Gene Ther., 4:151 (1993)]; simian virus 40 promoter, long terminal repeat (LTR) of retroviruses, keratin 14 promoter, and α myosin heavy chain promoter.

In a particular embodiment of the invention, the expression construct (or the peptides discussed above) may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, “In Liver Diseases, Targeted Diagnosis And Therapy Using Specific Receptors And Ligands,” Wu, G., Wu, C., ed., New York: Marcel Dekker, pp. 87-104 (1991)). The addition of DNA to cationic liposomes causes a topological transition from liposomes to optically birefringent liquid-crystalline condensed globules (Radler, et al., Science, 275(5301):810-4, (1997)). These DNA-lipid complexes are potential non-viral vectors for use in gene therapy and delivery.

Also contemplated in the present invention are various commercial approaches involving “lipofection” technology. In certain embodiments of the invention, the liposome may be complexed with a hemagglutinating virus (HVJ). This has been shown to facilitate fusion with the cell membrane and promote cell entry of liposome-encapsulated DNA (Kaneda, et al., Science, 243:375-378 (1989)). In other embodiments, the liposome may be complexed or employed in conjunction with nuclear nonhistone chromosomal proteins (HMG-1) (Kato, et al., J. Biol. Chem., 266:3361-3364 (1991)). In yet further embodiments, the liposome may be complexed or employed in conjunction with both HVJ and HMG-1. In that such expression constructs have been successfully employed in transfer and expression of nucleic acid in vitro and in vivo, then they are applicable for the present invention.

Other vector delivery systems that can be employed to deliver a nucleic acid encoding a therapeutic gene into cells include receptor-mediated delivery vehicles. These take advantage of the selective uptake of macromolecules by receptor-mediated endocytosis in almost all eukaryotic cells. Because of the cell type-specific distribution of various receptors, the delivery can be highly specific (Wu and Wu (1993), supra).

In another embodiment of the invention, the expression construct may simply consist of naked recombinant DNA or plasmids. Transfer of the construct may be performed by any of the methods mentioned above that physically or chemically permeabilize the cell membrane. This is applicable particularly for transfer in vitro, however, it may be applied for in vivo use as well. Dubensky, et al., Proc. Nat. Acad. Sci. USA, 81:7529-7533 (1984) successfully injected polyomavirus DNA in the form of CaPO₄ precipitates into liver and spleen of adult and newborn mice demonstrating active viral replication and acute infection. Benvenisty and Neshif, Proc. Nat. Acad. Sci. USA, 83:9551-9555 (1986) also demonstrated that direct intraperitoneal injection of CaPO₄ precipitated plasmids results in expression of the transfected genes.

Another embodiment of the invention for transferring a naked DNA expression construct into cells may involve particle bombardment. This method depends on the ability to accelerate DNA coated microprojectiles to a high velocity allowing them to pierce cell membranes and enter cells without killing them (Klein, et al., Nature, 327:70-73 (1987)). Several devices for accelerating small particles have been developed. One such device relies on a high voltage discharge to generate an electrical current, which in turn provides the motive force (Yang, et al., Proc. Natl. Acad. Sci USA, 87:9568-9572 (1990)). The microprojectiles used have consisted of biologically inert substances such as tungsten or gold beads.

Those of skill in the art are aware of how to apply gene delivery to in vivo and ex vivo situations. For viral vectors, one generally will prepare a viral vector stock. Depending on the type of virus and the titer attainable, one will deliver 1×10⁴, 1×10⁵, 1×10⁶, 1×10⁷, 1×10⁸, 1×10⁹, 1×10¹⁰, 1×10¹¹ or 1×10¹² infectious particles to the patient. Similar figures may be extrapolated for liposomal or other non-viral formulations by comparing relative uptake efficiencies. Formulation as a pharmaceutically acceptable composition is discussed below.

Various routes are contemplated for various cell types. For practically any cell, tissue or organ type, systemic delivery is contemplated. In other embodiments, a variety of direct, local and regional approaches may be taken. For example, the cell, tissue or organ may be directly injected with the expression vector or protein.

In a different embodiment, ex vivo gene therapy is contemplated. In an ex vivo embodiment, cells from the patient are removed and maintained outside the body for at least some period of time. During this period, a therapy is delivered, after which the cells are reintroduced into the patient.

The invention also provides antagonists of AMIGO activation (e.g., AMIGO antisense nucleic acid, RNAi, neutralizing antibodies). Administration of AMIGO antagonist to a mammal having increased or excessive levels of endogenous AMIGO activation is contemplated, preferably in the situation where such increased levels of AMIGO lead to a pathological disorder.

Pharmaceutical and Therapeutical Compositions and Formulations

The AMIGO nucleic acid molecules, AMIGO polypeptides, AMIGO agonists, AMIGO antagonists and anti-AMIGO Abs (active compounds) of the invention, and derivatives, fragments, analogs and homologs thereof, can be incorporated into pharmaceutical compositions.

Such compositions of AMIGO are prepared for storage by mixing AMIGO nucleic acid molecule, protein, or antibody having the desired degree of purity with optional physiologically acceptable carriers, excipients, or stabilizers (Remington's Pharmaceutical Sciences, 16th edition, Osol, A., Ed., (1980)), in the form of lyophilized cake or aqueous solutions. Acceptable carriers, excipients, or stabilizers are non-toxic to recipients at the dosages and concentrations employed, and include buffers such as phosphate, citrate, and other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine, or lysine; monosaccharides, disaccharides, and other carbohydrates including glucose, mannose, or dextrins; chelating agents such as EDTA; sugar alcohols such as mannitol or sorbitol; salt-forming counter-ions such as sodium; and/or non-ionic surfactants such as Tween, Pluronics or polyethylene glycol (PEG).

The AMIGO nucleic acid molecule, protein, agonist, antagonist or antibodies may also be entrapped in microcapsules prepared, for example, by coacervation techniques or by interfacial polymerization (for example, hydroxymethylcellulose or gelatin-microcapsules and poly-(methylmethacylate) microcapsules, respectively), in colloidal drug delivery systems (for example, liposomes, albumin microspheres, microemulsions, nano-particles, and nanocapsules), or in macroemulsions. Such techniques are disclosed in Remington's Pharmaceutical Sciences, supra.

The route of AMIGO nucleic acid molecule, protein, or antibody administration is in accord with known methods, e.g., those routes set forth above for specific indications, as well as the general routes of injection or infusion by intravenous, intraperitoneal, intracerebral, intramuscular, intraocular, intrathecal, intracranial, intraspinal, intraventricular, intraarterial, or intralesional means, or sustained release systems as noted below. AMIGO nucleic acid molecule, protein, or antibody is administered continuously by infusion or by bolus injection. Generally, where the disorder permits, one should formulate and dose the AMIGO nucleic acid molecule, protein, or antibody for site-specific delivery. Administration can be continuous or periodic. Administration can be accomplished by a constant- or programmable-flow implantable pump or by periodic injections. The nucleic acid molecules of the invention can be inserted into vectors and used as gene therapy vectors. Gene therapy vectors can be delivered to a subject by, for example, intravenous injection, local administration (Nabel and Nabel, U.S. Pat. No. 5,328,470, 1994), or by stereotactic injection (Chen et al., Proc. Natl. Acad. Sci. USA 91:3054-3057 (1994)). The pharmaceutical preparation of a gene therapy vector can include an acceptable diluent, or can comprise a slow release matrix in which the gene delivery vehicle is imbedded.

Alternatively, where the complete gene delivery vector can be produced intact from recombinant cells, e.g., retroviral vectors, the pharmaceutical preparation can include one or more cells that produce the gene delivery system.

Suitable examples of sustained-release preparations include semipermeable matrices of solid hydrophobic polymers containing the protein, which matrices are in the form of shaped articles, e.g., films, or microcapsules. Examples of sustained-release matrices include polyesters, hydrogels as described by Langer et al., J. Biomed. Mater. Res., 15:167-277 (1981) and Langer, Chem. Tech., 12:98-105 (1982) or polyvinylalcohol, polylactides (U.S. Pat. No. 3,773,919, EP 58,481), or non-degradable ethylene-vinyl acetate (Langer et al., supra).

Sustained-release AMIGO compositions also include liposomally entrapped AMIGO nucleic acid molecule, protein, agonist, antagonist or antibodies. Liposomes containing AMIGO nucleic acid molecule, protein, or antibodies are prepared by methods known per se: Epstein et al., Proc. Natl. Acad. Sci. USA, 82:3688-3692 (1985); Hwang et al., Proc. Natl. Acad. Sci. USA, 77:40304034 (1980); EP 52,322; EP 36,676; EP 88,046; EP 143,949; EP 142,641; U.S. Pat. Nos. 4,485,045 and 4,544,545; and EP 102,324. Ordinarily the liposomes are of the small (about 200-800 Angstroms) unilamellar type in which the lipid content is greater than about 30 mol % cholesterol, the selected proportion being adjusted for the optimal AMIGO nucleic acid molecule, protein, or antibody therapy.

While polymers such as ethylene-vinyl acetate and lactic acid-glycolic acid enable release of molecules for over 100 days, certain hydrogels release proteins for shorter time periods. When encapsulated proteins remain in the body for a long time, they may denature or aggregate as a result of exposure to moisture at 37° C., resulting in a loss of biological activity and possible changes in immunogenicity. Rational strategies can be devised for protein stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

Semipermeable, implantable membrane devices are useful as means for delivering drugs in certain circumstances. For example, cells that secrete soluble AMIGO or express AMIGO on their cell surface, chimeras or antibodies can be encapsulated, and such devices can be implanted into a patient. For example, into the brain of patients suffering from Parkinson's Disease, neuronal trauma or glial scar. See, U.S. Pat. No. 4,892,538 of Aebischer et al.; U.S. Pat. No. 5,011,472 of Aebischer et al.; U.S. Pat. No. 5,106,627 of Aebischer et al.; PCT Application WO 91/10425; PCT Application WO 91/10470; Winn et al., Exper. Neurology, 113:322-329 (1991); Aebischer et al., Exper Neurology, 111:269-275 (1991); and Tresco et al., ASAIO, 38:17-23 (1992).

Accordingly, also included is a method for preventing or treating damage to a nerve or damage to other AMIGO-responsive cells, which comprises implanting cells that secrete AMIGO or express AMIGO on their cell surface, its agonists or antagonists as may be required for the particular condition, into the body of patients in need thereof. Finally, the present invention includes a device for preventing or treating nerve damage or damage to other cells as taught herein by implantation into a patient comprising a semipermeable membrane, and a cell that secretes AMIGO (or its agonists or antagonists as may be required for the particular condition) encapsulated within said membrane and said membrane being permeable to AMIGO (or its agonists or antagonists) and impermeable to factors from the patient detrimental to the cells. The patient's own cells, transformed to produce AMIGO ex vivo, could be implanted directly into the patient, optionally without such encapsulation. The methodology for the membrane encapsulation of living cells is familiar to those of ordinary skill in the art, and the preparation of the encapsulated cells and their implantation in patients may be accomplished without under experimentation.

The present invention includes, therefore, a method for preventing or treating nerve damage by implanting cells, into the body of a patient in need thereof, cells either selected for their natural ability to generate or engineered to secrete AMIGO or AMIGO antibody. Preferably, the expressed or secreted AMIGO or antibody being soluble, human mature AMIGO when the patient is human. The implants are preferably non-immunogenic and/or prevent immunogenic implanted cells from being recognized by the immune system. For CNS delivery, a preferred location for the implant is the cerebral spinal fluid of the spinal cord.

An effective amount of AMIGO nucleic acid molecule, protein, agonist, antagonist or antibody to be employed therapeutically will depend, for example, upon the therapeutic objectives, the route of administration, and the condition of the patient. Accordingly, it will be necessary for the therapist to titre the dosage and modify the route of administration as required to obtain the optimal therapeutic effect. Typically, the clinician will administer the AMIGO protein or antibody until a dosage is reached that achieves the desired effect. A typical daily dosage for systemic treatment might range from about 1 microgram/kg to up to 10 mg/kg or more, depending on the factors mentioned above. As an alternative general proposition, the AMIGO nucleic acid molecule, protein, or antibody is formulated and delivered to the target site or tissue at a dosage capable of establishing in the tissue an AMIGO level that is efficacious but not unduly toxic. This intra-tissue concentration should be maintained if possible by continuous infusion, sustained release, topical application, AMIGO-expressing cell implant, or injection at empirically determined frequencies. The progress of this therapy is easily monitored by conventional assays.

As will be appreciated by one of ordinary skill in the art, the formulated compositions contain therapeutically-effective amounts of the AMIGO protein, peptide fragment, or variant of the invention or modulator of AMIGO receptors. That is, they contain an amount which provides appropriate concentrations of the agent to the affected nervous system tissue for a time sufficient to stimulate a detectable restoration of central nervous system function, up to and including a complete restoration thereof. As will be appreciated by those skilled in the art, these concentrations will vary depending upon a number of factors, including the biological efficacy of the selected agent, the chemical characteristics (e.g., hydrophobicity) of the specific agent, the formulation thereof, including a mixture with one or more excipients, the administration route, and the treatment envisioned, including whether the active ingredient will be administered directly into a tissue site, or whether it will be administered systemically. The preferred dosage to be administered also is likely to depend on such variables such as the condition of the diseased or damaged tissues, and the overall health status of the particular mammal. As a general matter, single, daily, biweekly or weekly dosages of 0.00001-1000 mg of an AMIGO protein, peptide fragment, or variant of the invention or agonists of AMIGO receptors are sufficient with 0.0001-100 mg being preferable, and 0.001 to 10 mg being even more preferable. Alternatively, a single, daily, biweekly or weekly dosage of 0.01-1000.mu·g/kg body weight, more preferably 0.01-10 mg/kg body weight, may be advantageously employed. The present effective dose can be administered in a single dose or in a plurality (two or more) of installment doses, as desired or considered appropriate under the specific circumstances. A bolus injection or diffusable infusion formulation can be used. If desired to facilitate repeated or frequent infusions, implantation of a semi-permanent stent (e.g., intravenous, intraperitoneal, intracisternal or intracapsular) may be advisable. In Example below, intraspinal administration of AMIGO, AMIGO2 or AMIGO3 confer clearly detectable levels of restoration of lost or impaired central nervous system function.

Uses of AMIGO Compounds

The present invention employs AMIGO compounds for use in inhibiting the function of EGFR, ultimately modulating the phosphorylation of EGFR and thus modulating the signalling cascade initiated by EGFR. This is accomplished by providing AMIGO compounds which specifically bind and modulate EGFR phosphorylation. Such AMIGO compounds interfere with the normal role of EGFR function and causes a modulation of its cellular signaling. The functions of EGFR phosphorylation to be interfered include all vital functions such as, for example, ligand-receptor interaction, dimerization of EGFR in the cell membrane, phosphorylation of EGFR, modulation of EGFR initiated signalling cascades which may be engaged in by EGFR. The overall effect of such interference with AMIGO compounds is modulation of the phosphorylation of EGFR. In the context of this invention, “modulation” means either an increase (stimulation) or a decrease (inhibition) in the phosphorylation of an EGFR. In the context of the present invention, inhibition is the preferred form of modulation of EGFR phosphorylation.

Some of the featured AMIGO compounds can be used to treat cell proliferative disorders characterized by inappropriate EGFR activity. “Inappropriate EGFR” activity refers to either: 1) EGF-receptor (EGFR) expression in cells which normally do not express EGFR; 2) EGF expression by cells which normally do not express EGF/TGF-.alpha.; 3) increased EGF-receptor (EGFR) expression leading to unwanted cell proliferation; 4) increased EGF/TGF-.alpha. expression leading to unwanted cell proliferation; and/or 5) mutations leading to constitutive activation of EGF-receptor (EGFR). The existence of inappropriate or abnormal EGF/TGF-.alpha. and EGFR levels or activities is determined by procedures well known in the art.

An increase in EGF/TGF-.alpha. activity or expression is characterized by an increase in one or more of the activities which can occur upon EGF ligand binding such as: (I) EGF-R dimerization; (2) auto-phosphorylation of EGFR, (3) phosphorylation of an EGFR substrate (e.g., PLC.gamma.), (4) activation of an adapter molecule, and/or (5) increased cell division. These activities can be measured using techniques described below and known in the art. For example auto-phosphorylation of EGFR can be measured using an anti-phosphotyrosine antibody, and increased cell division can be performed by measuring .sup.3H-thymidine incorporation into DNA. Preferably, the increase in EGFR activity is characterized by an increased amount of phosphorylated EGFR and/or DNA synthesis.

Unwanted cell proliferation can result from inappropriate EGFR activity occurring in different types of cells including cancer cells, cells surrounding a cancer cell, and endothelial cells. Examples of disorders characterized by inappropriate EGF activity include cancers such as glioma, head, neck, gastric, lung, breast, ovarian, colon, and prostate.

AMIGO compound. The term “AMIGO compound” is meant to refer to an AMIGO peptide, variants, biologically active fragments, antigenic fragment, anti-AMIGO antibodies or binding portion thereof and nucleic acids encoding said peptides which are capable of binding to or interacting in some way with EGFR or a ligand of the epidermal growth factor receptor. Binding or interaction of an AMIGO compound of the invention with the corresponding ligand results in the modulation, preferably prevention or inhibition, of the interaction between a ligand and its corresponding receptor. Because the ligand-receptor interaction is involved in the proliferation of EGFR-expressing tumour cells, the term “an AMIGO compound” is meant to include all compounds which modulate the interaction between the epidermal growth factor receptor and their corresponding ligands, more preferably adversely affect interaction between the epidermal growth factor receptor and their corresponding ligands leading to inhibition of phosphorylation of EGFR.

As used herein, the terms “inhibits phosphorylation” (e.g., referring to inhibition/blocking of phosphorylation of EGFR) encompass both partial and complete inhibition. The inhibition of EGFR phosphorylation preferably reduces or alters the normal level or type of cell signaling that occurs when EGFR ligand binds to EGFR without inhibition or blocking. Inhibition is also intended to include any measurable decrease in the binding affinity of EGFR ligand to EGFR when in contact with an AMIGO compound as compared to the ligand not in contact with an AMIGO compound, e.g., the blocking of EGFR ligands to EGFR by at least about 10%, 20%, 30%, 40%, 50%, 60%, 70%, 80%, 90%, 99%, or 100%.

The AMIGO compounds of the present invention have a multitude of therapeutic and diagnostic uses. For example, therapeutic uses involve cancer therapy in a patient suspected of suffering from cancer or other related diseases. Specifically, AMIGO compounds of the present invention can be used to treat patients that have tumour cells which produce the EGFR ligand and/or overexpress the EGFR proteins.

One type of treatment may involve the use of the AMIGO compounds coupled to a therapeutic agent. By administering an effective amount of AMIGO compounds coupled with the therapeutic agent to a patient, a tumour cells in the patient which express EGFR can be growth inhibited or killed, thereby providing a treatment for cancer.

In accordance with the method of cancer treatment of the invention, the conjugated AMIGO compound is capable of recognizing and binding to tumour cells due to the association of the tumour cells with the EGFR. Without being limited, the mechanism of binding to the cancer cell may involve the recognition of EGFR, ligand located on the cell surface or because of expression and/or secretion of the ligand.

Once the conjugated AMIGO compound is bound or in close association with the tumour cell by interacting with EGFR, the therapeutic agent is capable of inhibiting or killing that cell. In this manner, the therapy of the present invention is selective for a particular target, e.g., cancer cells which are associated with the EGFR.

Normal cells and other cells not associated with the EGFR (cells which do not express EGFR) may not, for the most part, be affected by therapy with AMIGO compounds.

Alternatively, the AMIGO compounds of the present invention may be used to prevent or inhibit inducement of tumour cell proliferation. For example, cancer cells which contain the EGFR are induced to proliferate in the presence of low concentrations of EGFR ligand. Preventing the EGFR ligand from interacting with its receptor may provide a means to treat a cancer patient.

According to the method of inhibiting or preventing cellular proliferation of the present invention, the AMIGO compound is capable of binding to the EGFR. Binding the EGFR in vivo forms an EGFR-AMIGO compound complex and thus may prevent or inhibit the ligand-receptor interaction either sterically or otherwise. Thus, the present invention provides a treatment to prevent or inhibit tumour cell proliferation in a patient by administering an effective amount of an AMIGO compound to such a patient.

It will be appreciated that a number of other therapeutic uses of the AMIGO compounds of the present invention may be devised. Such therapies may involve use of other known treatment techniques in combination with the AMIGO compounds of the invention. The present invention is not meant to be limited to the therapeutic treatment described and are thus only presented by way of illustration.

Furthermore, administration of an amount of the AMIGO compounds of the present invention sufficient to inhibit or kill a tumour cell may vary depending upon a number of factors including the type of malignant cell, body weight of the patient, the type of therapeutic agent used and the like. Those of skill in the art will appreciate that the amount necessary to inhibit or kill a particular malignant cell in vitro or in vivo can easily be determined with minimal routine experimentation. An effective amount of such AMIGO compounds may be administered parenterally, subcutaneously, intravenously, intramuscularly, intraperitoneally or orally. In addition, pharmaceutical preparations may be prepared which contain suitable excipients, auxiliaries, or compounds which facilitate processing or stability of the AMIGO compounds of the invention as pharmaceutical agents.

Diagnostic uses of the AMIGO compounds of the present invention (due to its modification of EGFR phosphorylation) may include, for example, detection of EGFR in a sample obtained from a patient. Such samples may be body tissue, body fluids (such as blood, urine, tear drops, saliva, serum, and cerebrospinal fluid), feces, cellular extracts and the like.

Assaying for the EGFR phosphorylation of the invention in a sample obtained from a patient may thus provide for a method for diagnosing cancer. That is, detection of EGFR in a sample obtained from a patient indicates the presence of EGFR expressing cells in a patient. Furthermore, since the AMIGO compound is specific for, EGFR, the phosphorylation assay may provide information concerning the biology of a patient's tumor. For example, cancer patients with a tumour cells that overexpress the EGFR are known to have poorer overall survival than cancer patients that do not show EGFR overexpression. Detection of EGFR phosphorylation may thus serve as a prognostic test, allowing the clinician to select a more effective therapy for treating the patient.

The AMIGO compound compositions of the invention can be initially tested for binding activity associated with therapeutic or diagnostic use in vitro. For example, compositions of the invention can be tested using the ELISA and flow cytometric assays described in the Examples below. Moreover, the activity of these molecules in triggering at least one effector-mediated effector cell activity, including phosphorylation of EGFR of cells expressing EGFR can be assayed.

The compositions of the invention have additional utility in therapy and diagnosis of EGFR-related diseases. For example, the AMIGO DNA can be used to elicit in vivo or in vitro one or more of the following biological activities: to inhibit EGF or TGF-.alpha. induced autophosphorylation in a cell expressing EGFR; to inhibit autocrine EGF or TGF-.alpha.-induced activation of a cell expressing EGFR; or to inhibit the growth of a cell expressing EGFR, e.g., at low dosages.

In a particular embodiment, the AMIGO compounds and derivatives/variants thereof are used in vivo to treat, prevent or diagnose a variety of EGFR-related diseases. Examples of EGFR-related diseases include a variety of cancers, such as glioma, glioblastoma, bladder, breast, uterine/cervical, colon, pancreatic, colorectal, kidney, stomach, ovarian, prostate, renal cell, squamous cell, lung (non-small cell), esophageal, and head and neck cancer.

Methods of administering the compositions of the invention are known in the art. Suitable dosages of the molecules used will depend on the age and weight of the subject and the particular drug used. The AMIGO compounds can be coupled to radionuclides, such as 131I, 90Y, 105Rh, indium-111, etc., as described in Goldenberg, D. M. et al. (1981) Cancer Res. 41: 4354-4360, and in EP 0365 997. In another aspect the invention relates to an immunoconjugate comprising an AMIGO antibody or binding portion thereof or AMIGO peptide or fragment according to the invention linked to a radioisotope, cytotoxic agent (e.g., calicheamicin and duocarmycin), a cytostatic agent, or a chemotherapeutic drug. The compositions of the invention can also be coupled to anti-infectious agents.

In another embodiment, the AMIGO compounds can be co-administered with a therapeutic agent, e.g., a chemotherapeutic agent, an immunosuppressive agent, or can be co-administered with other known therapies, such as physical therapies, e.g., radiation therapy, hyperthermia, or transplantation (e.g., bone marrow transplantation). Such therapeutic agents include, among others, anti-neoplastic agents such as doxorubicin (adriamycin), cisplatin bleomycin sulfate, carmustine, chlorambucil, and cyclophosphamide hydroxyurea which, by themselves, are only effective at levels which are toxic or subtoxic to a patient. Cisplatin is intravenously administered as a 100 mg/m.sup.2 dose once every four weeks and adriamycin is intravenously administered as a 60-75 mg/m.sup.2 dose once every 21 days.

Pharmaceutical compositions of the present invention can include one or more further chemotherapeutic agents selected from the group consisting of nitrogen mustards (e.g., cyclophosphamide and ifosfamide), aziridines (e.g., thiotepa), alkyl sulfonates (e.g., busulfan), nitrosoureas (e.g., carmustine and streptozocin), platinum complexes (e.g., carboplatin and cisplatin), non-classical alkylating agents (e.g., dacarbazine and temozolamide), folate analogs (e.g., methotrexate), purine analogs (e.g., fludarabine and mercaptopurine), adenosine analogs (e.g., cladribine and pentostatin), pyrimidine analogs (e.g., fluorouracil (alone or in combination with leucovorin) and gemcitabine), substituted ureas (e.g., hydroxyurea), antitumor antibiotics (e.g., bleomycin and doxorubicin), epipodophyllotoxins (e.g., etoposide and teniposide), microtubule agents (e.g., docetaxel and paclitaxel), camptothecin analogs (e.g., irinotecan and topotecan), enzymes (e.g., asparaginase), cytokines (e.g., interleukin-2 and interferon-.alpha.), monoclonal antibodies (e.g., trastuzumab and bevacizumab), recombinant toxins and immunotoxins (e.g., recombinant cholera toxin-B and TP-38), cancer gene therapies, physical therapies (e.g., hyperthermia, radiation therapy, and surgery) and cancer vaccines (e.g., vaccine against telomerase).

Co-administration of the AMIGO compounds of the present invention with chemotherapeutic agents provides two anti-cancer agents which operate via different mechanisms which yield a cytotoxic effect to human tumor cells. Such co-administration can solve problems due to development of resistance to drugs or a change in the antigenicity of the tumor cells which would render them unreactive with the antibody.

In another embodiment, the subject can be additionally treated with a lymphokine preparation. Cancer cells which do not highly express EGFR can be induced to do so using lymphokine preparations. Lymphokine preparations can cause a more homogeneous expression of EGFRs among cells of a tumor which can lead to a more effective therapy.

Lymphokine preparations suitable for administration include interferon-gamma, tumor necrosis factor, and combinations thereof. These can be administered intravenously. Suitable dosages of lymphokine are 10,000 to 1,000,000 units/patient.

In one embodiment, the invention provides methods for detecting the presence of EGFR phosphorylation in a sample, or measuring the amount of EGFR phosphorylation, comprising contacting the sample, and a control sample, with an AMIGO compound, which specifically binds to EGFR, under conditions that allow for formation of a complex between the AMIGO compound and EGFR. The formation of a complex is then detected, i.e. modulation, preferably inhibition of phosphorylation, wherein a difference in EGFR phosphorylation between the sample compared to the control sample is indicative the presence of EGFR in the sample.

Screening

The present invention also encompasses agent which modulate AMIGO expression or activity. An agent may, for example, be a small molecule. For example, such small molecules include, but are not limited to, peptides, peptidomimetics (e.g., peptoids), amino acids, amino acid analogs, polynucleotides, polynucleotide analogs, nucleotides, nucleotide analogs, organic or inorganic compounds (i.e,. including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. It is understood that appropriate doses of small molecule agents depends upon a number of factors within the ken of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention. Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated. Such appropriate doses may be determined using the assays described herein. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

The subject methods include screens for agents which modulate homophilic or heterophilic AMIGO interactions and methods for modulating these interactions. AMIGO activation is found to regulate a wide variety of cell functions, including cell-cell interactions, cell mobility, neurite growth and fasciculation. AMIGO polypeptides are disclosed as specific modulators of function of EGFR polypeptides. Accordingly, the invention provides methods for modulating targeted cell function comprising the step of modulating AMIGO activation by contacting the cell with a modulator of a AMIGO:AMIGO or AMIGO:AMIGO ligand interaction. The invention also provides methods for modulating targeted cell function comprising the step of modulating EGFR activation by contacting the cell with a modulator of a AMIGO:EGFR interaction.

In another aspect, the invention provides methods of screening for agents which modulate AMIGO:AMIGO, AMIGO:EGFR or AMIGO:AMIGO ligand interactions. These methods generally involve forming a mixture of an AMIGO-expressing cell, an AMIGO, EGFR or AMIGO ligand polypeptide and a candidate agent, and determining the effect of the agent on the amount of AMIGO expressed by the cell. The methods are amenable to automated, cost-effective high throughput screening of chemical libraries for lead compounds. Identified reagents find use in the pharmaceutical industries for animal and human trials; for example, the reagents may be derivatized and rescreened in vitro and in vivo assays to optimize activity and minimize toxicity for pharmaceutical development. More specifically, neuronal cell based neural outgrowth assays, fasciculation and aggregation assays are described in detail in the experimental section below.

The invention further provides methods (also referred to herein as “screening assays”) for identifying modulators, i.e., candidate or test compounds or agents (e.g., peptides, peptidomimetics, peptoids, small molecules or other drugs) which bind to AMIGO proteins, have a stimulatory or inhibitory effect on, for example, AMIGO expression or AMIGO activity, or have a stimulatory or inhibitory effect on, for example, the expression or activity of an AMIGO substrate. Compounds thus identified can be used to modulate the activity of AMIGOs in a therapeutic protocol, to elaborate the biological function of the AMIGO, or to identify compounds that disrupt normal AMIGO interactions. The preferred AMIGOs used in this embodiment are the AMIGO, AMIGO2 and AMIGO3 of the present invention.

In one embodiment, the invention provides assays for screening candidate or test compounds which are substrates of an AMIGO protein or polypeptide or biologically active portion thereof. In another embodiment, the invention provides assays for screening candidate or test compounds which bind to or modulate the activity of an AMIGO protein or polypeptide or biologically active portion thereof.

The test compounds of the present invention can be obtained using any of the numerous approaches in combinatorial library methods known in the art, including: biological libraries; peptoid libraries [libraries of molecules having the functionalities of peptides, but with a novel, non-peptide backbone which are resistant to enzymatic degradation but which nevertheless remain bioactive] (see, e.g., Zuckermann, R. N. et al. J. Med. Chem. 1994, 37: 2678-85); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam, K. S. (1997) Anticancer Drug Des. 12:145).

In one embodiment, an assay is a cell-based assay in which a cell which expresses an AMIGO protein or biologically active portion thereof is contacted with a test compound and the ability of the test compound to modulate AMIGO activity is determined. Determining the ability of the test compound to modulate AMIGO activity can be accomplished by monitoring, for example, cell attachment or adhesion, cell growth, neurite outgrowth, fasciculation and cell chemotaxis. The cell, for example, can be of mammalian origin, e.g., a neuronal cell. In preferred embodiment, AMIGO is expressed in neuronal cells and the ability of the test compound to modulate AMIGO activity is accomplished by monitoring neurite outgrowth or alternatively, by monitoring axonal fasciculation. In another prererred embodiment AMIGO and EGFR are co-expressed, e.g. in tumour cells of neuronal or non-neuronal origin, and the amount of phosphorylation of EGFR in monitored.

Determining the ability of the AMIGO protein or a biologically active fragment thereof, to bind to or interact with an AMIGO target molecule (comprising for example AMIGO, EGFR or AMIGO ligand) can be accomplished by one of the methods described above for determining direct binding. In a preferred embodiment, determining the ability of the AMIGO protein to bind to or interact with an AMIGO target molecule can be accomplished by determining the activity of the target molecule. For example, the activity of the target molecule can be determined by detecting induction of a cellular second messenger of the target (i.e., intracellular calcium or IP3), detecting catalytic/enzymatic activity of the target molecule upon an appropriate substrate, detecting the induction of a reporter gene (comprising a target-responsive regulatory element operatively linked to a nucleic acid encoding a detectable marker, e.g., luciferase), or detecting a target-regulated cellular response (i.e., cell attachment, adhesion, growth or migration).

In yet another embodiment, an assay of the present invention is a cell-free assay in which an AMIGO protein or biologically active portion thereof is contacted with a test compound and the ability of the test compound to bind to the AMIGO protein or biologically active portion thereof is determined. Preferred biologically active portions of the AMIGO proteins to be used in assays of the present invention include fragments which participate in interactions with AMIGO, EGFR or AMIGO ligand protein. Preferably, these fragments comprise extracellular parts of the AMIGO or EGFR proteins.

The cell-free assays of the present invention are amenable to use of both soluble and/or membrane-bound forms of isolated AMIGO proteins or biologically active portions thereof. In the case of cell-free assays in which a membrane-bound form of an AMIGO protein is used it may be desirable to utilize a solubilizing agent such that the membrane-bound form of the isolated protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton™ X-100, Triton™ X-14, Thesit™, Isotridecypoly(ethylene glycol ether)n, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl=N,N-dimethyl-3-ammonio-1-propane sulfonate.

The principle of the assays used to identify compounds that bind to the AMIGO protein involves preparing a reaction mixture of the AMIGO protein and the test compound under conditions and for a time sufficient to allow the two components to interact and bind, thus forming a complex that can be removed and/or detected in the reaction mixture. These assays can be conducted in a variety of ways. For example, one method to conduct such an assay would involve anchoring AMIGO protein or the test substance onto a solid phase and detecting AMIGO protein/test compound complexes anchored on the solid phase at the end of the reaction. In one embodiment of such a method, AMIGO protein can be anchored onto a solid surface, and the test compound, (which is not anchored), can be labeled, either directly or indirectly, with detectable labels discussed herein and which are well-known to one skilled in the art.

In more than one embodiment of the above assay methods of the present invention, it may be desirable to immobilize either AMIGO, EGFR or AMIGO ligand to facilitate separation of complexed from uncomplexed forms of one or both of the proteins, as well as to accommodate automation of the assay. Binding of a test compound to an AMIGO protein, or interaction of an AMIGO protein with AMIGO, EGFR or AMIGO ligand in the presence and absence of a candidate compound, can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix. For example, glutathione-S-transferase/AMIGO fusion proteins or glutathione-S-transfera-se/target fusion proteins can be adsorbed onto glutathione sepharose beads (Sigma Chemical, St. Louis, Mo.) or glutathione derivatized microtiter plates, which are then combined with the test compound or the test compound and either the non-adsorbed AMIGO, EGFR or AMIGO ligand protein, and the mixture incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described above. Alternatively, the complexes can be dissociated from the matrix, and the level of AMIGO binding or activity determined using standard techniques.

In order to conduct the assay, the nonimmobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously nonimmobilized component is pre-labeled, the detection of label immobilized on the surface indicates that complexes were formed. Where the previously nonimmobilized component is not pre-labeled, an indirect label can be used to detect complexes anchored on the surface; e.g., using a labeled antibody specific for the immobilized component (the antibody, in turn, can be directly labeled or indirectly labeled with, e.g., a labeled anti-Ig antibody).

In one embodiment, this assay is performed utilizing antibodies reactive with AMIGO protein, EGFR or AMIGO ligand but which do not interfere with binding of the AMIGO protein to AMIGO, EGFR or AMIGO ligand. Such antibodies can be derivatized to the wells of the plate, and AMIGO, EGFR, or AMIGO ligand trapped in the wells by antibody conjugation. Methods for detecting such complexes, in addition to those described above for the GST-immobilized complexes, include immunodetection of complexes using antibodies reactive with the AMIGO, EGFR or AMIGO ligand, as well as enzyme-linked assays which rely on detecting an enzymatic activity associated with the AMIGO protein, EGFR or AMIGO ligand.

Alternatively, in another embodiment, an assay can be conducted in a liquid phase. In such an assay, the reaction products are separated from unreacted components, by any of a number of standard techniques, including but not limited to: differential centrifugation, chromatography, electrophoresis and immunoprecipitation. In differential centrifugation, complexes of molecules may be separated from uncomplexed molecules through a series of centrifugal steps, due to the different sedimentation equilibria of complexes based on their different sizes and densities (see, for example, Rivas, G., and Minton, A. P., Trends Biochem Sci 1993 August;18(8):284-7). Standard chromatographic techniques may also be utilized to separate complexed molecules from uncomplexed ones. For example, gel filtration chromatography separates molecules based on size, and through the utilization of an appropriate gel filtration resin in a column format, for example, the relatively larger complex may be separated from the relatively smaller uncomplexed components. Similarly, the relatively different charge properties of the complex as compared to the uncomplexed molecules may be exploited to differentially separate the complex from the remaining individual reactants, for example through the use of ion-exchange chromatography resins. Such resins and chromatographic techniques are well known to one skilled in the art (see, e.g., Heegaard, N. H., J Mol Recognit 1998 Winter; 11(1-6):141-8; Hage, D. S., and Tweed, S. A. J Chromatogr B Biomed Sci Appl 1997 Oct. 10;699(1-2):499-525). Gel electrophoresis may also be employed to separate complexed molecules from unbound species (see, e.g., Ausubel, F. et al., eds. Current Protocols in Molecular Biology 1999, J. Wiley: New York.). In this technique, protein or nucleic acid complexes are separated based on size or charge, for example. In order to maintain the binding interaction during the electrophoretic process, nondenaturing gels in the absence of reducing agent are typically preferred, but conditions appropriate to the particular interactants will be well known to one skilled in the art. Immunoprecipitation is another common technique utilized for the isolation of a protein-protein complex from solution (see, for example, Ausubel, F. et al., eds. Current Protocols in Molecular Biology 1999, J. Wiley: New York). In this technique, all proteins binding to an antibody specific to one of the binding molecules are precipitated from solution by conjugating the antibody to a polymer bead that may be readily collected by centrifugation. The bound proteins are released from the beads (through a specific proteolysis event or other technique well known in the art which will not disturb the protein-protein interaction in the complex), and a second immunoprecipitation step is performed, this time utilizing antibodies specific for a different interacting protein. In this manner, only the complex should remain attached to the beads. The captured complex may be visualized using gel electrophoresis. The presence of a molecular complex (which may be identified by any of these techniques) indicates that a specific binding event has occurred, and that the introduced compound specifically binds to the target protein. Further, fluorescence energy transfer may also be conveniently utilized, as described herein, to detect binding without further purification of the complex from solution.

In a preferred embodiment, the assay includes contacting the AMIGO protein or biologically active portion thereof with a known compound which binds AMIGO to form an assay mixture, contacting the assay mixture with a test compound, and determining the ability of the test compound to interact with an AMIGO protein, wherein determining the ability of the test compound to interact with an AMIGO protein comprises determining the ability of the test compound to preferentially bind to AMIGO or biologically active portion thereof as compared to the known compound. In further preferred embodiment, AMIGO protein or biologically active portion thereof is contacted with AMIGO protein and the ability of the test compound to interact with AMIGO is compared to known AMIGO:AMIGO interaction. In a still further embodiment, AMIGO protein or biologically active portion thereof is contacted with EGFR protein and the ability of the test compound to interact with AMIGO is compared to known AMIGO:EGFR interaction.

In yet another embodiment, the cell-free assay involves contacting an AMIGO protein or biologically active portion thereof with a known compound which binds the AMIGO protein to form an assay mixture, contacting the assay mixture with a test compound, and determining the ability of the test compound to interact with the AMIGO protein, wherein determining the ability of the test compound to interact with the AMIGO protein comprises determining the ability of the AMIGO protein to preferentially bind to or modulate the activity of an AMIGO, EGFR or AMIGO ligand.

The AMIGO proteins of the invention can, in vivo, interact with one or more cellular or extracellular macromolecules, such as proteins. For the purposes of this discussion, such cellular and extracellular macromolecules are referred to herein as “binding partners.” Compounds that disrupt such interactions can be useful in regulating the activity of the AMIGOs. Such compounds can include, but are not limited to molecules such as antibodies, peptides, and small molecules. The preferred proteins for use in this embodiment are the AMIGO proteins herein identified. Towards this purpose, in an alternative embodiment, the invention provides methods for determining the ability of the test compound to modulate the activity of an AMIGO protein through modulation of the activity of a downstream effector of an AMIGO, EGFR or AMIGO ligand. For example, the activity of the effector molecule on an AMIGO, EGFR or AMIGO ligand can be determined, or the binding of the effector to AMIGO, EGFR or AMIGO ligand can be determined as previously described.

The basic principle of the assay systems used to identify compounds that interfere with the interaction between the AMIGO and its cellular or extracellular binding partner or partners involves preparing a reaction mixture containing the AMIGO, and the binding partner under conditions and for a time sufficient to allow the two products to interact and bind, thus forming a complex. In order to test an agent for inhibitory activity, the reaction mixture is prepared in the presence and absence of the test compound. The test compound can be initially included in the reaction mixture, or can be added at a time subsequent to the addition of the AMIGO and its cellular or extracellular binding partner. Control reaction mixtures are incubated without the test compound or with a placebo. The formation of any complexes between the AMIGO and the cellular or extracellular binding partner is then detected. The formation of a complex in the control reaction, but not in the reaction mixture containing the test compound, indicates that the compound interferes with the interaction of the AMIGO and the interactive binding partner. Additionally, complex formation within reaction mixtures containing the test compound and AMIGO can also be compared to complex formation within reaction mixtures containing the test compound and mutant AMIGO. This comparison can be important in those cases wherein it is desirable to identify compounds that disrupt interactions of mutant but not normal AMIGOs.

The assay for compounds that interfere with the interaction of the AMIGOs and binding partners can be conducted in a heterogeneous or homogeneous format. Heterogeneous assays involve anchoring either the AMIGO or the binding partner onto a solid phase and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction between the AMIGOs and the binding partners, e.g., by competition, can be identified by conducting the reaction in the presence of the test substance; i.e., by adding the test substance to the reaction mixture prior to or simultaneously with the AMIGO and interactive cellular or extracellular binding partner. Alternatively, test compounds that disrupt preformed complexes, e.g., compounds with higher binding constants that displace one of the components from the complex, can be tested by adding the test compound to the reaction mixture after complexes have been formed. The various formats are briefly described below.

In a heterogeneous assay system, either the AMIGO or the interactive cellular or extracellular binding partner, is anchored onto a solid surface, while the non-anchored species is labeled, either directly or indirectly. In practice, microtitre plates are conveniently utilized. The anchored species can be immobilized by non-covalent or covalent attachments. Non-covalent attachment can be accomplished simply by coating the solid surface with a solution of the AMIGO or binding partner and drying. Alternatively, an immobilized antibody specific for the species to be anchored can be used to anchor the species to the solid surface. The surfaces can be prepared in advance and stored.

In order to conduct the assay, the partner of the immobilized species is exposed to the coated surface with or without the test compound. After the reaction is complete, unreacted components are removed (e.g., by washing) and any complexes formed will remain immobilized on the solid surface. The detection of complexes anchored on the solid surface can be accomplished in a number of ways. Where the non-immobilized species is pre-labeled, the detection of label immobilized on the surface indicates that complexes were formed. Where the non-immobilized species is not pre-labeled, an indirect label can be used to detect complexes anchored on the surface; e.g., using a labeled antibody specific for the initially non-immobilized species (the antibody, in turn, can be directly labeled or indirectly labeled with, e.g., a labeled anti-Ig antibody). Depending upon the order of addition of reaction components, test compounds that inhibit complex formation or that disrupt preformed complexes can be detected.

Alternatively, the reaction can be conducted in a liquid phase in the presence or absence of the test compound, the reaction products separated from unreacted components, and complexes detected; e.g., using an immobilized antibody specific for one of the binding components to anchor any complexes formed in solution, and a labeled antibody specific for the other partner to detect anchored complexes. Again, depending upon the order of addition of reactants to the liquid phase, test compounds that inhibit complex or that disrupt preformed complexes can be identified.

In an alternate embodiment of the invention, a homogeneous assay can be used. In this approach, a preformed complex of the AMIGO and the interactive cellular or extracellular binding partner product is prepared in that either the AMIGOs or their binding partners are labeled, but the signal generated by the label is quenched due to complex formation (see, e.g., U.S. Pat. No. 4,109,496 that utilizes this approach for immunoassays). The addition of a test substance that competes with and displaces one of the species from the preformed complex will result in the generation of a signal above background. In this way, test substances that disrupt AMIGO-cellular or extracellular binding partner interaction can be identified.

Assays for the Detection of the Ability of a Test Compound to Modulate Expression of AMIGO

In another embodiment, modulators of AMIGO expression are identified in a method wherein a cell is contacted with a candidate compound and the expression of AMIGO mRNA or protein in the cell is determined. The level of expression of AMIGO mRNA or protein in the presence of the candidate compound is compared to the level of expression of AMIGO mRNA or protein in the absence of the candidate compound. The candidate compound can then be identified as a modulator of AMIGO expression based on this comparison. For example, when expression of AMIGO mRNA or protein is greater (statistically significantly greater) in the presence of the candidate compound than in its absence, the candidate compound is identified as a stimulator of AMIGO mRNA or protein expression. Alternatively, when expression of AMIGO mRNA or protein is less (statistically significantly less) in the presence of the candidate compound than in its absence, the candidate compound is identified as an inhibitor of AMIGO mRNA or protein expression. The level of AMIGO mRNA or protein expression in the cells can be determined by methods described herein for detecting AMIGO mRNA or protein.

Combination Assays

In another aspect, the invention pertains to a combination of two or more of the assays described herein. For example, a modulating agent can be identified using a cell-based or a cell free assay, and the ability of the agent to modulate the activity of an AMIGO protein can be confirmed in vivo, e.g., in an animal such as an animal model for CNS disorders, or for cellular transformation and/or neuronal regeneration.

This invention farther pertains to novel agents identified by the above-described screening assays. Accordingly, it is within the scope of this invention to further use an agent identified as described herein in an appropriate animal model. For example, an agent identified as described herein (e.g., an AMIGO modulating agent, an antisense AMIGO nucleic acid molecule, an AMIGO-specific antibody, or an AMIGO-binding partner) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

The choice of assay format will be based primarily on the nature and type of sensitivity/resistance protein being assayed. A skilled artisan can readily adapt protein activity assays for use in the present invention with the genes identified herein.

Diagnostics

The invention also features diagnostic or prognostic kits for use in detecting the presence of AMIGO or allelic variant thereof in a biological sample. The kit provides means for the diagnostics of AMIGO dependent conditions as described hereinabove or for assessing the predisposition of an individual to conditions mediated by variation or dysfunction of AMIGO. The kit can comprise a labeled compound capable of detecting AMIGO polypeptide or nucleic acid (e.g. mRNA) in a biological sample. The kit can also comprise nucleic acid primers or probes capable of hybridising specifically to at least of portion of an AMIGO gene or allelic variant thereof. The kit can be packaged in a suitable container and preferably it contains instructions for using the kit.

Purification of AMIGO Binding Molecules

In yet another aspect of the invention, the AMIGO or AMIGO analog may be used for affinity purification of molecules (receptors) that bind to the AMIGO. AMIGO is a preferred ligand for purification. Briefly, this technique involves: (a) contacting a source of AMIGO receptor with an immobilized AMIGO under conditions whereby the AMIGO receptor to be purified is selectively adsorbed onto the immobilized AMIGO; (b) washing the immobilized AMIGO and its support to remove non-adsorbed material; and (c) eluting the AMIGO receptor molecules from the immobilized AMIGO to which they are adsorbed with an elution buffer. In a particularly preferred embodiment of affinity purification, AMIGO is covalently attaching to an inert and porous matrix or resin (e.g., agarose reacted with cyanogen bromide). Especially preferred is an AMIGO immunoadhesin immobilized on a protein-A column. A solution containing AMIGO receptor is then passed through the chromatographic material. The AMIGO receptor adsorbs to the column and is subsequently released by changing the elution conditions (e.g. by changing pH or ionic strength).

The preferred technique for identifying molecules which bind to the AMIGO utilizes a chimeric AMIGO (e.g., epitope-tagged AMIGO or AMIGO immunoadhesin) attached to a solid phase, such as the well of an assay plate. The binding of the candidate molecules, which are optionally labelled (e.g., radiolabeled), to the immobilized AMIGO can be measured.

Production of Transgenic Animals

Nucleic acids which encode AMIGO, preferably from non-human species, such as murine or rat protein, can be used to generate either transgenic animals or “knock out” animals which, in turn, are useful in the development and screening of therapeutically useful reagents. A transgenic animal (e.g., a mouse) is an animal having cells that contain a transgene, which transgene was introduced into the animal or an ancestor of the animal at a prenatal, e.g., an embryonic, stage. A transgene is a DNA which is integrated into the genome of a cell from which a transgenic animal develops. In one embodiment, the human and/or mouse cDNA encoding AMIGO, or an appropriate sequence thereof, can be used to clone genomic DNA encoding AMIGO in accordance with established techniques and the genomic sequences used to generate transgenic animals that contain cells which express DNA encoding AMIGO. Methods for generating transgenic animals, particularly animals such as mice, have become conventional in the art and are described, for example, in U.S. Pat. Nos. 4,736,866 and 4,870,009. Typically, particular cells would be targeted for AMIGO transgene incorporation with tissue-specific enhancers, which could result in desired effect of treatment. Transgenic animals that include a copy of a transgene encoding AMIGO introduced into the germ line of the animal at an embryonic stage can be used to examine the effect of increased expression of DNA encoding AMIGO. Such animals can be used as tester animals for reagents thought to confer protection from, for example, diseases related to AMIGO. In accordance with this facet of the invention, an animal is treated with the reagent and a reduced incidence of the disease, compared to untreated animals bearing the transgene, would indicate a potential therapeutic intervention for the disease.

It is now well-established that transgenes are expressed more efficiently if they contain introns at the 5′ end, and if these are the naturally occurring introns (Brinster et al. Proc. Natl. Acad. Sci. USA 85:836-840 (1988); Yokode et al., Science 250:1273-1275 (1990)).

Transgenic offspring are identified by demonstrating incorporation of the microinjected transgene into their genomes, preferably by preparing DNA from short sections of tail and analyzing by Southern blotting for presence of the transgene (“Tail Blots”). A preferred probe is a segment of a transgene fusion construct that is uniquely present in the transgene and not in the mouse genome. Alternatively, substitution of a natural sequence of codons in the transgene with a different sequence that still encodes the same peptide yields a unique region identifiable in DNA and RNA analysis. Transgenic “founder” mice identified in this fashion are bred with normal mice to yield heterozygotes, which are backcrossed to create a line of transgenic mice. Tail blots of each mouse from each generation are examined until the strain is established and homozygous. Each successfully created founder mouse and its strain vary from other strains in the location and copy number of transgenes inserted into the mouse genome, and hence have widely varying levels of transgene expression. Selected animals from each established line are sacrificed at 2 months of age and the expression of the transgene is analyzed by Northern blotting of RNA from liver, muscle, fat, kidney, brain, lung, heart, spleen, gonad, adrenal and intestine.

Production of “Knock Out” Animals

Alternatively, the non-human homologs of AMIGO can be used to construct an AMIGO “knock out” animal, i.e., having a defective or altered gene encoding AMIGO, as a result of homologous recombination between the endogenous AMIGO gene and an altered genomic AMIGO DNA introduced into an embryonic cell of the animal. For example, murine AMIGO cDNA can be used to clone genomic AMIGO DNA in accordance with established techniques. A portion of the genomic AMIGO DNA can be deleted or replaced with another gene, such as a gene encoding a selectable marker which can be used to monitor integration. Typically, several kilobases of unaltered flanking DNA (both at the 5′ and 3′ ends) are included in the vector (see e.g., Thomas and Capecchi, Cell 51:503 (1987) for a description of homologous recombination vectors). The vector is introduced into an embryonic stem cell line (e.g., by electroporation) and cells in which the introduced DNA has homologously recombined with the endogenous DNA are selected (see e.g., Li et al., Cell 69:915 (1992)). The selected cells are then injected into a blastocyst of an animal (e.g., a mouse) to form aggregation chimeras (see e.g., Bradley, in Teratocarcinomas and Embryonic Stem Cells: A Practical Approach, E. J. Robertson, ed. (IRL, Oxford, 1987), pp. 113-152). A chimeric embryo can then be implanted into a suitable pseudopregnant female foster animal and the embryo brought to term to create a “knock out” animal. Progeny harbouring the homologously recombined DNA in their germ cells can be identified by standard techniques and used to breed animals in which all cells of the animal contain the homologously recombined DNA. Knockout animals can be characterized for their ability to mimic human neurological disorders and defects.

Equivalents

Although particular embodiments have been disclosed herein in detail, this has been done by way of example for purposes of illustration only, and is not intended to be limiting with respect to the scope of the appended claims that follow. In particular, it is contemplated by the inventors that various substitutions, alterations, and modifications may be made to the invention without departing from the spirit and scope of the invention as defined by the claims. The choice of nucleic acid starting material, clone of interest, or library type is believed to be a matter of routine for a person of ordinary skill in the art with knowledge of the embodiments described herein. Other aspects, advantages, and modifications considered to be within the scope of the following claims.

Experimental Section

Materials and Methods

Ordered Differential Display

Ordered Differential Display was performed as described by Matz et al. (1997) comparing genes induced on amphoterin versus laminin matrix. Hippocampi were dissected from 18-d-old rat embryos and triturated with pasteur pipette in Hank's balanced salt solution (HBSS w/o Ca & Mg, GIBCO BRL) containing 1 mM sodium pyruvate and 10 mM Hepes, pH 7.4. After washing in HBSS, neurons were suspended in Neurobasal medium (GIBCO BRL), 2% B27 supplement (GIBCO BRL), 25 μM L-glutamic acid (Sigma-Aldrich), and 1% L-glutamine (GIBCO BRL) and they were then seeded at the density of 10⁶ cells on 35 mm plastic plates (Greiner) coated with laminin (10 μg/ml; Sigma-Aldrich) or recombinant amphoterin (10 μg/ml). RNA was isolated by using RNeasy mini kit (Qiagen) 24 hours after seeding and was used for ordered differential display.

Cloning of the AMIGO, AMIGO2 and AMIGO3 cDNAs

The rat AMIGO cDNA 5′ end was amplificated by using method of Matz et al. (1999) based on template-switching effect and step-out PCR, and the full-length cDNA was cloned from postnatal day 14 rat cerebrum using RT-reaction with the following primers: 5′ primer ACTGCTTCTCGCCTGGCCCGT (SEQ ID NO: 42); and 3′ primer GAACCTCCCCATCAGCCTATACTG (SEQ ID NO: 43). The rat AMIGO sequence was used to find out human and mouse ESTs to get sequences for cloning of the human and mouse AMIGOs. The human AMIGO cDNA was cloned from the THP-1 cell-line (ATCC #TIB-202) using an RT-reaction with the following primers: 5′ primer CAGAACATGCCCGGGTGAC (SEQ ID NO: 44); and 3′ primer GGACCAATTCCCTTGAGGTCAG (SEQ ID NO: 45). The mouse AMIGO cDNA was cloned from adult mouse cerebrum using an RT-reaction with the following primers: 5′ primer ACTGCTTCTCGCCTGGCCCGT (SEQ ID NO: 46); and 3′ primer AACCTCCCCATCAGCCTACGCTG (SEQ ID NO: 47). The AMIGO sequences were used for homology search with BLAST to find possible other related sequences. The human AMIGO2 cDNA was cloned from the HT1080 cell line (ATCC #CCL-121) as above: the 5′ primer was CTCAGAGGCGACCATAATGTC (SEQ ID NO: 48) and the 3′ primer was TGTTTATTTTGCAGACCACACAC (SEQ ID NO: 49). The mouse AMIGO2 cDNA was cloned from adult mouse cerebrum with the following primers: 5′ primer CTCAGAGGCGACCATAATGTC (SEQ ID NO: 50); and 3′ primer GCGATGCTGAAGGCTAAGATG (SEQ ID NO: 51). The human AMIGO3 cDNA was cloned from the HEK293 cell line (ATCC #CRL-1573) with the 5′ primer CAACCTGCACAGAGCTGCTCTGTAC (SEQ ID NO: 52) and the 3′ primer GCACAGTGCTTCCCACCAGTATCTG (SEQ ID NO: 53). The mouse AMIGO3 cDNA was cloned from adult mouse cerebellum with the 5′ primer AGAAGTAGGTGAGTCTTGGAGCT (SEQ ID NO: 54) and the 3′ primer TGTTGTGCAGGTAGAGCCTG (SEQ ID NO: 55).

RT-PCR and In Situ Hybridization

Total RNA was reverse transcribed in a reaction containing 1 μg RNA, 0.25 mM dNTP-mix, 1 μg random nonamers, 20 U recombinant Rnasin (Promega), 200 U MMLV-RT (Promega) with 1×MMLV reaction buffer supplied. 2 μl of the reverse transcription mixture was then used for polymerase chain reaction with gene specific primers. For the mouse AMIGO the primers where as follows: 5′ primer AGCAACATCCTCAGCTGCTC (SEQ ID NO: 56); and 3′ primer CTTCAGCTTGTTGGAGGACAG (SEQ ID NO: 57). For mouse AMIGO2 the primers were: 5′ primer GGCACTTTAGCTCCGTGATG (SEQ ID NO: 58); and 3′ primer GTCTCGTTTAACAGCCGCTG (SEQ ID NO: 59). For the mouse AMIGO3 the primers were: 5′ primer AGGTGTCAGAGTCCCGAGTG (SEQ ID NO: 60); and 3′ primer GTAGAGCAACACCAGCACCA (SEQ ID NO: 61). For GAPDH control the primers were: 5′ primer CAACGACCCCTTCATTGACC (SEQ ID NO: 62); and 3′ primer AGTGATGGCATGGACTGTGG (SEQ ID NO: 63).

The subsequent PCR reaction was performed in a PCR mix (2.5 μM dNTP, 10 mM Tris-HCL, pH 8.8, 150 mM KCL, 1.5 mM MgCl₂, 0.1% Triton X-100) containing 0.2 μM 5′ primer and 3′ primer and 1 unit of DYNAzyme II DNA Polymerase (Finnzymes). The amplification products were separated on 1.5% agarose gel and stained with EtBr.

For in situ hybridization with radiolabeled probes, a 1.2-kb fragment from the mouse AMIGO cDNA was PCR amplified with the following primers: 5′ primer CCGCTCGAGCCGGCCGATCTGTGGTFAG (SEQ ID NO: 64); and 3′ primer CGGAATTCTCACACCACAATGGGTCTATCAGA (SEQ ID NO: 65). The reaction product was then ligated into pGEM-T vector. In situ hybridization analysis was carried out using single-stranded RNA probes on mouse fetal and adult paraffin embedded tissue sections as described previously (Reponen et al., 1994).

Production of AMIGO Ig-Fusion Protein

A 1180-bp BamHI fragment containing the entire extracellular coding region of the mouse AMIGO was amplified by PCR with the following primers: 5′ primer CGGGATCCTAGGGTGACTCTCTCCCAGATCC (SEQ ID NO: 66); and 3′ primer CGGGATCCGTTGAGGGTGTCATGGTGTCC (SEQ ID NO: 67). The reaction product was then ligated into pRMHA3-3c-FC-cDNA. The AMIGO Ig-fusion protein plasmid was cotransfected with the hygromycin resistance plasmid p-COP-hyg into Drosophila S2-cells by using the Fugene6 transfection reagent (ROCHE). After a three weeks selection with 300 μg/ml hygromycin B (Calbiochem), stabile AMIGO Ig-fusion S2-cell pools were cultured in shake flasks where the protein expression was induced with 500 μM CuSO₄. After culturing for 6 days the AMIGO Ig-fusion protein was isolated from the supernatant by using protein-A agarose (Upstate) according to the manufacturer's instructions.

Antibodies, Western Blotting and Immunohistochemistry

Rabbit anti-AMIGO peptide antibodies were raised against the synthetic peptide YAMGETFNET (SEQ ID NO: 68) (corresponding to amino acids 341-350 of the mouse AMIGO and 342-351 of the rat and human AMIGO). Binding of the antibodies to AMIGO was verified using the recombinant AMIGO Ig-fusion protein and crude brain extracts in Western blotting (see below). Since the antibodies bound more intensely and specifically to the rat AMIGO compared to AMIGO from other species (possibly due to species differences in the glycosylation site close to the peptide sequence used in immunization), rat samples were primarily used in immunochemical detections.

Brains of embryonic, postnatal and adult rats were extracted to the final concentration of 83.3 mg tissue/ml SDS-extraction buffer (62,5 mM Tris, 1,8% SDS, 7,75% glycerol, 4,4% 2-mercaptoethanol, pH 6.8). After addition of the SDS buffer, the extracts were pressed several times through a needle. The extracts were boiled 2×5 min and centrifuged at 10 000×g for 10 min to remove nonsoluble material. Samples corresponding to the same wet weight of tissue were analysed by Western blotting. Ponceau staining of the membrane confirmed uniform protein amounts.

Precast 4-15% gels (Bio-Rad) were used for SDS-PAGE in Western blotting. Proteins were transferred to Hybond™ nitrocellulose membrane (Amersham Pharmacia Biotech) by Semi-dry blotting technique. Rabbit anti-AMIGO peptide antibody (1/1000 dilution) and monoclonal anti-CNPase, clone 11-5B (Sigma, 1/1500) were used as primary antibodies. HRP-conjugated goat anti-rabbit IgG (Bio-Rad) and sheep anti-mouse IgG (AP Biotech) were used as secondary antibodies. The antibody comples were detected using ECL™ reagents (AP Biotech).

Immunohistochemistry of AMIGO was performed using paraffine sections. In brief, adult rats were sacrificed after CO₂ treatment by cervical dislocation and tissues were fixed by using ice-cold PBS with 4% paraformaldehyde, and the samples were then transferred in paraffine. Hydrated paraffine sections (4-10 μm thick) were incubated with 1% hydrogen peroxide/methanol solution for 20 min, and washed again with PBS. The sections were blocked for 1 h with 5% skimmed milk powder in PBS. The sections were then incubated with the rabbit AMIGO peptide antiserum, which was diluted 1/200 in the blocking buffer at +4° C. overnight. After washing with PBS, the sections were incubated with HRP conjugated goat anti-rabbit antibodies (Biorad) at a dilution of 1:500 for 2 h at room temperature, washed with PBS and incubated with aminoethyl carbazole (AEC, Sigma) as a chromogenic substrate. Immunofluorescence staining for in vitro cultured hippocampal neurons was performed by using FITC conjucated goat anti-rabbit secondary antibodies (Jackson lab).

Neurite Outgrowth Assay

Hippocampi were dissected from 18-day-old rat embryos into a Ca—Mg-free trituration medium (HBSS with 1 mM sodium pyruvate and 10 mM HEPES, pH 7.4). Cells were dissociated by pipetting 25 times with glass pasteur pipette and washed once with the Ca—Mg-containing buffer (HBSS+Ca+Mg with 1 mM sodium pyruvate and 10 mM HEPES, pH 7.4). The cells were seeded at the density of 70000 cells/cm² on 96-well polystyrene dishes coated by the test protein in Neurobasal medium with 2% B27 supplement (GIBCO BRL), 1% BSA, 0.5 mM L-glutamine, 25 μM L-glutamic acid and 1× penicillin-streptomycin. The dishes were coated with the test protein (3.125-100 μg/ml) in PBS overnight at 4° C., washed tree times with PBS, and blocked with 1% BSA in PBS for 1 h at room temperature before adding the cells. The cells were cultured for 24 h before counting the neurite outgrowth. For counting of neurite outgrowth, images were taken from living cells using randomly selected microscopic fields and the extensions, which were twice the length of the cell soma, were considered as neurites. For quantification of neurite outgrowth, 15 images (275 μm×225 μm) with a total of 750 cells were evaluated from every concentration of the test protein (AMIGO Ig-fusion or Fc control substrate) used for coating. The data were pooled from three independent experiments.

To test the effect of soluble AMIGO Ig-fusion protein the dishes were coated with the AMIGO Ig-fusion protein (12.5 μg/ml in PBS) at 4° C. overnight, washed three times with PBS, and blocked with 1% BSA in PBS for 1 h at room temperature. The cells were seeded at the density of 70000 cells/cm² and cultured for 24 h before counting the neurite outgrowth. Counting was carried out as above from three independent experiments. A total of 750 cells were evaluated for every concentration of the test protein (AMIGO Ig-fusion or the Fc control protein) used in solution.

In Vitro Fasciculation Assay

Fasciculation of neurites was studied with hippocampal neurons prepared as above. The 96-well plates were coated with poly-L-lysine at +4° C. overnight, washed three times with PBS, and blocked with 1% BSA in PBS for 1 h at room temperature. The cells were seeded at the density of 70000 cells/cm² in the serum free medium (see “Neurite outgrowth assay”) with either the AMIGO Ig-fusion protein or the Fc control protein in solution. The AMIGO Ig-fusion and the Fc control protein were tested at 3.25-25 μg/ml. The experiment was repeated independently 3 times, and pictures were taken from living cells after 4 days in culture. For quantification of neurite outgrowth, 12 randomly taken images (45 μm×35 μm) were taken for every concentration of the AMIGO Ig-fusion and the Fc control protein used in solution. To evaluate inhibition of fasciculation, the total length of the processes, the diameter of which is <2 μm (formed only from 1-3 neurites), was measured from the 12 images taken for every protein concentration tested.

Pictures for the neurite outgrowth and fasciculation experiments were taken with Olympus DP10 digital camera. The measurements were carried out by using the Image-Pro image analysis software.

Binding Assays

Coimmunoprecipitation experiments were performed using transiently transfected HEK293T cells. The constructs were transfected into the cells by using FUGENE6 (ROCHE) according to the manufacturer's instructions. The full length AMIGO was cloned in frame with the pEGFP-N1 (Clontech) and pcDNA6-V5-His (Invitrogen) vectors. The full length RAGE was cloned in frame with the pcDNA6-V5-His vector. After transfection, the cells were grown for 48 h before lysing in the RIPA buffer with 10 mg/ml PMSF and 60 μg/ml aprotinin (SIGMA). Coimmunoprecipitation experiments were carried out using rabbit anti-GFP antibody (Santa Cruz; sc-8334) and mouse anti-V5 antibody (Invitrogen; 46-0705) at the concentration of 1 μg/ml.

The aggregation assay was carried out using protein A Fluoresbrite carboxylated beads (Polysciences, size 1 μm). The beads (100 μg) were first washed 3 times with PBS, 2% BSA, 0.1% Tween-20 solution and they were the mixed and sonicated in water bath in 50 μl of the buffer mentioned above. The beads were divided to two aliquots, and the test and the control protein (10 μg each) were added into the beads in 25 μl of PBS, 2% BSA and 0.1% Tween-20 solution (final volume 50 μl). After addition of the protein 2 μl samples were taken into 100 μl of PBS, 2% BSA, 0.1% Tween-20 solution in 96-well plate at different time points. The plate was incubated at room temperature and the aggregation was evaluated under the fluorescence microscope. Kinetics of bead aggregation was calculated from three independent experiments from 12 fields containing 1500 beads. The extent of bead aggregation is represented by the index Nₜ/N₀ where Nₜ and N₀ are the total number of particles at the incubation times t and 0 (Agarwala et al., 2001).

Coimmunoprecipitation of AMIGO and AMIGO2 with EGFR

Coimmunoprecipitation experiments were performed using stable HEK293 cells expressing EGFR. The constructs were transfected into the cells by using FUGENE6 (ROCHE) according to the manufacturer's instructions. The full length and extracellular part (EC-part) AMIGO, AMIGO2 and AMIGO3 were cloned in frame pcDNA6-V5-His (Invitrogen) vectors. After transfection, the cells were grown for 48 h before lysing in the RIPA buffer with 10 mg/ml PMSF, 60 μg/ml aprotinin (SIGMA) and 1 mM EDTA. Coimmunoprecipitation experiments were carried out using rabbit anti-EGFR antibody (Santa Cruz) and mouse anti-V5 antibody (Invitrogen; 46-0705) at the concentration of 1 μg/ml.

EGFR Phosphorylation Experiment

EGFR phoshorylation experiments were performed using HEK293T cells. The constructs were transfected into the cells by using FUGENE6 (ROCHE) according to the manufacturer's instructions. The full length AMIGO, AMIGO2 and AMIGO3 were cloned in frame with pcDNA6-V5-His vector (Invitrogen). The full length human EGFR was cloned with C-terminal Flag-tag into pcDNA6 vector (Invitrogen). The cells on 50% confluent 6 cm plate were transfected with 0.3 μg of EGFR plasmid and with 1.7 μg of AMIGO, AMIGO2, AMIGO3 or control plasmid (pcDNA6-V5-His, Invitrogen). After 24 hours of transfection cells were starved for 4 hours without serum. The autophosphorylation of the EGFR was induced by adding 50 ng/ml of EGF for 5 minutes in +37° C. The cells were lysed and immunoprecipitated with anti-phospho-Tyrosine antibody (clone PY20). The cells were also immunoprecipitated with anti-Flag-tag antibody (clone M2). The samples from anti-phospho-Tyrosine immunoprecipitation were detected on western blot by using the anti-flag-tag antibody to see the EGFR phosphorylation differences between the samples. The samples from anti-Flag-tag immunoprecipitation were detected on western blot by using the anti-phospho-Tyrosine antibody to see the EGFR phosphorylation differences between the samples.

Homo- and Heterophilic Binding of AMIGO, AMIGO2 and AMIGO3

Coimmunoprecipitation experiments were performed using transiently transfected HEK293T cells. The constructs were transfected into the cells by using FUGENE6 (ROCHE) according to the manufacturer's instructions. The full length and extracellular part (EC-part) AMIGO, AMIGO2 and AMIGO3 were cloned in frame with the pEGFP-N1 (Clontech) or pcDNA6-V5-His (Invitrogen) vectors. The full length RAGE was cloned in frame with the pcDNA6-V5-His vector. After transfection, the cells were grown for 48 h before lysing in the RIPA buffer with 10 mg/ml PMSF and 60 μg/ml aprotinin (SIGMA).

Coimmunoprecipitation experiments were carried out using rabbit anti-GFP antibody (Santa Cruz; sc-8334) and mouse anti-V5 antibody (Invitrogen; 46-0705) at the concentration of 1 μg/ml.

Knockout Constructs for AMIGO, AMIGO2 and AMIGO3.

For AMIGO gene targeting, we constructed a replacement vector by using genomic DNA fracments from mouse phage library (strain 129SV). The whole coding region of AMIGO gene was replaced by inserting Beta-galactosidase gene under the promoter of AMIGO gene using tailored PCR primers: 5′ primer GCGGCCGCTCAGGGCCCACGGTTTCTGCAG (SEQ ID NO: 69) (with NotI site) and 3′ primer GGCGCGCCACTGGGAAGAGVGAGGAAGGCCAC (SEQ ID NO: 70) (with AscI site). For positive selection, we cloned the neomycin-resistance gene after the beta-galactosidase gene. The 3′ prime homologous arm was inserted into the vector as a KpnI/NcoI fragment (NcoI blunted). The length of the homologous recombination arms where 9.9 kb for 5′ arm and 2.0 kb for 3′ arm.

For AMIGO2 gene targeting, we constructed a replacement vector by using genomic DNA fracments from mouse phage library (strain 129SV). The whole coding region of AMIGO2 gene was replaced by inserting human placental alkaline phosphatase gene under the promoter of AMIGO2 gene using tailored PCR primers: 5′ primer TAAACTAGCGGCCGCTCATGGAGGCTCACCCATGGAC (SEQ ID NO: 71) (with NotI site) and 3′ primer AGATATGGCGCGCCGGTCGCCTCTGAGTCTCTTGCCAG (SEQ ID NO: 72) (with AscI site). For positive selection, we cloned the neomycin-resistance gene after the human placental alkaline phosphatase gene. The 3′ homologous arm was inserted into the vector as a BamHI/HindIII fragment (HindIII blunted). The length of the homologous recombination arms where 3.0 kb for 5′ arm and 3.0 kb for 3′ arm.

For AMIGO3 gene targeting, we constructed a replacement vector by using genomic DNA fracments from mouse phage library (strain 129SV). The whole coding region of AMIGO3 gene was replaced by inserting EGFP gene under the promoter of AMIGO3 gene using tailored PCR primers: 5′ primer ACCTTAATTAACCAGATGGCTTCTTCTTTC (SEQ ID NO: 73) (with PacI site) and 3′ primer AGATATGGCGCGCCAGTGACTACCAGGGAAGAT (SEQ ID NO: 74) (with AscI site). For positive selection, we cloned the neomycin-resistance gene after the EGFP gene. The 3′ homologous arm was inserted into the vector as a BamHI fragment. The length of the homologous recombination arms where 3.5 kb for 5′ arm and 2.6 kb for 3′ arm.

Using standard procedures, we electroporated R1 mouse embryonic stem cells, suspenced in PBS, with 20 μg linearized (AMIGO:NotI, AMIGO2:NotI and AMIGO3: PacI) targeting vector, using BioRad Gene Pulser (240 V and 500° F.). Transfected cells were selected with 300 μg/ml G418 (Gibco). On day 9-11 after electroporation, we picked 100-400 clones and identified resistant clones with homologous recombination by PCR amplification using primers for neomycin resistance gene and outside the targeted locus. PCR results were confirmed by using southern blots with probes outside the targeting locus.

Using standard procedures, selected embryonic stem cells were aggregated into ICR morulas and aggregates were transferred to pseudopregnant foster mothers. Highly chimeric males were bred to ICR females and heterozycos offsprings were intercrossed to obtain homozygous mutant mice. For genotyping the genomic DNA was isolated from tail biopsies with protein K digestion and isopropanol precipitation. For routing genotyping, we used PCR where first reaction contains oligos which could amplificate product only from intact AMIGO, AMIGO2 or AMIGO3 gene locus (from inside the genes). The second PCR reaction contains oligos which could only amplificate product from targeted locus (one oligo from neomycin gene and the second from 3′ homologous arm used for targeting).

These AMIGO, AMIGO2 and AMIGO3 single knockout mice strains have been used to generate double knockout mice strains (ΔAMIGO/ΔAMIGO2; ΔAMIGO/ΔAMIGO3; ΔAMIGO2/ΔAMIGO3) and triple knockout mouse strain (ΔAMIGO/ΔAMIGO2/ΔAMIGO3) by using standard breeding procedures. The genotype of the mutant mice were confirmed by using same PCR reactions as in single knockout strains.

AMIGO ig-Fusion Transgenic Animals

The DNA region encoding mouse AMIGO extracellular part was amplified by PCR from mouse AMIGO cDNA using the BamHI-containing upstream primer CGGGATCCTAGGGTGACTCTCTCCCAGATCC (SEQ ID NO: 75) and the BamHI-containing downstream primer CGGGATCCGTTGAGGGTGTCATGGTGTCC (SEQ ID NO: 76). PCR fragment was cloned into frame with human IgG FC-part in expression vector pRMHA3-3c-FC. The DNA region encoding mouse AMIGO extracellular part fused with IgG FC-part was amplified by PCR using the NotI-containing upstream primer ATAAGAATGCGGCCGCCAATGTGCATCAGTTGTGGTCAG (SEQ ID NO: 77) and the XbaI-containing downstream primer GCTCTAGACGTGCCAAGCATCCTCGTGCGAC (SEQ ID NO: 78). The PCR fragment was cloned into a vector psisGI. In the resulting plasmid, the open reading frame of AMIGO ig-fusion was located under the control of a PDGF-beta promoter and supplied with the polyadenylation signal of the bovine growth hormone. The construct was injected into the pronuclei of oocytes from superovulated females of C57BL/6 strain. The transgene integration was determined by Southern blot and PCR analyses of tail DNA. To establish the transgenic line, founders were crossed with C57BL/6 animals.

Regeneration Experiment with AMIGO, AMIGO2 or AMIGO3 Proteins.

Spinal cord injury and delivery of AMIGO, AMIGO2 or AMIGO3 can be made as follows. BALB-c female mice (n=70) are anesthetized with 0.4 ml/kg hypnorm and 5 mg/kg diazepam. A segment of the thoracic spinal cord is exposed using fine rongeurs to remove the bone, and a dorsal over-hemisection was made at T7. Fine scissors are used to cut the dorsal part of the spinal cord, which is cut a second time with a fine knife to ensure that the lesion extends past the central canal. The SABER Delivery System (DURECT Corporation) is according to manufacturer's instructions AMIGO, AMIGO2 or AMIGO3 Ig-fusion proteins are added into the SABER solution in concentration of 1-100 mg/ml. As controls, a second group of animals receives SABRE solution with PBS buffer, and a third group is left untreated. For retransections 3 weeks after SCI, the spinal cords are cut at T6 as described above, and the animals are tested using the Basso-Beattie-Bresnahan (BBB) locomotor rating scale on days 1, 2, and 6 after the second surgery. Alternatively, Ig-fusion protein is replaced with AMIGO ectodomain as described below.

Axonal Regeneration Experiment with Soluble AMIGO, AMIGO2 or AMIGO3 Ectodomains.

Spinal cord dorsal hemisection and corticospinal fiber tracing is adapted from GrandPre et al. (2002) Nogo-66 receptor antagonist peptide promotes axonal regeneration. Nature 417: 547-551. Adult female C57BL/6 mice (8-10 weeks of age) are deeply anesthetized with intramuscular ketamine (100 mg/kg) and intraperitoneal xylazine (15 mg/kg). A complete laminectomy is performed, and the dorsal part of spinal cord is fully exposed at levels T6 and T7. The dorsal half of the spinal cord is cut with a pair of microscissors to sever the dorsal parts of the corticospinal tracts, and the depth of lesion (approximately 1.0 mm) is assured by passing the sharp part of a number 11 blade across the dorsal half of the cord. An osmotic minipump (Alzet model 2002, Alza, Mountain View, Calif.) is implanted after the hemisection of dorsal spinal cord and positioned to deliver reagents to the subcutaneous space. A catheter connected to the outlet of the minipump is inserted into the intrathecal space of the spinal cord at the T7 level through a small hole in the dura. The pump is filled with vehicle (97.5% PBS plus 2.5% DMSO) or soluble AMIGO, AMIGO2 and/or AMIGO3 ectodomain in the vehicle. The vehicle or soluble AMIGO, AMIGO2 and/or AMIGO3 are delivered continuously at a rate of approx 0.6 μl/hr for 14 d and the soluble AMIGO, AMIGO2 and/or AMIGO3 ectodomain doses are 2.0, 7.5 and 15.0 mg·kg−1·d−1. For those mice receiving soluble AMIGO, AMIGO2 and/or AMIGO3 ectodomain without spinal cord injury, the laminectomy and minipump placement are accomplished in the same fashion. Two weeks after lesion, a burr hole is made on each side of the skull overlying the sensorimotor cortex of the lower limbs. The anterograde neuronal tracer biotin dextran amine (BDA, 10% in PBS) is applied at four injection sites at a depth of 0.5-0.8 mm from the cortical surface on each side. Two weeks after BDA injection, the animals are killed by perfusion with PBS, followed by 4% paraformadehyde. The spinal cord extending from 6 mm rostral to 6 mm caudal from the lesion site is cut parasaggitally (50 μm) on a vibrating microtome. Transverse sections are collected from the spinal cord 8-12 mm rostral to and 8-12 mm caudal to the injury site. The sections are incubated with avidin-biotin-peroxidase complex and the BDA tracer for regenerated axons is visualized by nickel-enhanced diaminobenzidine HRP reaction. For bevioral analysis vehicle-treated and soluble AMIGO, AMIGO2 or AMIGO3 ectodomain treated mice are compared using the Basso-Beattie-Bresnahan (BBB) locomotor rating scale according to Basso et al (1995) A sensitive and reliable locomotor rating scale for open field testing in rats. J. Neurotrauma 12, 1-21.

Inhibition of Glial Scar Formation in CNS with Soluble AMIGO, AMIGO2 and AMIGO3 Proteins

Stereotactic lesioning of the cerebral cortex and intraventricular cannulation can be made according to (Logan et al., 1994). Adult female 200- to 250-g Wistar rats are assigned to two treatment groups of 5 animals each receiving: (i) 30 μg/10 μl/day Fc-control protein; or (ii) 30 μg/10 μl/day AMIGO, AMIGO2 and/or AMIGO3 Ig-fusion protein in saline. On day 0 of the experiment, a stereotactically defined unilateral incisional lesion is placed through the cerebral cortex into the lateral ventricle at the same time as ipsilateral placement of a permanent intraventricular cannula. Reagents (10 μl) are perfused into the lesion site by daily intraventricular injections through the cannulae for 14 days under halothane anaesthesia. After 14 days post lesion (dpl), animals are killed and their brains processed for immunohistochemical analysis of the lesion site. Alternatively, Fc-fusion protein is replaced with AMIGO ectodomain in order to avoid immune response against Fc domain, hence the treatment groups of 5 animals are comprise: (i) 10 μl/day phosphate buffered saline (PBS); or (ii) 30 μg/10 μl/day soluble AMIGO, AMIGO2 or AMIGO3 ectodomain in phosphate buffered saline.

Modulation of Tumour Metastasis by Using Soluble AMIGO, AMIGO2 or AMIGO3 Extracellular Domain.

The modulation of tumour metastases assay can be performed as follows. Lewis lung murine carcinoma cells are injected into the dorsal midline of male, 6-8-week-old C57BL/6J mice (Jackson Laboratories, Bar Harbor, Me.). Primary tumours are surgically excised when tumour volume is 1,500 mm³ (day 14). For three days before the removal of primary tumor, mice receive AMIGO-, AMIGO2- or AMIGO3 Ig-fusion protein or control FC-part protein once daily, 21 days after removal of primary tumour. Weight of the lungs and numbers of lung surface metastases are determined under ×4 magnification using an Olympus microscope after intratracheal injection of India Ink (15%). Alternatively, animal experiments are adapted from Liao et al. (2000). For the pulmonary metastasis model, C57BL/6J mice (Jackson Laboratories, Bar Harbor, Me.) are injected intra-footpad with 1×10⁵ cells of murine Lewis lung carcinoma. When footpad tumors reach 5 mm in diameter, the tumor-bearing leg is surgically ligated. Mice are then divided into two groups receiving injections of approx 20-30 mg/kg/dose of either vehicle (phosphate buffered saline) or vehicle with soluble AMIGO, AMIGO2 or AMIGO3 ectodomain every 3 days for 3 weeks. Weight of the lungs and numbers of lung surface metastases are determined under ×4 magnification using an Olympus microscope after intratracheal injection of India Ink (15%).

Blockage of Local Tumour Growth with Soluble AMIGO, AMIGO2 or AMIGO3 Extracellular Domain.

Rat C6 glioma cells are injected into the dorsal midline of female NCR immunocompromised mice aged 4-6 weeks (Taconic Farms, Germantown, N.Y.). Alternatively, rat C6 glioma cells are injected into the dorsal midline of female mice with severe combined immunodeficiency (SCID; Taconic Farms). Administration of AMIGO-, AMIGO2- or AMIGO3 Ig-fusion protein or control FC-part protein is done once daily to immunocompromised (athymic nude) mice upon injection of rat C6 glioma cells. Tumours are measured at day 21 with calipers and the volume is calculates: V=π*h(h²+3a²)/6, where h=height of the tumour segment; a=(length+width of the tumour)/4; and V=volume of the tumour. Tumour tissue is retrieved, fixed in formalin (10%) and paraffin-embedded sections are prepared. Alternatively, Human A431 squamous cell carcinoma xenografts are established in athymic nude nu/nu mice, 6-8 weeks of age through subcutaneous inoculation of 0.5-2*10⁶ cells into the dorsal flank of each mouse. Administration of AMIGO-, AMIGO2- or AMIGO3 Ig-fusion protein or control FC-part protein (approx 10-40 mg/kg/dose) is done once daily to immunocompromised (athymic nude) mice upon injection of human A431 squamous cells. Tumours are measured at day 21 with calipers and the volume is calculates: V=π*h(h+3a²)/6, where h=height of the tumour segment; a=(length+width of the tumour)/4; and V=volume of the tumour. Tumour tissue is retrieved, fixed in formalin (10%) and paraffin-embedded sections are prepared.

Suppression of Tumorigenicity by Lentivirus-Mediated Gene Transfer of Soluble or Full Length AMIGO, AMIGO2 or AMIGO3

Animal experiments are adapted from Reed et al. (2002) Suppression of tumorigenicity by adenovirus-mediated gene transfer of decorin. Oncogene 21:3688-95. Human WiDr colon and A431 squamous cell carcinoma xenografts are established in athymic nude nu/nu mice, 6-8 weeks of age through subcutaneous inoculation of 0.5-2*10⁶ cells into the dorsal flank of each mouse. Mice are carefully examined every 2 or 3 days and any tumor growth is measured with a micro-caliper according to the following formula: V=a(b2/2), where a and b represent the larger and smaller diameters, respectively. When tumors reach 2-3 mm in greater diameter, each mouse receives direct intra-neoplastic injections and also three other injections 2, 4 and 6 days after first injection. The injections contain (approx 50 μl containing 4*10⁷ TU) replication-incompetent lentivirus, either empty virus or virus harboring the full-length AMIGO, AMIGO2 or AMIGO3 or soluble AMIGO, AMIGO2 or AMIGO3 ectodomain gene. Student's two-sided t-test is used to compare the values of the treated and control samples. A value of P<0.05 is considered as significant.

Animals are sacrificed at the end of the experiments, between 19 and 58 days depending on the treatment regimen and inoculum size, and each tumor is carefully dissected. The tumors are fixed in 10% buffered formaldehyde, embedded in paraffin and processed for routine histology. To determine the proliferative index of tumor xenografts, the percentage of tumor cell nuclei positive for Ki-67 marker is estimated in 10 high-power ('400) fields per animal.

Results

Idenfication and Cloning of a Novel Family of Transmembrane Proteins Containing a Tandem Array of Leucine-rich Repeats and an Immunoglobulin Domain (AMIGO, AMIGO2 and AMIGO3)

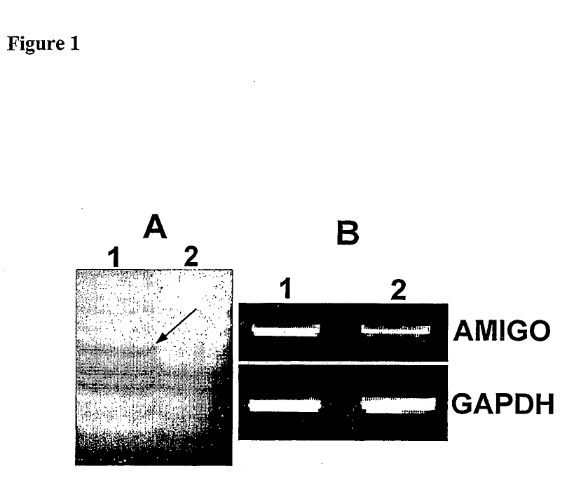

Ordered differential display (ODD; Matz et al., 1997) was used to search for amphoterin-induced genes in neurons. Comparison of ODD from embryonic day 18 rat hippocampal neurons grown on amphoterin and laminin coated plates revealed a transcript that was expressed more on amphoterin (FIG. 1 A). This expression difference was also confirmed with RT-PCR (FIG. 1 B).

The sequence of the partial transcript did not give homology with any previously cloned genes. By using the 5′ RACE method (Matz et al., 1999) the cDNA encoding the whole coding sequence was cloned (FIG. 2 A). We named this differentially expressed gene as AMIGO (AMphoterin Induced Gene and Orphan receptor). Hydrophobicity profile analysis (Nielsen et al., 1997; software SignalIP V2.0.b2) revealed that the protein sequence of AMIGO contains a putative signal sequence and a putative transmembrane region. The deduced extracellular part of the protein contains six leucine-rich repeats (LRRs) and one immunoglobulin domain. The deduced cytosolic part of the protein does not contain any known domains.

The human and mouse counterparts of AMIGO were also cloned with the 5′ RACE method by using data from the rat AMIGO sequence and from EST sequences. Identity at the amino acid level between the rat and mouse AMIGO is 95% and the murine sequences are 89% identical to the human AMIGO. In the extracellular part the most conserved motifs between the murine and human AMIGO are the N-terminal cysteine-rich domain and the LRRs 1-3. Interestingly, the whole transmembrane domain and the cytoplasmic tail are 100% identical between the murine and human AMIGO.

By using homology search we detected ESTs which gave homology but were not identical as compared to AMIGO. By using these EST sequences we cloned two other novel proteins which we named for convenience as AMIGO2 and AMIGO3. The deduced amino acid sequences show that AMIGO2 and AMIGO3 have the same domain organization as AMIGO: they also contain a putative signal seguence for secretion and six LRRs flanked on both the N and C-terminal sides by cysteine-rich LRRNT and LRRCT-domains. Like AMIGO, the deduced extracellular parts of AMIGO2 and AMIGO3 contain an immunoglobulin domain close to the transmembrane domain (for schematic picture of AMIGO, -2 and -3, see FIG. 2 B).

Similarity at the amino acid level between AMIGO to AMIGO2 is 48%, AMIGO to AMIGO3 is 50% and AMIGO2 to AMIGO3 is 48%. The alignment for AMIGO, -2 and -3 shows that the most conserved regions between the three proteins are the LRRs, the transmembrane region and some parts of the cytosolic tail (FIG. 2 A). The LRRs found in the AMIGOs can be described as a motif LX₂LXLX₂NX(L/I)X₂aX₄(F/L/I) (SEQ ID NO: 79) (in which “a” denotes an aliphatic residue and “X” any amino acid); this motif resembles a typical LRR sequence often found in extracellular parts of animal proteins (Kajava, 1998).

Expression of the Gene Family Members in Adult Tissues

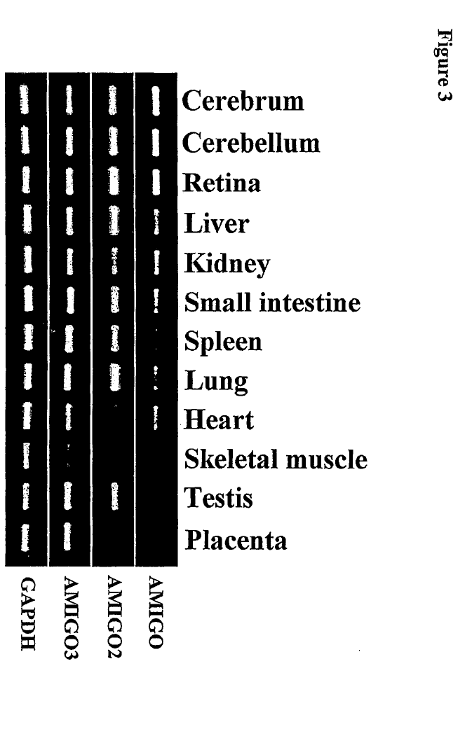

RT-PCR analysis of adult mouse tissues (FIG. 3) revealed that AMIGO is mainly expressed in the nervous tissues (cerebellum, cerebrum and retina) although some low expression could be also seen in liver, kidney, small intestine, spleen, lung and heart. AMIGO2 expression is most prominent in cerebellum, retina, liver and lung. A lower AMIGO2 mRNA expression is also seen in cerebrum, kidney, small intestine, spleen and testis. AMIGO3 mRNA expression could be detected in every tissue studied showing no specific expression pattern compared to AMIGO or AMIGO2. It thus appears that AMIGO is essentially a nervous system specific member of the protein family and we focused on AMIGO in more detail in the present study.

Cerebrum

In adult rat cerebrum the AMIGO staining was found from many nerve fiber bundles and nerve paths (FIGS. 7 and 9a). When compared to anti-CNPase staining, the AMIGO staining co-localizes with almost every myelinated areas of the cerebrum. In this study the only white matter area where AMIGO staining was absent was the lateral tractus olfactorius.

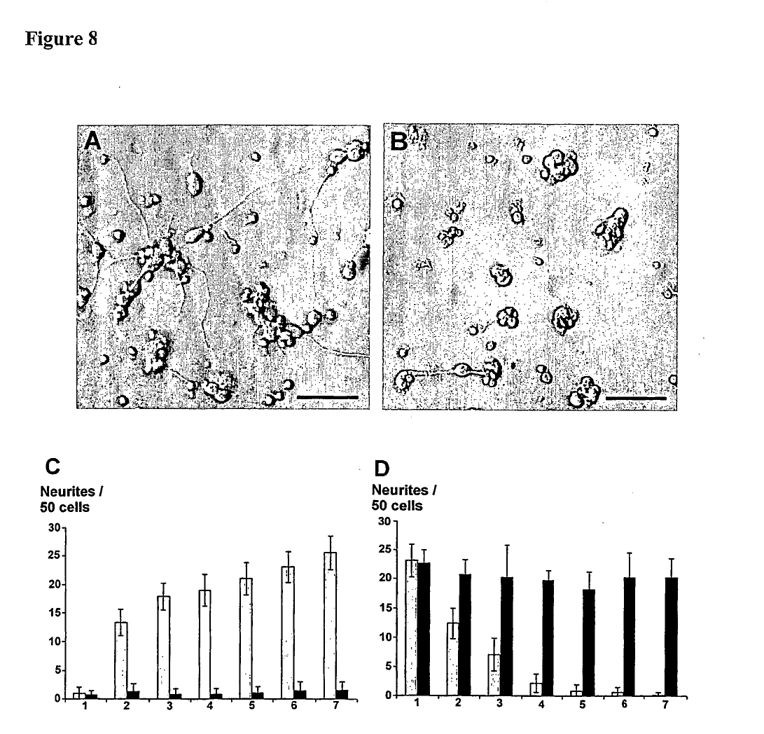

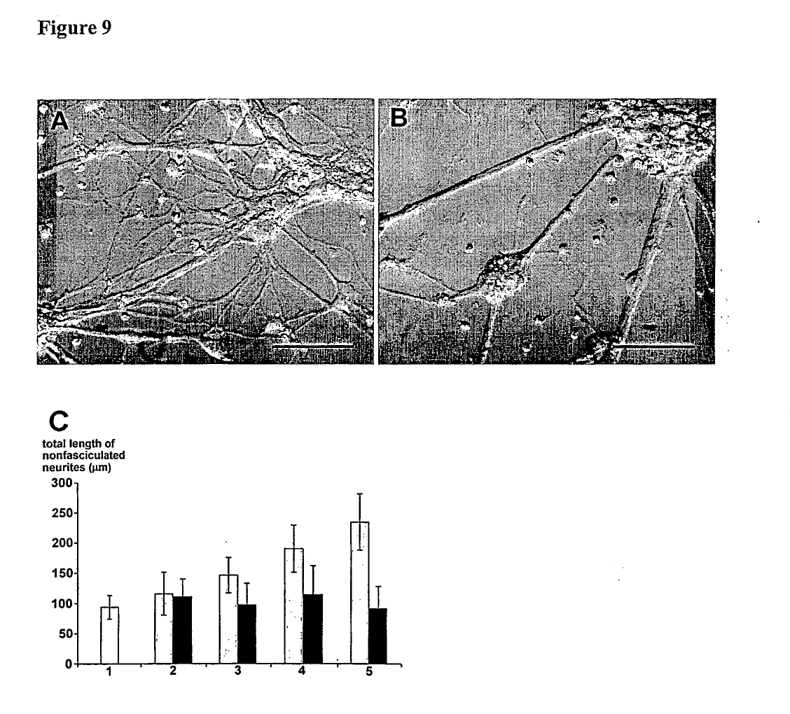

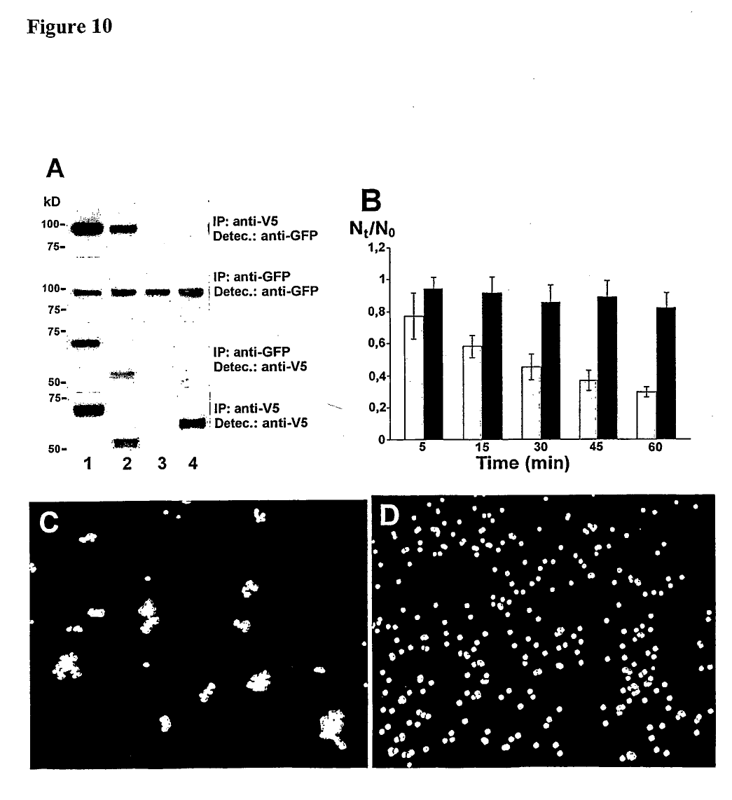

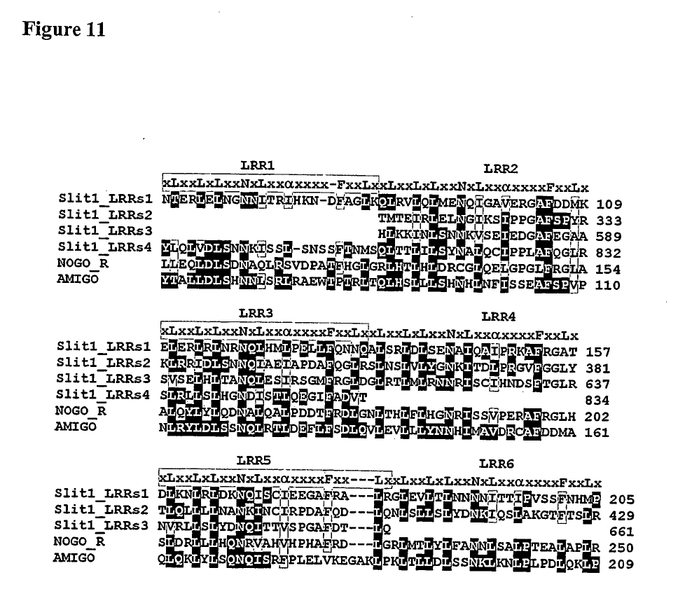

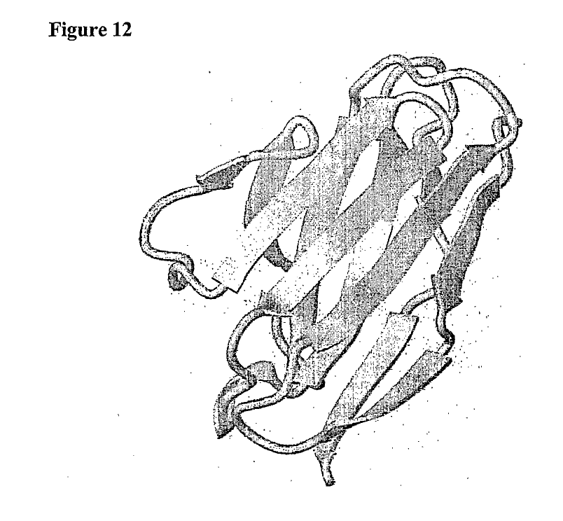

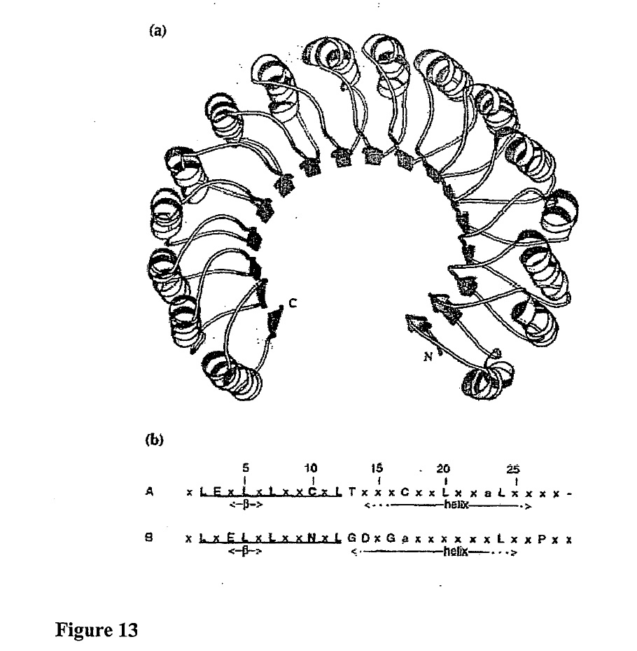

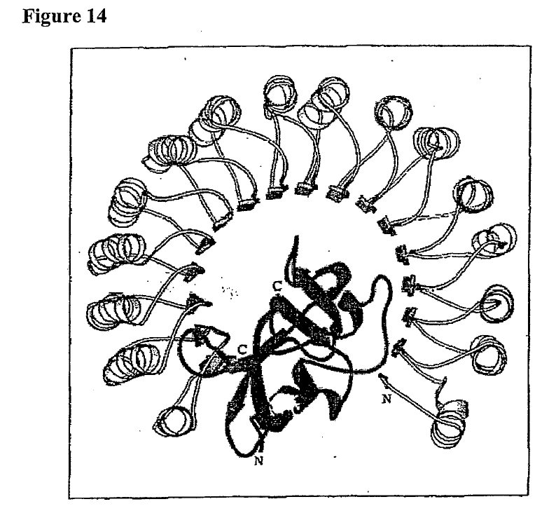

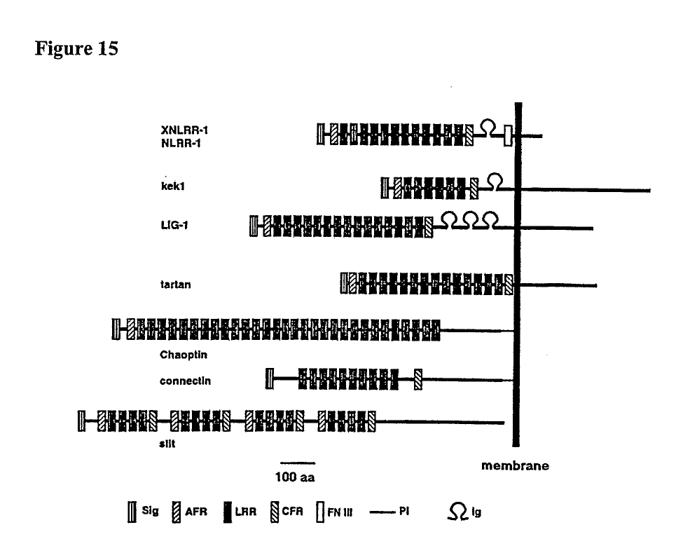

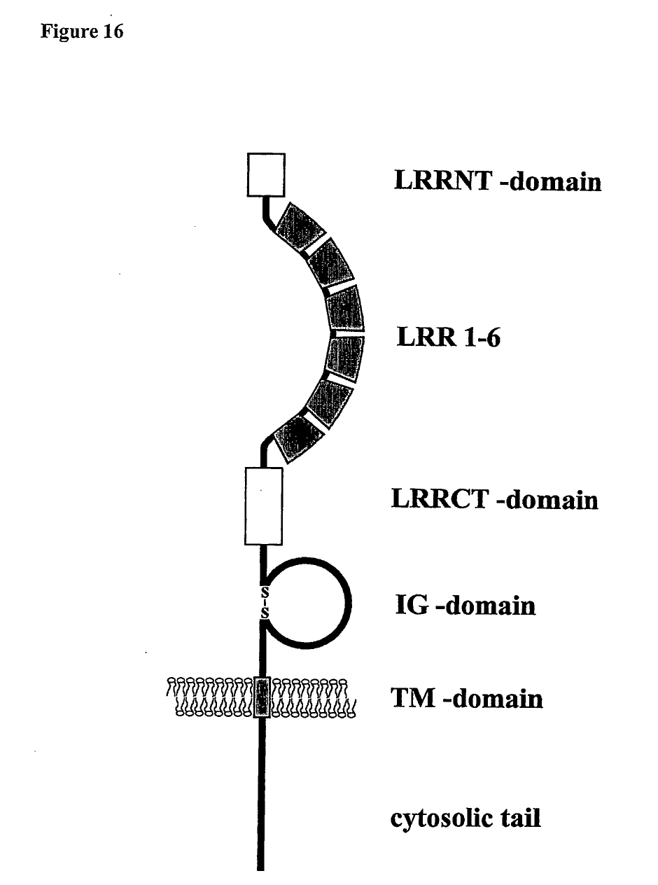

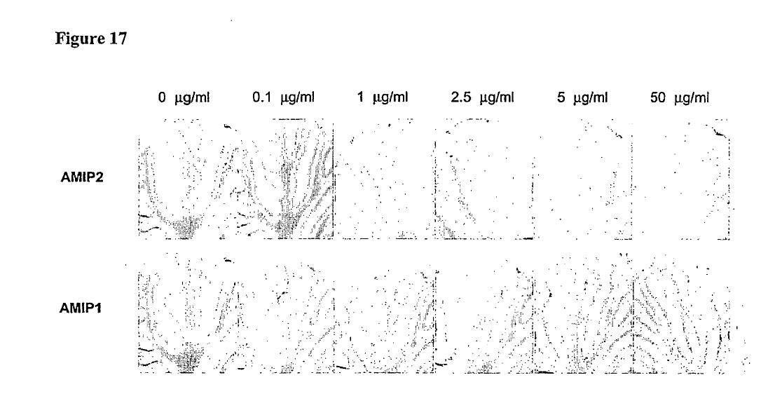

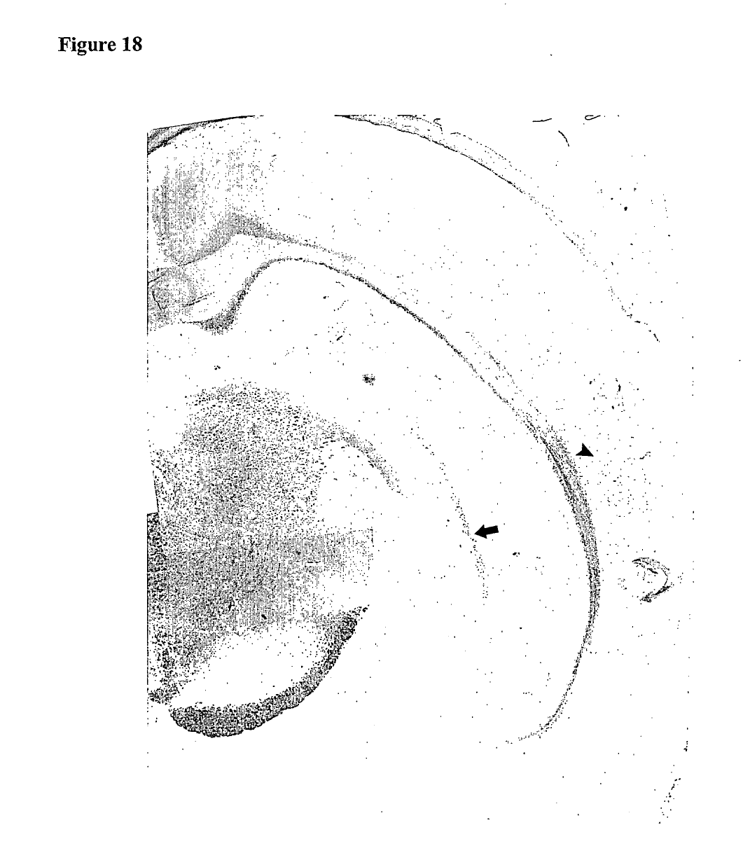

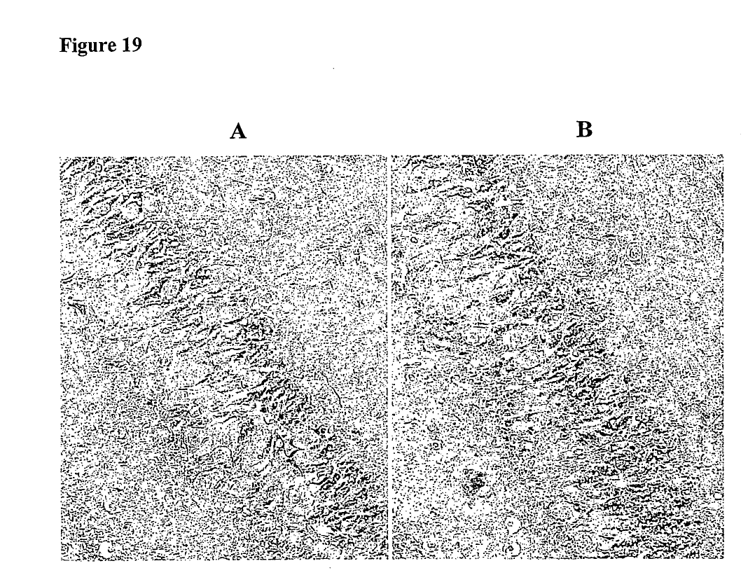

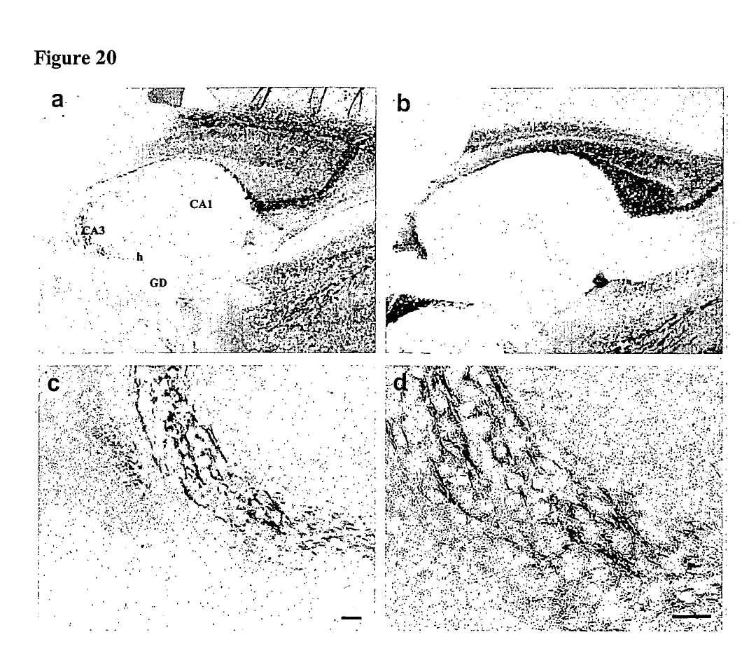

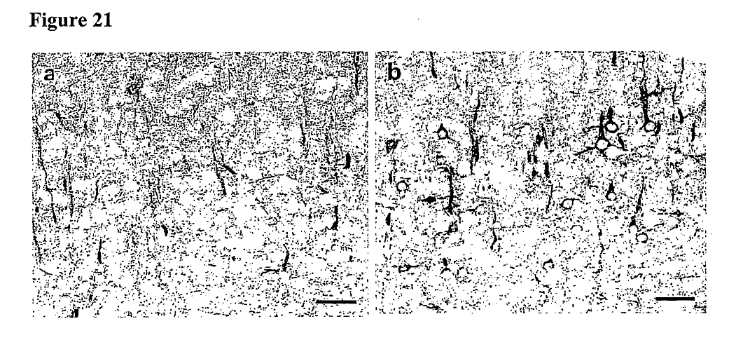

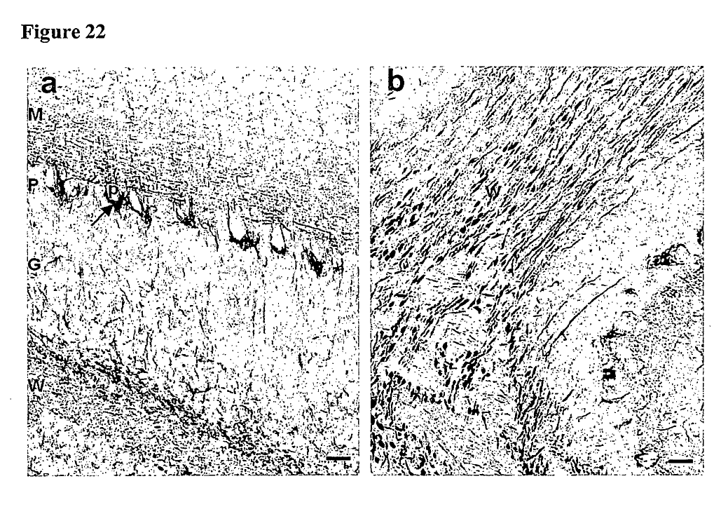

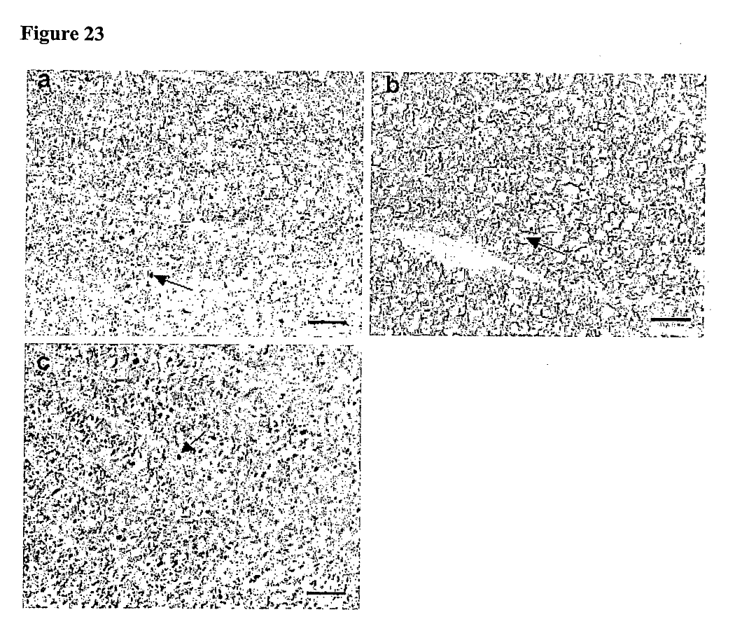

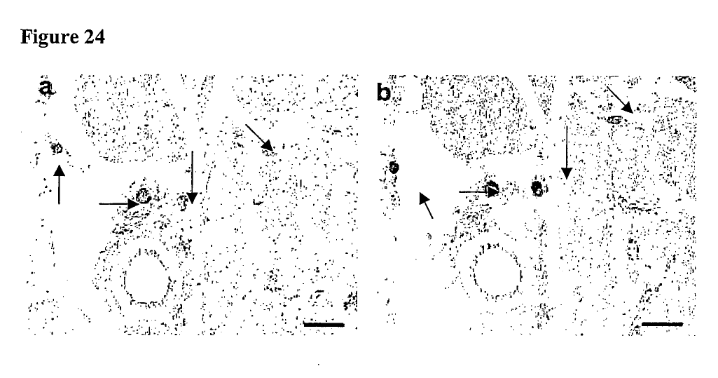

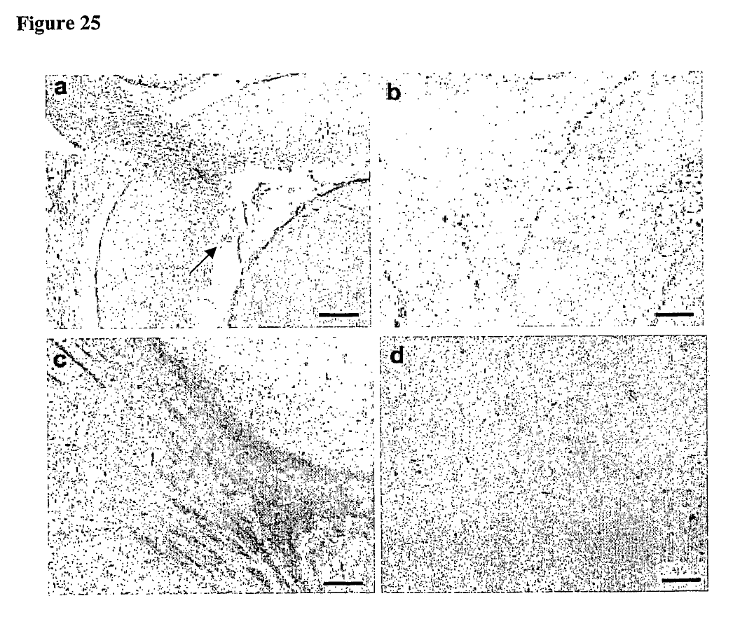

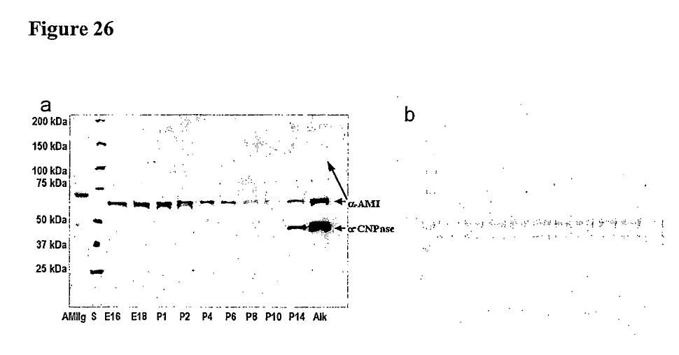

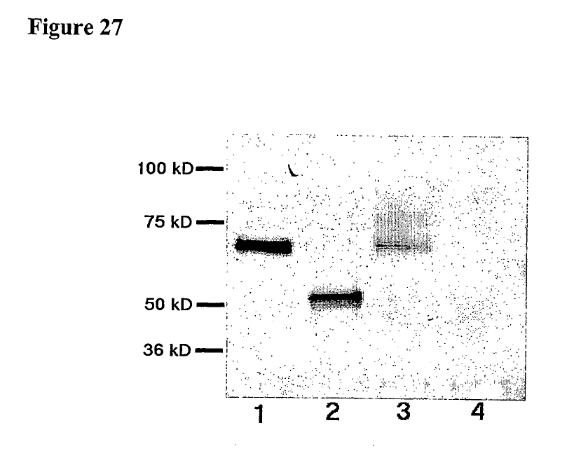

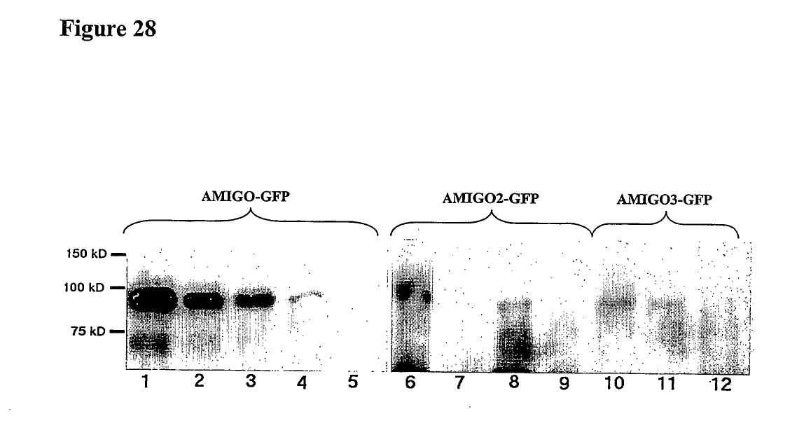

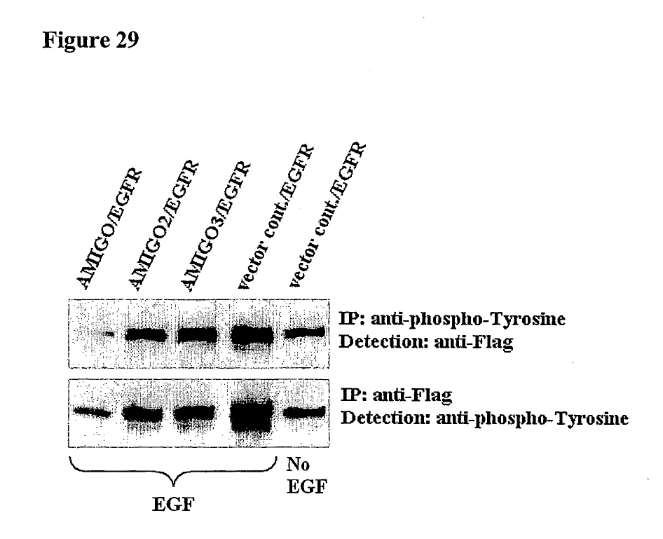

However, the AMIGO expression is not restricted to myelinated tracts; for example in hippocampus, non-myelinated tracts in the stratum lucidum CA3 region, which were negative for anti-CNPase and myelin basic protein (myelin basic protein data not shown), stained clearly with anti-AMIGO (FIGS. 9a and c). In coronal sections staining was restricted in the stratum lucidum of the CA3-region where it was localized more precisely in basal areas of the apical dendrites of the pyramidal cells (FIG. 8). The anti-AMIGO seemed to stain not the dendrites but the areas around the basal areas of the apical dendrites. In sagital sections the AMIGO staining was seen to be slightly fibrous (FIGS. 9c and d). The localization and structure of the AMIGO staining in hippocampus reminds the one seen for mossy-fibers. The mossy fibers are the axons of the granule cells from dentatum gyrus, which end up in the stratum lucidum of the CA3-region, where they form synapses with the apical dendrites of the pyramidal cells. The mossy-fibers have been shown to stain very intensively with anti-neurofilament antibodies (Huber et al., 1985).

Our anti-NF-M staining in hippocampus was very similar when compared to anti-AMIGO staining, which supports the interpretation that AMIGO localizes in mossy-fibers or structures very closely related to them. On the other hand these structures could be the interneuronal axons of the CA3-region, which have been shown to proceed along the mossy-fibers in stratum lucidum (Vida and Frotscher, 2000).

In cerebral cortex the AMIGO immunostaining was seen only in particular regions, which were also immunoreactive for anti-CNPase and anti-NF-M (FIG. 7). The cortical staining for all of the three antibodies used (AMIGO, CNPase and NF-M) was diffuse and indistinct, which is related in myelinated axons. At the same time the AMIGO staining is seen in the basal areas of the apical dendrites of the cortical pyramidal cells but interestingly not all of the apical dendrites are AMIGO immunoreactive. The anti-NF-M staining was also found in the apical dendrites but the staining could also be seen in the cell soma and the basal dendritic areas of the pyramidal cells (FIG. 10).

Cerebellum

In the cerebellum the anti-AMIGO staining was also co-localized with the anti-NF-M staining. In the cerebellum the anti-neurofilament antibodies have been seen to stain very intensively myelinated axons and basket cell axons (Matus et al., 1979).

The AMIGO staining was intensive in white matter and in the myelinated axons of the granular cell layer resembling the one seen for anti-NF-M. The most intensive staining in white matter was found in the middle of the cerebellum where the staining was seen in a string of pearls like structures (FIGS. 11a and b).

In the cortical areas of the cerebellum the AMIGO staining was seen in both sides of the Purkinje cell layer. The basket like structure around the Purkinje cell somas were seen to be immunoreactive for AMIGO and this structure is formed by the basket cell axons (FIG. 11a and b).

In the molecular layer of the cerebellum the AMIGO staining is seen in the fibers, which are orientated along the Purkinje cell layer (FIG. 11). At least some of these fibers are basket cell axons but also some other axons are AMIGO positive because the AMIGO immunostaining was more intensive when compared to anti-NF-M staining (data not shown).

Also the nuclei in the middle part of the cerebellum were AMIGO immunoreactive. In nuclei the AMIGO and NF-M staining differed form each other because AMIGO staining was only seen in neurites but NF-M staining could also be found from neurites and cell soma.

Pons and Medulla Oblongata

In pons and medulla oblongata the AMIGO staining was found in white matter.

Spinal Cord

In the cross-sections of the spinal cord the anti-AMIGO staining was seen in the white matter as a dotted like structures. In paraffin sections the myelin sheaths have melted away leaving round holes where the myelin has been located. In these sections the AMIGO staining is seen in the dots in the middle of the holes (FIG. 12a). Also the anti-NF-M antibodies stained these dots (FIG. 12c) whereas the anti-CNPase did not stained the same structures (FIG. 12b). In cryosections the AMIGO staining was seen to localize in the middle of the myelinated axons and not into the multilayered myelin sheaths (data not shown.). It is not clear whether all of the AMIGO positive axons were myelinated or not due to the limitations of the light microscopy.

In the grey matter of the spinal cord the anti-AMIGO stained some nerve fibers. Only some fibers of the grey matter, which were crossing into the white matter, were AMIGO positive. This suggests that AMIGO is expressed only in some subpopulation of these crossing axons (data not shown).

Kidney, Optic Nerve and Femoral Nerve

The AMIGO staining was found to co-localize with anti-NF-M staining in kidney. The stained structures were defined as autonomous nerve fibers (FIG. 13). The optic nerve was intensively stained with the anti-AMIGO antibodies whereas in femoral nerve the staining was absent (data not shown).

Embryos

In the head of the E18 rat embryo the staining was seen in nerve fibers and in nerve fiber tracts of internal capsule (FIG. 14 c), optic tract (FIG. 14a), middle cerebellar peduncle, stria medullaris, fasciculus retroflexus and longitudinal fasciculus pons. The AMIGO positive staining co-localized with anti-NF-M but the CNPase was not immunohistochemically detectable in E18 embryo (data not shown).

In the whole sections of the E16 embryo anti-AMIGO immunostaining was found only in some parts of the developing brain area, in optic nerve and areas close to the intestine and the rib bones (data not shown).

Expression of AMIGO During Development

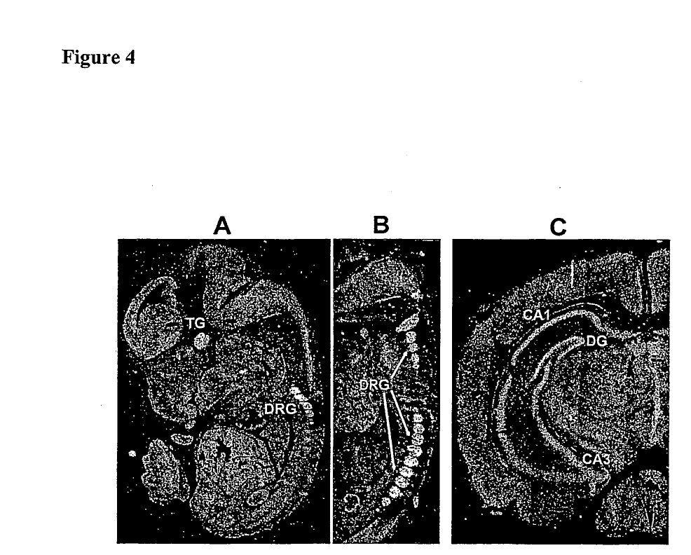

The AMIGO mRNA expression was studied in more detail using in situ hybridization. The AMIGO antisense probe gave a clear signal in the developing and adult nervous tissues whereas the sense probe did not give any clear signal (sense probe data not shown). A clear AMIGO expression was already detected in the E13 rodent embryo; at this stage the highest expression level was found in the dorsal root ganglia and the trigeminal ganglion with some expression in the central nervous system (FIG. 4 A-B). During later stages of development and in the adult, AMIGO was also prominently expressed in the brain, where the most intense signal was detected in the hippocampus (FIG. 4 C).

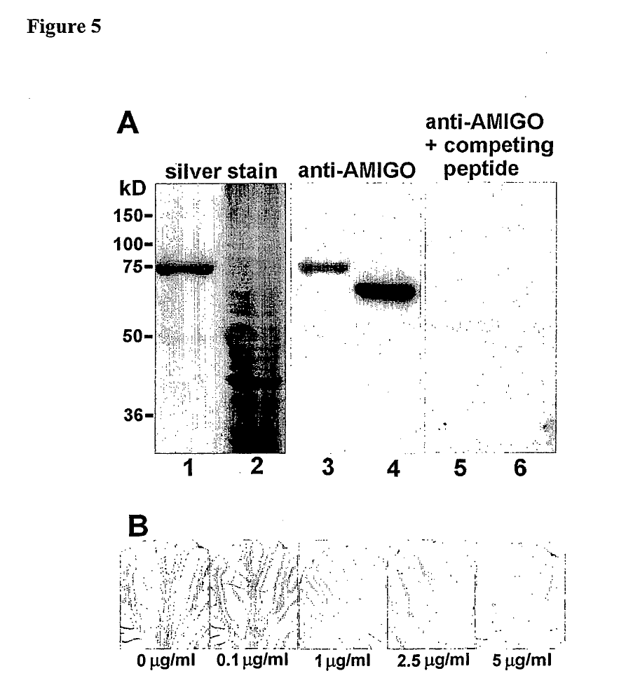

To investigate the expression of AMIGO at the protein level, polyclonal antisera were produced against an extracellular 10-amino acid peptide sequence that is found in AMIGO but not in AMIGO 2 or 3. The anti-peptide antibodies recognized the 75-kD AMIGO Ig-fusion protein produced in Drosophila S2 cells (FIG. 5, lanes 1 and 3). Western blotting of crude brain extracts revealed specific binding to a 65-kD polypeptide (FIG. 5, lanes 2 and 4). The molecular mass of the recognized polypeptide is close to the calculated molecular mass (56-kD) of AMIGO. Binding of the antibodies to both the fusion protein and the 65-kD polypeptide of brain were blocked by the synthetic peptide used as the immunogen (FIG. 5, lanes 3-6).

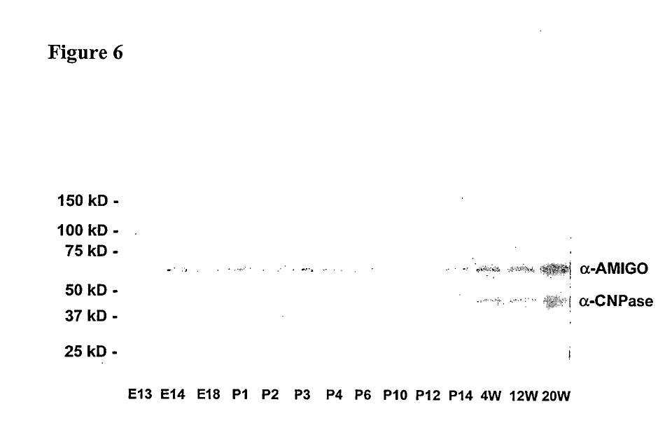

Western blotting of AMIGO using crude brain extracts from different developmental stages was consistent with the in situ hybridization data. The expression appears to start in the brain somewhat later than in the peripheral nervous system and increases clearly between E13 to E14 (FIG. 6). The expression is maintained high during the perinatal developmental stage but is downregulated during the postnatal stages P6 to P10. After this, the expression is again upregulated and remains high in the adult brain (FIG. 6). Since the time period of the postnatal upregulation of the AMIGO expression would appear to coincide with the onset of myelination, we compared the expression of AMIGO to that of the myelin-specific marker α-CNPase. Indeed, the expression of AMIGO and the CNPase display a parallel increase during postnatal development (FIG. 6). The AMIGO expression thus displays a dual character during brain development; the first expression peak occurs during the late embryonic and perinatal development, and the second increase in expression accompanies myelination.

Immunohistochemistry using the anti-peptide antibodies revealed specific staining only in the nervous system. In general, intensity of the immunostaining was in agreement with the expression data inferred from Western blotting (FIG. 6). Further, specificity of the immunostaining was suggested by inhibition of antibody binding to tissue sections by the peptide used as the immunogen (FIG. 5, panel B). In general, AMIGO was intensely stained in developing and mature fiber tracts. During embryonic development when the spinal ganglia express abundantly AMIGO mRNA (see FIG. 4), the immunostaining was observed in the fiber tracts connecting to the ganglia and the spinal cord but not in the ganglia themselves (FIG. 7, panel A), suggesting that the AMIGO protein is transported to axonal processes. In cerebellum, the most intense staining was observed in fibers on both sides of the Purkinje cell layer; the characteristic structure formed by the basket cell axons around the Purkinje cell soma was clearly discerned by the AMIGO immunostaining (FIG. 7 B). Consistent with the Western blotting data, AMIGO immunostaining labeled most myelinated axon tracts in the adult. An example is shown in FIG. 7 (panels C and D), demonstrating the similarity of the AMIGO and α-CNPase immunostaining around the hippocampus. However, the AMIGO expression is not restricted to myelinated tracts; for example in hippocampus, non-myelinated tracts in the stratum lucidum CA3 region, which were negative for α-CNPase (FIG. 7 D) and myelin basic protein (data not shown), stained clearly for AMIGO (FIG. 7 C). In general, AMIGO staining was detected (both during development and in adult animal) in large-diameter neurites (axons) that were also stained by antibodies against the 145 kD neurofilament (data not shown). As in the forebrain, myelinated axon tracts were also stained for AMIGO in cerebellum, pons, medulla and spinal cord.

AMIGO was also clearly immunostained both in the cell soma and in fasciculated and non-fasciculated processes of cultured hippocampal neurons (FIG. 7 F). As expected from immunostaining of tissue sections, double-immunostaining (not shown) revealed colocalization with the 145-kD neurofilament and the β-tubulin (TuJ1) but not with MAP2. AMIGO is thus preferentially expressed in axonal rather than dendritic processes.

AMIGO Promotes Neurite Extension of Hippocampal Neurons

Identification of AMIGO from hippocampal neurons growing neurites on amphoterin, the occurrence in fiber tracts in vivo and the domain structure with LRRs and Ig domains suggest that AMIGO might have a role in neurite extension. To get insight into the function of AMIGO, we tested if it is able to promote neurite outgrowth of hippocampal neurons. The extracellular part of the AMIGO was fused to human IgG Fc part, and this fusion protein was immobilized on microtiter wells and used as a substrate for hippocampal neurons. These experiments showed that the AMIGO Ig-fusion protein promotes attachment and neurite outgrowth of hippocampal neurons (FIG. 8 A and C), whereas on the human IgG Fc control neurite outgrowth was very low or undetectable figure (FIG. 8 B and C). Neurite outgrowth induced by the immobilized AMIGO Ig-fusion protein was inhibited by the soluble AMIGO Ig-fusion in the culture medium (FIG. 8 D).

Soluble AMIGO Perturbs Development of Fasciculated Axon Tracts In Vitro

Because AMIGO immunostaining could be found in vitro in hippocampal fasciculating axons and in the axon tracts in vivo, AMIGO might participate in fasciculation of neurites. We addressed this question by a dominant negative approach using the ectodomain of AMIGO as Ig-fusion protein in the culture medium. Hippocampal neurons were plated on poly-L-lysine coated wells to promote neurite outgrowth and fasciculation. Microscopy of the cultures revealed that the growth pattern of neurites was dramatically changed in the presence of the soluble AMIGO. In the control cultures neurites formed fascicles in 4 days, where as in the presence of the soluble AMIGO, the processes were mainly non-fasciculated up to at least 5 days in culture (FIG. 9 A-C).

AMIGO Displays a Homophilic Binding Mechanism

Fasciculation of axons is known to involve homophilic interactions and this might be reason why soluble AMIGO perturbs fasciculation. We therefore tested in a coimmunoprecipitation assay wether AMIGO could bind to itself. To examine AMIGO-AMIGO association, 293 cells were cotransfected with GFP-tagged full length AMIGO (FIG. 10 A, lanes 1-4) and V5-tagged full length AMIGO (FIG. 10 A, lane 1) and soluble V5-tagged AMIGO ectodomain (FIG. 10 A, lane 2). Immunoprecipitation of both AMIGO-V5 forms from the cell lysates precipitated AMIGO-GFP (FIG. 10 A, lanes 1 and 2) and correspondingly both the full length and soluble AMIGO-V5 were precipitated with anti-GFP (FIG. 10 A, lanes 1 and 2). No coimmunoprecipitation was observed when V5-tagged AMIGO was not transfected into cells (FIG. 10 A, lane 3). The control protein V5-tagged human RAGE was not coprecipitated with the AMIGO-GFP and vice versa. (FIG. 10 A, lane 3).

As another approach to study homophilic binding of AMIGO, we added AMIGO Ig-fusion protein to protein-A coated beads to get the protein oriented in a manner that occurs at the cell surface. AMIGO caused rapid aggregation of the beads (FIG. 10 B and C), whereas addition of the control protein IgG Fc part into the beads did not induce any aggregation (FIG. 10 B and D).

Coimmunoprecipitation of AMIGO and AMIGO2 with EGFR The result shows that both AMIGO and AMIGO2 bind the EGFR and only the EC-part is enough for the binding (shown for the AMIGO, FIG. 27).

AMIGO Inhibits EGFR Phosphorylation

When AMIGO and flag-tagged human EGFR are expressed together AMIGO could clearly inhibit the EGFR autophosphorylation induced by EGF ligation when compared to AMIGO2, AMIGO3 and vector control (FIG. 29).

Homo- and Heterophilic Binding of AMIGO, AMIGO2 and AMIGO3

The coimmunoprecipitation results show that AMIGOs could bind each others in heterophilically but they also posses homophilic binding properties (FIG. 28).

Discussion

A Novel Family of Transmembrane Proteins with Six LRR Domains and One Ig-Like Domain

In this study, we have identified a novel family of transmembrane proteins called AMIGO, AMIGO2 and AMIGO3. These three proteins show clear homology with each other; their length and location of different domains are highly identical (FIG. 2 B). This domain relationship suggests a common evolutionary origin of the AMIGOs.

Based on genomic sequence data these three proteins probably occur in the puffer fish Fugu rubripes (data not shown). Interestingly, Drosophila has a protein family called kekkon with three members of transmembrane proteins kek1, kek2 (Musacchio and Perrimon, 1996) and kek3 (Ashbumer et al., 1999) which show homology in their extracellular parts with the AMIGOs. The extracellular parts of both the AMIGOs and the kek proteins contain six LRR domains flanked with cysteine-rich LRRNT and LRRCT domains and one immunoglobulin domain close to the transmembrane region. However, the cytoplasmic parts of the AMIGOs and kek proteins do not display homology with each other. The gene expression data of kek1 and kek2 (Musacchio and Perrimon, 1996) reminds the one seen for AMIGO and AMIGO2; they all are expressed in the central nervous system of the adult organism. These domain and expression similarities suggest that the AMIGOs and kek proteins may be derived from a common ancestral gene.

In their extracellular parts the most homologous motifs between the AMIGOs are the LRRs 3-5. The best fit in BLAST searches shows homology with Slit family of extracellular axon-guiding proteins (Whitford et al 2002), and a clear homology is also found with the Nogo-66 receptor where the only recognizable motifs are the LRR domains (Fournier et al. 2001)(FIG. 11). The similarity found in the LRRs in AMIGO, Slit1 and Nogo-66 receptor suggests an evolutionary origin of these proteins from a common ancestor. The clear conservation seen at the LRR area between the AMIGOs suggests that this region is important for interactions with extracellular ligand(s) and that they could also share the same binding partner(s).

In the literature there are reports of other transmembrane proteins that contain LRRs and Ig domains in the extracellular part of the proteins: ISLR (Nagasawa et al., 1997): 5 LRRs and 1 Ig domain; Pal (Gomi et al., 2000): 5 LRRs and 1 Ig domain; LIG-1 (Suzuki et al., 1996): 15 LRRs and 3 Ig domains and GAC1 (Almeida et al., 1998): 12 LRRs and 1 Ig domain. Common for all of these proteins and the AMIGOs is the order of how the LRRs and the Ig domain(s) are organized; the LRRs are always more distal to the transmembrane region than the Ig domain(s). Interestingly, BLAST searches by using Ig-domain sequences from AMIGOs give no clear homology with other Ig-domains of the Ig-superfamily proteins but the most closest are the ones found in proteins containing both Ig and LRR domains (data not shown).

Although the cytoplasmic moieties of the AMIGOs do not display any clear homology with previously identified transmembrane proteins, the alignment of the AMIGOs (FIG. 2 A) shows two conserved serine-rich regions; one close to the transmembrane domain and the other at the C-terminus. The C-terminal serine-rich area of AMIGO and AMIGO2 have a consensus sequence for Casein kinase II (CK2) serine/threonine kinase (Allende et al. 1995) which is ubiquitously expressed in brain but AMIGO3, which is not expressed in the brain, does not have this consensus sequence. Recently Watts et al. (1999) showed that the transmembrane form of TNF-αα has a consensus sequence SXXS which is a substrate for Casein kinase 1 (CK1) dependent phosphorylation. Interestingly, all three AMIGOs have four possible CK1 phoshorylation sites in these two conserved serine rich areas. Future work will reveal whether these conserved serine residues have important functions in signalling events of the AMIGOs.

There are increasingly reports in the literature and the data banks on mammalian transmembrane proteins with both LRR and Ig domains but unfortunately at present almost all data only comprise the cloning and tissue expression of these proteins. Our data here gives a functional insight into these twin motif transmembrane proteins, belonging to both the LRR and Ig superfamily, in a form of more detailed characterization of AMIGO.

AMIGO, a Novel Transmembrane Protein in Neuronal Processes with Homophilic Binding Mechanism

Based on RT-PCR experiments, in situ hybridization and immunohistochemistry, AMIGO is an essentially nervous system specific protein. Interestingly, AMIGO expression is upregulated at two clearly distinct stages during brain development: the first peak is found perinatally, and the second upregulation occurs during or slightly before the upregulation of the oligodendrocyte-specific marker α-CNPase.

The first expression peak of AMIGO would be compatible with a role in growth of axonal connections. The expression of AMIGO in developing axon tracts both in vivo and in vitro and our neurite outgrowth experiments support this role. One cellular mechanism in the growth of axonal connections is fasciculation: axons grow along each other by using pioneer axons as the substratum for the growth cones of the later ones. Interestingly, a dominant negative approach using AMIGO ectodomain in the culture medium clearly suggests a role for AMIGO in fasciculation. Further, AMIGO displays a homophilic binding mechanism that would explain its role in fasciculation. Homophilic adhesion molecules belonging to both the Ig-superfamily and to the cadherin family have been shown to mediate neurite outgrowth and fasciculation during the nervous system development (for reviews, see Kamiguchi and Lemmon 1997; Martinek and Gaul 1997). It is also noteworthy that the LRR sequences of the AMIGOs display homology with the slit proteins and with the Nogo receptor (FIG. 11) that have been implicated in axon growth, regeneration and guidance.

The second upregulation of the AMIGO expression suggests a role in myelination. It seems reasonable that AMIGO would mediate cell-to-cell interactions also at this stage of development. However, further studies are clearly warranted to understand the role of AMIGO in myelinating axon tracts, like in the interactions of axons with oligodendrocytes and Schwann cells. Further, AMIGO expression remains high until adulthood. This suggests that AMIGO plays a role in regeneration and plasticity of the adult fiber tracts, the mechanisms of which commonly recapitulate mechanisms of fiber tract development.

To get further insight into the functional roles of AMIGO during development and adulthood, we have recently targeted the gene in ES cells and are currently producing AMIGO null mice (Kuja-Panula and Rauvala, unpublished results). In addition to the in vivo approaches using gene targeting, it will be important to understand what molecular domains mediate homophilic binding and whether the intracellular domain of AMIGO has signalling properties. Furthermore, future studies will reveal whether the members of the AMIGO family mediate analogous cell-to-cell interactions in non-neuronal tissues characterized in the present paper for AMIGO in axonal tracts.

It will be appreciated that the methods of the present invention can be incorporated in the form of a variety of embodiments, only a few of which are disclosed herein. It will be apparent for the specialist in the field that other embodiments exist and do not depart from the spirit of the invention. Thus, the described embodiments are illustrative and should not be construed as restrictive.

The publications and other materials used herein to illuminate the background of the invention, and in particular, to provide additional details with respect to its practice, are incorporated herein by reference.

### REFERENCE LIST FOR EXPERIMENTAL SECTION

Agarwala, K. L., S. Ganesh, Y. Tsutsumi, T. Suzuki, K. Amano, and K. Yamakawa. 2001. Cloning and functional characterization of DSCAML1, a novel DSCAM-like cell adhesion molecule that mediates homophilic intercellular adhesion. Biochem. Biophys. Res. Commun. 285:760-772.
Allende, J. E. and C. C. Allende. 1995. Protein kinases. 4. Protein kinase CK2: an enzyme with multiple substrates and a puzzling regulation. FASEB. J. 9:313-323.
Almeida, A., X. X. Zhu, N. Vogt, R. Tyagi, M. Muleris, A. M. Dutrillaux, B. Dutrillaux, D. Ross, B. Malfoy, and S. Hanash. 1998. GAC1, a new member of the leucine-rich repeat superfamily on chromosome band 1q32.1, is amplified and overexpressed in malignant gliomas. Oncogene. 16:2997-3002.
Ashburner, M., S. Misra, J. Roote, S. E. Lewis, R. Blazej, T. Davis, C. Doyle, R. Galle, R. George, N. Harris, G. Hartzell, D. Harvey, L. Hong, K. Houston, R. Hoskins, G. Johnson, C. Martin, A. Moshrefi, M. Palazzolo, M. G. Reese, A. Spradling, G. Tsang, K. Wan, K. Whitelaw, and S. Celniker. 1999. An exploration of the sequence of a 2.9-Mb region of the genome of Drosophila melanogaster: the Adh region. Genetics. 153:179-219.
Battye, R., A. Stevens, and J. R. Jacobs. 1999. Axon repulsion from the midline of the Drosophila CNS requires slit function. Development. 126:2475-2481.
Battye, R., A. Stevens, R. L. Perry, and J. R. Jacobs. 2001. Repellent signaling by Slit requires the leucine-rich repeats. J. Neurosci. 21:4290-4298.
Brose, K., K. S. Bland, K. H. Wang, D. Arnott, W. Henzel, C. S. Goodman, M. Tessier-Lavigne, and T. Kidd. 1999. Slit proteins bind Robo receptors and have an evolutionarily conserved role in repulsive axon guidance. Cell. 96:795-806.
Chen, M. S., A. B. Huber, M. E. van der Haar, M. Frank, L. Schnell, A. A. Spillmann, F. Christ, and M. E. Schwab. 2000. Nogo-A is a myelin-associated neurite outgrowth inhibitor and an antigen for monoclonal antibody IN-1. Nature. 403: 434-439.
Drescher, U., A. Faissner, R. Klein, F. G. Rathjen, and C. Sturmer. 1997. Axonal growth and pathfinding: from phenomena to molecules. Cell Tissue Res. 290:187-188.
Fournier, A. E., T. GrandPre, and S. M. Strittmatter. 2001. Identification of a receptor mediating Nogo-66 inhibition of axonal regeneration. Nature. 409:341-346.
Gomi, F., K. Imaizumi, T. Yoneda, M. Taniguchi, Y. Mori, K. Miyoshi, J. Hitomi, T. Fujikado, Y. Tano, and M. Tohyama. 2000. Molecular cloning of a novel membrane glycoprotein, pal, specifically expressed in photoreceptor cells of the retina and containing leucine-rich repeat. J. Neurosci. 20:3206-3213.
Kajava, A. V. 1998. Structural diversity of leucine-rich repeat proteins. J. Mol. Biol. 277:519-527.
Kamiguchi, H. and V. Lemmon. 1997. Neural cell adhesion molecule L1: signaling pathways and growth cone motility. J. Neurosci. Res. 49:1-8.
Logan, A., M. Berry, A. M. Gonzalez, S. A. Frautschy, M. B. Sporn, and A. Baird. 1994. Effects of transforming growth factor b1 on scar production in the injured central nervous system. Eur. J. Neurosci. 6: 355-363.
Martinek, S. and U. Gaul. 1997. Neural development: how cadherins zipper up neural circuits. Curr. Biol. 7:R712-R715
Matz, M., N. Usman, D. Shagin, E. Bogdanova, and S. Lukyanov. 1997. Ordered differential display: a simple method for systematic comparison of gene expression profiles. Nucleic Acids Res. 25:2541-2542.
Muller, S., P. Scaffidi, B. Degryse, T. Bonaldi, L. Ronfani, A. Agresti, M. Beltrame, and M. E. Bianchi. 2001. New EMBO members' review: the double life of HMGB1 chromatin protein: architectural factor and extracellular signal. EMBO. J. 20:4337-4340.
Musacchio, M. and N. Perrimon. 1996. The Drosophila kekkon genes: novel members of both the leucine-rich repeat and immunoglobulin superfamilies expressed in the CNS. Dev. Biol. 178:63-76.
Nagasawa, A., R. Kubota, Y. Imamura, K. Nagamine, Y. Wang, S. Asakawa, J. Kudoh, S. Minoshima, Y. Mashima, Y. Oguchi, and N. Shimizu. 1997. Cloning of the cDNA for a new member of the immunoglobulin superfamily (ISLR) containing leucine-rich repeat (LRR). Genomics. 44:273-279.
Nielsen, H., J. Engelbrecht, S. Brunak, and G. von Heijne. 1997. Identification of prokaryotic and eukaryotic signal peptides and prediction of their cleavage sites. Protein Eng. 10: 1-6.
Pusch, C. M., C. Zeitz, O. Brandau, K. Pesch, H. Achatz, S. Feil, C. Scharfe, J. Maurer, F. K. Jacobi, A. Pinckers, S. Andreasson, A. Hardcastle, B. Wissinger, W. Berger, and A. Meindl. 2000. The complete form of X-linked congenital stationary night blindness is caused by mutations in a gene encoding a leucine-rich repeat protein. Nat. Genet. 26:324-327.
Rauvala, H. and R. Pihlaskari. 1987. Isolation and some characteristics of an adhesive factor of brain that enhances neurite outgrowth in central neurons. J. Biol. Chem. 262:16625-16635.
Rauvala, H., H. J. Huttunen, C. Fages, M. Kaksonen, T. Kinnunen, S. Imai, E. Raulo, and I. Kilpelainen. 2000. Heparin-binding proteins HB-GAM (pleiotrophin) and amphoterin in the regulation of cell motility. Matrix Biol. 19:377-387.
Reponen, P., C. Sahlberg, C. Munaut, I. Thesleff, and K. Tryggvason. 1994. High expression of 92-kD type IV collagenase (gelatinase B) in the osteoclast lineage during mouse development. J. Cell Biol. 124:1091-1102.
Schachner, M. 1997. Neural recognition molecules and synaptic plasticity. Curr. Opin. Cell Biol. 9:627-34
Stoeckli, E. T. and L. T. Landmesser. 1998. Axon guidance at choice points. Curr. Opin. Neurobiol. 8:73-79.
Suzuki, Y., N. Sato, M. Tohyama, A. Wanaka, and T. Takagi. 1996. cDNA cloning of a novel membrane glycoprotein that is expressed specifically in glial cells in the mouse brain. LIG-1, a protein with leucine-rich repeats and immunoglobulin-like domains. J. Biol. Chem. 271:22522-22527.
Tessier-Lavigne, M. and C. S. Goodman. 1996. The molecular biology of axon guidance. Science. 274:1123-1133.
Van Vactor, D. 1998. Adhesion and signaling in axonal fasciculation. Curr. Opin. Neurobiol. 8:80-86.
Walsh, F. S. and P. Doherty. 1997. Neural cell adhesion molecules of the immunoglobulin superfamily: role in axon growth and guidance. Annu. Rev. Cell Dev. Biol. 13:425-456.
Watts, A. D., N. H. Hunt, Y. Wanigasekara, G. Bloomfield, D. Wallach, B. D. Roufogalis, and G. Chaudhri. 1999. A casein kinase I motif present in the cytoplasmic domain of members of the tumour necrosis factor ligand family is implicated in ‘reverse signalling’. EMBO. J. 18:2119-2126.
Whitford, K. L., V. Marillat, E. Stein, C. S. Goodman, M. Tessier-Lavigne, A. Chedotal, and A. Ghosh. 2002. Regulation of cortical dendrite development by Slit-Robo interactions. Neuron. 33:47-61.

1. A purified and isolated AMIGO nucleic acid comprising a nucleotide sequence that encodes a polypeptide comprising the amino acid sequence shown in SEQ ID NO:2, 4 or 6.

2. A purified and isolated nucleic acid comprising a nucleotide sequence shown in SEQ ID NO:1, 3 or 5.

3. A purified and isolated nucleic acid comprising a recombinant nucleotide sequence comprising a nucleotide sequence shown in SEQ ID NO:1, 3 or 5 or a homolog or fragment thereof.

4. An expression construct comprising the nucleic acid according to claim 2 operatively linked to an expression control sequence, said expression construct capable of encoding an AMIGO polypeptide or variants thereof.

5. A host cell transformed or transfected with the expression construct of claim 4.

6. A host cell transformed or transfected with a polynucleotide wherein said polynucleotide includes a strand containing a human nucleotide sequence that hybridizes to a DNA comprising the non-coding strand complementary to SEQ ID NO:1, 3 or 5, under the following hybridization conditions:
(a) hybridization at 42° C. for 20 hours in a solution containing 50% formamide, 5×SSPE, 5× Denhardt's solution, 0.1% SDS and 0.1 mg/ml denatured salmon sperm DNA; and
(b) washing the filter twice for thirty minutes at room temperature and twice for thirty minutes at 65° C. with a wash solution containing 1×SSC, and 0.1% SDS.

7. An isolated and purified AMIGO polypeptide comprising the amino acid sequence of SEQ ID NO:2, 4 or 6.

8. Method of producing an AMIGO polypeptide according to claim 7, said method comprising the steps of:
culturing a host cell of claim 5 comprising a polynucleotide encoding said polypeptide operably associated with a promoter sequence such that the nucleic acid sequence encoding said polypeptide is expressed; and
isolating said polypeptide from said host cell or from a growth medium in which said host cell is cultured.

9. Method of producing antibodies comprising:
immunising a mammal with the isolated and purified AMIGO protein of claim 7 or an antigenic fragment thereof.

10. Use of the isolated and purified AMIGO protein of claim 7 or an antigenic fragment thereof as an antigen.

11. An antibody produced by the method of claim 9.

12. The antibody of claim 11 which is labeled with a detectable label.

13. A kit of reagents for use in detecting the presence of AMIGO or allelic variant thereof in a biological sample, comprising
a container; and in said container:
a compound, preferably labeled, capable of detecting AMIGO or allelic variants thereof.

14. The kit according to claim 13, wherein said compound is a primer or probe.

15. The kit according to claim 13, wherein said compound is an antibody as defined in claim 11.

16. The kit according to claim 13 for assessing the predisposition of an individual to a condition mediated by variation or dysfunction of AMIGO.

17. The kit according to claim 16 further comprising instructions for using the kit.

18. A transgenic non-human animal containing a human or murine AMIGO gene as a transgene.

19. A transgenic non-human animal containing a transgene or insertion disrupting expression of an AMIGO gene or a homolog thereof.

20. A pharmaceutical compound comprising AMIGO nucleic acid molecule, AMIGO protein, AMIGO peptide fragment, AMIGO fusion protein, AMIGO agonists, AMIGO antagonists or anti-AMIGO antibody.

21. Method for treatment of a condition dependent on AMIGO wherein a pharmaceutically effective amount of the compound of claim 20 is administered to a patient in need of such treatment.

22. Method for affinity purification of ligand that binds to the AMIGO comprising the following steps: a) contacting a source of AMIGO receptor with an immobilized AMIGO under conditions whereby the AMIGO receptor to be purified is selectively adsorbed onto the immobilized AMIGO; (b) washing the immobilized AMIGO and its support to remove non-adsorbed material; and (c) eluting the AMIGO receptor molecules from the immobilized AMIGO to which they are adsorbed with an elution buffer.

23. A method for identifying a modulator of binding between an AMIGO receptor and an AMIGO receptor, comprising steps of:
(a) contacting an AMIGO receptor composition with an AMIGO composition in the presence and in the absence of a putative modulator compound;
(b) detecting binding between AMIGO receptor and the AMIGO receptor in the presence and absence of the putative modulator; and
(c) identifying a modulator compound in view of decreased or increased binding between the AMIGO receptor and the AMIGO receptor in the presence of the putative modulator, as compared to binding in the absence of the putative modulator.

24. A method according to claim 23, further comprising a step of:
(d) making a modulator composition by formulating a modulator identified according to step (c) in a pharmaceutically acceptable carrier.

25. A method according to claim 24, further comprising a step of:
(e) administering the modulator composition to an animal that comprises cells that express the AMIGO receptor, and determining physiological effects of the modulator composition in the animal.

26. A method according to claim 23, wherein the AMIGO receptor composition comprises a member selected from the group consisting of:
(a) a purified polypeptide comprising a AMIGO receptor extracellular domain fragment that binds the AMIGO;
(b) a phospholipid membrane containing AMIGO receptor polypeptides; and
(c) a cell recombinantly modified to express increased amounts of an AMIGO receptor on its surface.

27. A method according to claim 23, wherein the AMIGO receptor composition comprises an AMIGO receptor extracellular domain fragment bound to a solid support.

28. A method according to claim 23, wherein the AMIGO receptor composition comprises an AMIGO receptor extracellular domain fragment fused to an immunoglobulin Fc fragment.

29. A method according to claim 23, wherein the AMIGO receptor is selected from the group consisting of a mammalian AMIGO, AMIGO2, and AMIGO3.

30. A method according to claim 23, wherein the AMIGO receptor is human.

31. A method according to claim 23, wherein the AMIGO composition comprises a member selected from the group consisting of:
(a) a purified polypeptide comprising an AMIGO fragment that binds the AMIGO receptor;
(b) a phospholipid membrane containing AMIGO polypeptides; and
(c) a cell recombinantly modified to express increased amounts of an AMIGO on its surface.

32. A method according to claim 23, wherein the AMIGO composition comprises an AMIGO extracellular domain fragment bound to a solid support.

33. A method according to claim 23, wherein the AMIGO composition comprises an AMIGO extracellular domain fragment fused to an immunoglobulin Fc fragment.

34. A method according to claim 23, wherein the AMIGO is human.

35. A method according to claim 23, wherein the AMIGO receptor composition comprises a cell recombinantly modified to express increased amounts of an AMIGO receptor on its surface, and wherein the detecting step comprises measuring an AMIGO binding-induced physiological change in the cell.

36. A method according to claim 23, wherein the AMIGO composition comprises a cell recombinantly modified to express increased amounts of an AMIGO on its surface, and wherein the detecting step comprises measuring an AMIGO binding-induced physiological change in the cell.

37. A method for screening for selectivity of a modulator of binding between an AMIGO and an EGFR, comprising steps of:
a) contacting an AMIGO receptor composition with an EGFR composition in the presence and in the absence of a compound that modulates binding between the AMIGO receptor and EGFR receptor; and
b) detecting binding between the AMIGO receptor composition and the EGFR receptor composition in the presence and absence of the modulator compound,
c) identifying the selectivity of the modulator compound in view of decreased or increased binding between the AMIGO receptor and the EGFR receptor in the presence as compared to the absence of the modulator, wherein increased selectivity of the modulator for modulating AMIGO EGFR binding correlates with decreased differences in AMIGO-EGFR binding.

38. A method of modulating growth, migration, axonal growth, myelination, fasciculation or proliferation of cells in a mammalian organism, comprising a step of:
(a) identifying a mammalian organism having cells that express a AMIGO receptor and/or EGFR; and
(b) administering to said mammalian organism a composition, said composition comprising an agent selected from the group consisting of:
(i) a polypeptide comprising an AMIGO receptor that binds to the AMIGO receptor and/or EGFR, or a nucleic acid encoding said polypeptide;
(ii) a polypeptide comprising a fragment of the AMIGO, wherein the polypeptide and fragment retain AMIGO binding characteristics of the AMIGO, or a nucleic acid encoding said polypeptide;
(iii) an antibody that specifically binds the polypeptide of (i) or (ii) in a manner that inhibits the polypeptide from binding the AMIGO receptor and/or EGFR, or a fragment of the antibody that specifically binds the polypeptide of (i) or (ii);
(iv) a polypeptide comprising an antigen-binding fragment of (iii) and that inhibits the polypeptide of (i) or (ii) from binding the AMIGO receptor and/or EGFR;
(v) a molecule that selectively inhibits AMIGO binding to the AMIGO receptor without inhibiting AMIGO binding to the EGFR receptor; and
(vi) a molecule selectively binding to the AMIGO receptor and the EGFR receptor;
wherein the composition is administered in an amount effective to modulate growth, migration, or proliferation of cells that express AMIGO in the mammalian organism.

39. A method according to claim 38, wherein the mammalian organism is human.

40. A method according to claim 38, wherein the cells comprise neuronal cells.

41. A method according to claim 38, wherein the organism has a disease characterized by aberrant growth, migration, or proliferation of neuronal cells/neuronal extensions.

42. A method according to claim 38, wherein the conditions comprises a neuronal trauma.

43. A method according to claim 38, further comprising administering a second agent to the patient for modulating neuronal growth, migration, regeneration or proliferation, said second agent selected from the group consisting of: an antibody that specifically binds with any of the foregoing polypeptides, an antibody that specifically binds with a receptor for any of the foregoing polypeptides, or a polypeptide comprising an antigen binding fragment of such antibodies.

44. A method according to claim 38, wherein the AMIGO extracellular fragment is conjugated with Fc domain.

45. A method according to claim 44, wherein rat AMIGO Fc fusion protein sequences have been replaced essentially with the human AMIGO and Fc sequences

46. A polypeptide according to claim 38, for use in the manufacture of a medicament for the treatment of diseases characterized by aberrant growth, migration, regeneration or proliferation of cells that express an AMIGO receptor.

47. Method according to claim 38 wherein neuronal cells are selected from the group consisting of: hippocampal cells, cerebral cells, cerebellar cells, neuronal trauma cells, glial scar cells, spinal cord cells, optic nerve cells, retina cells, kidney cells, and cells acting during fasciculation, guidance, growth, or myelination.

48. A method of modulating cancer, tumour growth or metastasis in a mammalian organism, comprising a step of:
(a) identifying a mammalian organism having cells that express an AMIGO receptor and/or EGFR; and
(b) administering to said mammalian organism a composition, said composition comprising an agent selected from the group consisting of:
(i) a polypeptide comprising an AMIGO receptor that binds to the AMIGO receptor and/or EGFR, or a nucleic acid encoding said polypeptide;
(ii) a polypeptide comprising a fragment of the AMIGO, wherein the polypeptide and fragment retain AMIGO binding characteristics of the AMIGO, or a nucleic acid encoding said polypeptide;
(iii) an antibody that specifically binds the polypeptide of (i) or (ii) in a manner that inhibits the polypeptide from binding the AMIGO receptor and/or EGFR, or a fragment of the antibody that specifically binds the polypeptide of (i) or (ii);
(iv) a polypeptide comprising an antigen-binding fragment the (iii) and that inhibits the polypeptide of (i) or (ii) from binding the AMIGO receptor and/or EGFR;
(v) a molecule that selectively inhibits AMIGO binding to the AMIGO receptor without inhibiting AMIGO binding to the EGFR receptor; and
(vi) a molecule selectively binding to the AMIGO receptor and the EGFR receptor;
wherein the composition is administered in an amount effective to modulate cancer growth or metastasis of cells that express AMIGO in the mammalian organism.

49. A method according to claim 48, wherein the mammalian organism is human.

50. A method according to claim 48 or 49, wherein the cells comprise glioma, glioblastoma, astrocytoma, anaplastic astrocytoma, ependymomas, oligodendrogliomas, medulloblastomas, meningiomas, schwannomas, craniopharyngiomas, germ cell tumors, pineoblastoma, pineocytoma, germinoma cells, lung carcinoma, breast carcinoma, ovarian carcinoma, colorectal carcinoma, bladder carcinoma, pancreatic carcinoma, squamous cell carcinoma, or renal carcinoma cells.

51. A method according to claim 48, wherein the organism has a disease characterized by cancer or metastasis.

52. A method according to claim 51, wherein the condition comprises a brain tumor.

53. A method according to claim 48, further comprising administering a second agent to the patient for modulating cancer growth or metastatic growth of cancer, said second agent selected from the group consisting of: an antibody that specifically binds with any of the foregoing polypeptides, an antibody that specifically binds with a receptor for any of the foregoing polypeptides, or a polypeptide comprising an antigen binding fragment of such antibodies.

54. A method according to claim 48, wherein the AMIGO extracellular fragment is conjugated with Fc domain.

55. A method according to claim 48, wherein rat AMIGO Fc fusion protein sequences have been replaced essentially with the human AMIGO and Fc sequences

56. Method for treatment of cancer or metastatic growth of cancer cells selected from the group consisting of: glioma, glioblastoma, astrocytoma, anaplastic astrocytoma, ependymomas, oligodendrogliomas, medulloblastomas, meningiomas, schwannomas, craniopharyngiomas, germ cell tumors of germinoma cells, lung carcinoma, breast carcinoma, ovarian carcinoma, colorectal carcinoma, bladder carcinoma, pancreatic carcinoma, squamous cell carcinoma, and renal carcinoma, comprising a step of administering to a subject in need of such treatment the compound as claimed in claim 20.

57. Method for treatment of neuronal cells selected from the group consisting of: hippocampal cells, cerebral cells, cerebellar cells, neuronal trauma cells, glial scar cells, spinal cord cells, optic nerve cells, retina cells, kidney cells, and cells acting during fasciculation, guidance, growth, or myelination, comprising a step of administering to a subject in need of such treatment the compound as claimed in claim 20.

58. A polypeptide or a nucleic acid encoding said polypeptide, said polypeptide comprising a fragment of an AMIGO that binds to an AMIGO receptor, for use in the manufacture of a medicament for the treatment of diseases characterized by aberrant growth, migration, regeneration or proliferation of cells that express an AMIGO receptor.

59. A method of modulating the phosphorylation of a human epidermal growth factor receptor in cells or tissues comprising contacting said cells or tissues with the AMIGO compounds.

60. The method of claim 59, wherein said AMIGO compounds comprises AMIGO peptides encoded a nucleotide sequence selected from the group consisting of SEQ ID NO:1, SEQ ID NO:3, and SEQ ID NO:5.

61. The method of claim 59, wherein said AMIGO compounds comprise an anti-AMIGO antibody.


SEQUENCE LISTING (79 sequences)

SEQ ID NO: 1 - DNA, 1479 bp, Homo sapiens
  CDS (1)..(1479)
atgcacccccaccgtgacccgagaggcctctggctcctgctgccgtccttgtccctgctgctttttgaggtggccagagctggccgagccgtggttagctgtcctgccgcctgcttgtgcgccagcaacatcctcagctgctccaagcagcagctgcccaatgtgccccattccttgcccagttacacagcactactggacctcagtcacaacaacctgagccgcctgcgggccgagtggacccccacgcgcctgacccaactgcactccctgctgctgagccacaaccacctgaacttcatctcctctgaggccttttccccggtacccaacctgcgctacctggacctctcctccaaccagctgcgtacactggatgagttcctgttcagtgacctgcaagtactggaggtgctgctgctctacaataaccacatcatggcggtggaccggtgcgccttcgatgacatggcccagctgcagaaactctacttgagccagaaccagatctctcgcttccctctggaactggtcaaggaaggagccaagctacccaaactaacgctcctggatctctcttctaacaagctgaagaacttgccattgcctgacctgcagaagctgccggcctggatcaagaatgggctgtacctacataacaaccccctgaactgcgactgtgagctctaccagctgttttcacactggcagtatcggcagctgagctccgtgatggactttcaagaggatctgtactgcatgaactccaagaagctgcacaatgtcttcaacctgagtttcctcaactgtggcgagtacaaggagcgtgcctgggaggcccacctgggtgacaccttgatcatcaagtgtgacaccaagcagcaagggatgaccaaggtgtgggtgacaccaagtaatgaacgggtgctagatgaggtgaccaatggcacagtgagtgtgtctaaggatggcagtcttcttttccagcaggtgcaggtcgaggacggtggtgtgtatacctgctatgccatgggagagactttcaatgagacactgtctgtggaattgaaagtgcacaatttcaccttgcacggacaccatgacaccctcaacacagcctataccaccctagtgggctgtatccttagtgtggtcctggtcctcatatacctatacctcaccccttgccgctgctggtgccggggtgtagagaagccttccagccatcaaggagacagcctcagctcttccatgcttagtaccacacccaaccatgatcctatggctggtggggacaaagatgatggttttgaccggcgggtggctttcctggaacctgctggacctgggcagggtcaaaacggcaagctcaagccaggcaacaccctgccagtgcctgaggccacaggcaagggccaacggaggatgtcggatccagaatcagtcagctcggtcttctctgatacgcccattgtggtg

SEQ ID NO: 2 - protein, 493 aa, Homo sapiens
MHPHRDPRGLWLLLPSLSLLLFEVARAGRAVVSCPAACLCASNILSCSKQQLPNVPHSLPSYTALLDLSHNNLSRLRAEWTPTRLTQLHSLLLSHNHLNFISSEAFSPVPNLRYLDLSSNQLRTLDEFLFSDLQVLEVLLLYNNHIMAVDRCAFDDMAQLQKLYLSQNQISRFPLELVKEGAKLPKLTLLDLSSNKLKNLPLPDLQKLPAWIKNGLYLHNNPLNCDCELYQLFSHWQYRQLSSVMDFQEDLYCMNSKKLHNVFNLSFLNCGEYKERAWEAHLGDTLIIKCDTKQQGMTKVWVTPSNERVLDEVTNGTVSVSKDGSLLFQQVQVEDGGVYTCYAMGETFNETLSVELKVHNFTLHGHHDTLNTAYTTLVGCILSVVLVLIYLYLTPCRCWCRGVEKPSSHQGDSLSSSMLSTTPNHDPMAGGDKDDGFDRRVAFLEPAGPGQGQNGKLKPGNTLPVPEATGKGQRRMSDPESVSSVFSDTPIVV

SEQ ID NO: 3 - DNA, 1566 bp, Homo sapiens
  CDS (1)..(1566)
atgtcgttacgtgtacacactctgcccaccctgcttggagccgtcgtcagaccgggctgcagggagctgctgtgtttgctgatgatcacagtgactgtgggccctggtgcctctggggtgtgccccaccgcttgcatctgtgccactgacatcgtcagctgcaccaacaaaaacctgtccaaggtgcctgggaaccttttcagactgattaagagactggacctgagttataacagaattgggcttctggattctgagtggattccagtatcgtttgcaaagctgaacaccctaattcttcgtcataacaacatcaccagcatttccacgggcagtttttccacaactccaaatttgaagtgtcttgacttatcgtccaataagctgaagacggtgaaaaatgctgtattccaagagttgaaggttctggaagtgcttctgctttacaacaatcacatatcctatctcgatccttcagcgtttggagggctctcccagttgcagaaactctacttaagtggaaattttctcacacagtttccgatggatttgtatgttggaaggttcaagctggcagaactgatgtttttagatgtttcttataaccgaattccttccatgccaatgcaccacataaatttagtgccaggaaaacagctgagaggcatctaccttcatggaaacccatttgtctgtgactgttccctgtactccttgctggtcttttggtatcgtaggcactttagctcagtgatggattttaagaacgattacacctgtcgcctgtggtctgactccaggcactcgcgtcaggtacttctgctccaggatagctttatgaattgctctgacagcatcatcaatggttcctttcgtgcgcttggctttattcatgaggctcaggtcggggaaagactgatggtccactgtgacagcaagacaggtaatgcaaatacggatttcatctgggtgggtccagataacagactgctagagccggataaagagatggaaaacttttacgtgtttcacaatggaagtctggttatagaaagccctcgttttgaggatgctggagtgtattcttgtatcgcaatgaataagcaacgcctgttaaatgaaactgtggacgtcacaataaatgtgagcaatttcactgtaagcagatcccatgctcatgaggcatttaacacagcttttaccactcttgctgcttgcgtggccagtatcgttttggtacttttgtacctctatctgactccatgcccctgcaagtgtaaaaccaagagacagaaaaatatgctacaccaaagcaatgcccattcatcgattctcagtcctggccccgctagtgatgcctccgctgatgaacggaaggcaggtgcaggtaaaagagtggtgtttttggaacccctgaaggatactgcagcagggcagaacgggaaagtcaggctctttcccagcgaggcagtgatagctgagggcatcctaaagtccacgagggggaaatctgactcagattcagtcaattcagtgttttctgacacaccttttgtggcgtccact

SEQ ID NO: 4 - protein, 522 aa, Homo sapiens
MSLRVHTLPTLLGAVVRPGCRELLCLLMITVTVGPGASGVCPTACICATDIVSCTNKNLSKVPGNLFRLIKRLDLSYNRIGLLDSEWIPVSFAKLNTLILRHNNITSISTGSFSTTPNLKCLDLSSNKLKTVKNAVFQELKVLEVLLLYNNHISYLDPSAFGGLSQLQKLYLSGNFLTQFPMDLYVGRFKLAELMFLDVSYNRIPSMPMHHINLVPGKQLRGIYLHGNPFVCDCSLYSLLVFWYRRHFSSVMDFKNDYTCRLWSDSRHSRQVLLLQDSFMNCSDSIINGSFRALGFIHEAQVGERLMVHCDSKTGNANTDFIWVGPDNRLLEPDKEMENFYVFHNGSLVIESPRFEDAGVYSCIAMNKQRLLNETVDVTINVSNFTVSRSHAHEAFNTAFTTLAACVASIVLVLLYLYLTPCPCKCKTKRQKNMLHQSNAHSSILSPGPASDASADERKAGAGKRVVFLEPLKDTAAGQNGKVRLFPSEAVIAEGILKSTRGKSDSDSVNSVFSDTPFVAST

SEQ ID NO: 5 - DNA, 1512 bp, Homo sapiens
  CDS (1)..(1512)
atgacctggttggtgctgctggggacactgctctgcatgctgcgcgttgggttaggcaccccggactccgagggtttcccgccccgtgcgctccacaactgcccctacaaatgtatctgcgctgccgacctgctaagctgcactggcctagggctgcaggacgtgccagccgagttacctgccgctactgcggacctcgacctgagccacaacgcgctccagcgcctgcgccccggctggttggcgcccctcttccagctgcgcgccctgcacctagaccacaacgaactagatgcgctgggtcgcggcgtcttcgtcaacgccagcggcctgaggctgctcgatctatcatctaacacgttgcgggcgcttggccgccacgacctcgacgggctgggggcgctggagaagctgcttctgttcaataaccgcttggtgcacttggacgagcatgccttccacggcctgcgcgcgctcagccatctctacctgggctgcaacgaactcgcctcgttctccttcgaccacctgcacggtctgagcgccacccacctgcttactctggacctctcctccaaccggctgggacacatctccgtacctgagctggccgcgctgccggccttcctcaagaacggcctctacttgcacaacaaccctttgccttgcgactgccgcctctaccacctgctacagcgctggcaccagcggggcctgagcgccgtgcgcgactttgcgcgcgagtacgtatgcttggccttcaaggtacccgcgtcccgcgtgcgcttcttccagcacagccgcgtctttgagaactgctcgtcggccccagctcttggcctagagcggccggaagagcacctgtacgcgctggtgggtcggtccctgaggctttactgcaacaccagcgtcccggccatgcgcattgcctgggtttcgccgcagcaggagcttctcagggcgccaggatcccgcgatggcagcatcgcggtgctggccgacggcagcttggccataggcaacgtacaggagcagcatgcgggactcttcgtgtgcctggccactgggccccgcctgcaccacaaccagacgcacgagtacaacgtgagcgtgcactttccgcgcccagagcccgaggctttcaacacaggcttcaccacactgctgggctgtgccgtgggccttgtgctcgtgctgctctacctgttcgccccaccctgccgctgctgccgccgtgcctgccgctgccgccgctggccccaaacacccagcccgctccaagagctgagcgcacagtcctcagtactcagcaccacaccgccagacgcacccagccgcaaggccagcgtccacaagcacgtagtctttctggagccaggccggaggggcctcaatggccgcgtgcagctggcagtagctgaggaattcgatctctacaaccctggaggcctgcagctgaaggctggctctgagtccgccagctccataggctccgagggtcccatgacaacc

SEQ ID NO: 6 - protein, 504 aa, Homo sapiens
MTWLVLLGTLLCMLRVGLGTPDSEGFPPRALHNCPYKCICAADLLSCTGLGLQDVPAELPAATADLDLSHNALQRLRPGWLAPLFQLRALHLDHNELDALGRGVFVNASGLRLLDLSSNTLRALGRHDLDGLGALEKLLLFNNRLVHLDEHAFHGLRALSHLYLGCNELASFSFDHLHGLSATHLLTLDLSSNRLGHISVPELAALPAFLKNGLYLHNNPLPCDCRLYHLLQRWHQRGLSAVRDFAREYVCLAFKVPASRVRFFQHSRVFENCSSAPALGLERPEEHLYALVGRSLRLYCNTSVPAMRIAWVSPQQELLRAPGSRDGSIAVLADGSLAIGNVQEQHAGLFVCLATGPRLHHNQTHEYNVSVHFPRPEPEAFNTGFTTLLGCAVGLVLVLLYLFAPPCRCCRRACRCRRWPQTPSPLQELSAQSSVLSTTPPDAPSRKASVHKHVVFLEPGRRGLNGRVQLAVAEEFDLYNPGGLQLKAGSESASSIGSEGPMTT

SEQ ID NO: 7 - DNA, 1827 bp, Artificial Sequence
  Description of Artificial Sequence Ig-fusion protein
atgcaaccccagcgtgacctgcgaggcctctggctcctgctgctctccgtgttcctgcttctctttgaggtagccagggccggccgatctgtggttagttgtcccgccaactgcctgtgcgccagcaacatcctcagctgctccaagcagcagctgcccaatgtgccccaatctttgcccagctacacagcactgctggacctcagccacaacaacttgagcaggctgcgggccgagtggacccccacccggctgaccaacctgcactccctgctgctgagccacaaccacctgaacttcatctcctccgaggccttcgtccccgtacccaaccttaggtacttggacctctcctccaaccatcttcacacgctggatgagttcctgttcagcgacctgcaggcgctggaagtgctgttgctctacaataaccacattgtggtggtggaccggaatgcctttgaggacatggcccagctgcagaaactctacttaagccagaatcagatctctcgctttcctgtggaactgatcaaggatgggaacaaattacccaaactgatgctcttggatctgtcctccaacaagctgaagaagttgcccctgactgacctgcagaaattgccagcctgggtcaagaatgggctatacctgcataacaaccccttggagtgcgactgcaagctctaccagctcttttcgcactggcagtaccggcagctgagctctgtgatggacttccaggaggacctgtactgcatgcactccaagaagctgcacaacatcttcagcctggatttcttcaattgcagcgagtacaaggaaagtgcctgggaggctcacctgggagacaccttgaccatcaggtgtgacaccaaacagcaaggcatgaccaaagtgtgggtgtccccaagcaatgaacaggtgctaagtcaggggtccaatggctcggtgagcgtgaggaatggcgaccttttttttaaaaaggtgcaggtcgaggatgggggtgtgtatacctgttacgccatgggggagactttcaacgagacactgtctgtggagttgaaagtgtataacttcaccttgcacggacaccatgacaccctcaacggatccgaggtgctgttccagggccccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 8 - protein, 609 aa, Artificial Sequence
  Description of Artificial Sequence Ig-fusion protein
MQPQRDLRGLWLLLLSVFLLLFEVARAGRSVVSCPANCLCASNILSCSKQQLPNVPQSLPSYTALLDLSHNNLSRLRAEWTPTRLTNLHSLLLSHNHLNFISSEAFVPVPNLRYLDLSSNHLHTLDEFLFSDLQALEVLLLYNNHIVVVDRNAFEDMAQLQKLYLSQNQISRFPVELIKDGNKLPKLMLLDLSSNKLKKLPLTDLQKLPAWVKNGLYLHNNPLECDCKLYQLFSHWQYRQLSSVMDFQEDLYCMHSKKLHNIFSLDFFNCSEYKESAWEAHLGDTLTIRCDTKQQGMTKVWVSPSNEQVLSQGSNGSVSVRNGDLFFKKVQVEDGGVYTCYAMGETFNETLSVELKVYNFTLHGHHDTLNGSEVLFQGPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - DNA, 1920 bp, Artificial Sequence
  Description of Artificial Sequence Ig-fusion protein
atgtcgttaaggttccacacactgcccaccctgcctagagctgtcaaaccgggttgcagagagctgctgtgtctgttggtgatcgcagtgatggtgagccccagcgcctcaggaatgtgccccactgcttgcatctgtgccaccgacattgtcagctgcaccaacaaaaacctatctaaggtgcccgggaaccttttcagactgattaaaagactggatctgagctataacagaatcggactgttggatgccgactggatcccggtgtcgtttgtcaagctgagcaccttaattcttcgccacaacaacatcaccagcatctccacgggcagtttctccacaaccccaaatttaaagtgtctggacttatcatccaataggctgaagtcggtaaagagtgccacattccaagagctgaaggctctggaagtactgctgctgtacaacaaccacatttcctatctggaccccgcagcgttcggggggctttcccacttgcagaaactctatctgagtgggaactttctcacacagttccctatggatttgtatactgggaggttcaagctggctgatctgacatttttagatgtttcctataatcgaatcccttccataccgatgcaccatataaacttagtgccggggagacagctgagaggcatctaccttcacgggaacccatttgtatgtgactgttctctgtactcgttgctgatcttttggtaccgtaggcactttagctccgtgatggattttaagaatgactatacctgtcgcctgtggtctgactccaggcactcccaccagctgcagctgctccaggagagctttctgaactgttcttacagcgttatcaacggctccttccacgcacttggctttatccacgaggctcaggttggggagagggcgatcgtccactgtgacagcaagactggcaatggaaatactgatttcatctgggtcggtcccgataacaggctgctggagccagataaagacatggggaactttcgtgtgttttacaacggaagtctggtcatagagaaccctggctttgaggacgccggggtatattcttgtatcgcaatgaacaggcagcggctgttaaacgagacggtggatatcatgatcaacgtgagcaatttcaccataaacagatcccacgcccacgaggcggcggccgcggatcccatcgaaggtcgtggtggtggtggtggtgatcccaaatcttgtgacaaacctcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 10 - protein, 640 aa, Artificial Sequence
  Description of Artificial Sequence Ig-fusion protein
MSLRFHTLPTLPRAVKPGCRELLCLLVIAVMVSPSASGMCPTACICATDIVSCTNKNLSKVPGNLFRLIKRLDLSYNRIGLLDADWIPVSFVKLSTLILRHNNITSISTGSFSTTPNLKCLDLSSNRLKSVKSATFQELKALEVLLLYNNHISYLDPAAFGGLSHLQKLYLSGNFLTQFPMDLYTGRFKLADLTFLDVSYNRIPSIPMHHINLVPGRQLRGIYLHGNPFVCDCSLYSLLIFWYRRHFSSVMDFKNDYTCRLWSDSRHSHQLQLLQESFLNCSYSVINGSFHALGFIHEAQVGERAIVHCDSKTGNGNTDFIWVGPDNRLLEPDKDMGNFRVFYNGSLVIENPGFEDAGVYSCIAMNRQRLLNETVDIMINVSNFTINRSHAHEAAAADPIEGRGGGGGDPKSCDKPHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - DNA, 1887 bp, Artificial Sequence
  Description of Artificial Sequence Ig-fusion protein
atggcctggctagtgctatcaggtatactactatgcatgttgggtgctggattgggcacttcagacttggaggatgttctgcctcctgctccccacaactgccccgatatatgcatctgtgctgccgatgtgttgagctgtgcgggccgtgggttacaggacttgccggtagcactgcctaccactgctgcagaactcgatttgagccacaacgcactcaaacgcctgcacccggggtggttagcgcccctctcccggctgcgtgccttgcacctaggctataataagctggaagtcctgggccatggtgcgttcaccaatgccagtggcctgaggacacttgacctgtcctctaatatgttaaggatgctccatacccatgacctggatggcctggaggagctggagaagttacttctgttcaataacagcctgatgcacttggacctggatgccttccagggcctgcgcatgcttagccacctctatctcagctgcaacgagctctcctctttctctttcaaccacttgcacggtctggggttaacccgcctgcggactctggacctctcctccaactggctgaaacatatctccatccctgagttggctgcactgccaacttatctcaagaacaggctctacctgcacaacaacccgctgccctgtgactgcagcctctaccacctgctccggcgctggcaccagcggggcctgagtgccctgcatgattttgaacgcgagtacacatgcttggtctttaaggtgtcagagtcccgagtgcgcttttttgagcacagccgggtcttcaagaactgctctgtggctgcagctccaggcttagagctgcctgaagagcagctgcacgcgcaggtgggccagtccctgaggctcttctgcaacaccagtgtgcctgccactcgggtggcctgggtctccccgaagaatgagctgcttgtggcgccagcctctcaggatggtagcatcgctgtgttggctgatggcagcttagccataggcagggtgcaagagcagcacgcaggcgtctttgtgtgcctggccagtgggccccgcctgcaccacaaccagacacttgagtacaatgtgagtgtgcaaaaggctcgccccgagccagagactttcaacacagcggccgcggatcccatcgaaggtcgtggtggtggtggtggtgatcccaaatcttgtgacaaacctcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 12 - protein, 629 aa, Artificial Sequence
  Description of Artificial Sequence Ig-fusion protein
MAWLVLSGILLCMLGAGLGTSDLEDVLPPAPHNCPDICICAADVLSCAGRGLQDLPVALPTTAAELDLSHNALKRLHPGWLAPLSRLRALHLGYNKLEVLGHGAFTNASGLRTLDLSSNMLRMLHTHDLDGLEELEKLLLFNNSLMHLDLDAFQGLRMLSHLYLSCNELSSFSFNHLHGLGLTRLRTLDLSSNWLKHISIPELAALPTYLKNRLYLHNNPLPCDCSLYHLLRRWHQRGLSALHDFEREYTCLVFKVSESRVRFFEHSRVFKNCSVAAAPGLELPEEQLHAQVGQSLRLFCNTSVPATRVAWVSPKNELLVAPASQDGSIAVLADGSLAIGRVQEQHAGVFVCLASGPRLHHNQTLEYNVSVQKARPEPETFNTAAADPIEGRGGGGGDPKSCDKPHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - DNA, 1476 bp, Mus musculus
  CDS (1)..(1476)
atgcaaccccagcgtgacctgcgaggcctctggctcctgctgctctccgtgttcctgcttctctttgaggtagccagggccggccgatctgtggttagttgtcccgccaactgcctgtgcgccagcaacatcctcagctgctccaagcagcagctgcccaatgtgccccaatctttgcccagctacacagcactgctggacctcagccacaacaacttgagcaggctgcgggccgagtggacccccacccggctgaccaacctgcactccctgctgctgagccacaaccacctgaacttcatctcctccgaggccttcgtccccgtacccaaccttaggtacttggacctctcctccaaccatcttcacacgctggatgagttcctgttcagcgacctgcaggcgctggaagtgctgttgctctacaataaccacattgtggtggtggaccggaatgcctttgaggacatggcccagctgcagaaactctacttaagccagaatcagatctctcgctttcctgtggaactgatcaaggatgggaacaaattacccaaactgatgctcttggatctgtcctccaacaagctgaagaagttgcccctgactgacctgcagaaattgccagcctgggtcaagaatgggctatacctgcataacaaccccttggagtgcgactgcaagctctaccagctcttttcgcactggcagtaccggcagctgagctctgtgatggacttccaggaggacctgtactgcatgcactccaagaagctgcacaacatcttcagcctggatttcttcaattgcagcgagtacaaggaaagtgcctgggaggctcacctgggagacaccttgaccatcaggtgtgacaccaaacagcaaggcatgaccaaagtgtgggtgtccccaagcaatgaacaggtgctaagtcaggggtccaatggctcggtgagcgtgaggaatggcgaccttttttttaaaaaggtgcaggtcgaggatgggggtgtgtatacctgttacgccatgggggagactttcaacgagacactgtctgtggagttgaaagtgtataacttcaccttgcacggacaccatgacaccctcaacacagcctacactaccctggtgggctgtatcctcagtgtggttctggtcctcatatacttgtacctcaccccttgccgctgctggtgtcggggtgtggagaaaccttccagccaccaaggagatagcctcagctcttctatgctcagtaccacacccaaccacgaccctatggctggtggggacaaagatgatggttttgaccggcgggtggccttcctggaacctgctggacccgggcagggtcaaaatggcaaactcaagccaggcaacactctgccggtgcccgaagctacaggcaagggccaacggaggatgtccgatccagagtcggtcagctcggtcttttctgatacacccattgtggtg

SEQ ID NO: 14 - protein, 492 aa, Mus musculus
MQPQRDLRGLWLLLLSVFLLLFEVARAGRSVVSCPANCLCASNILSCSKQQLPNVPQSLPSYTALLDLSHNNLSRLRAEWTPTRLTNLHSLLLSHNHLNFISSEAFVPVPNLRYLDLSSNHLHTLDEFLFSDLQALEVLLLYNNHIVVVDRNAFEDMAQLQKLYLSQNQISRFPVELIKDGNKLPKLMLLDLSSNKLKKLPLTDLQKLPAWVKNGLYLHNNPLECDCKLYQLFSHWQYRQLSSVMDFQEDLYCMHSKKLHNIFSLDFFNCSEYKESAWEAHLGDTLTIRCDTKQQGMTKVWVSPSNEQVLSQGSNGSVSVRNGDLFFKKVQVEDGGVYTCYAMGETFNETLSVELKVYNFTLHGHHDTLNTAYTTLVGCILSVVLVLIYLYLTPCRCWCRGVEKPSSHQGDSLSSSMLSTTPNHDPMAGGDKDDGFDRRVAFLEPAGPGQGQNGKLKPGNTLPVPEATGKGQRRMSDPESVSSVFSDTPIVV

SEQ ID NO: 15 - DNA, 1557 bp, Mus musculus
  CDS (1)..(1557)
atgtcgttaaggttccacacactgcccaccctgcctagagctgtcaaaccgggttgcagagagctgctgtgtctgttggtgatcgcagtgatggtgagccccagcgcctcaggaatgtgccccactgcttgcatctgtgccaccgacattgtcagctgcaccaacaaaaacctatctaaggtgcccgggaaccttttcagactgattaaaagactggatctgagctataacagaatcggactgttggatgccgactggatcccggtgtcgtttgtcaagctgagcaccttaattcttcgccacaacaacatcaccagcatctccacgggcagtttctccacaaccccaaatttaaagtgtctggacttatcatccaataggctgaagtcggtaaagagtgccacattccaagagctgaaggctctggaagtactgctgctgtacaacaaccacatttcctatctggaccccgcagcgttcggggggctttcccacttgcagaaactctatctgagtgggaactttctcacacagttccctatggatttgtatactgggaggttcaagctggctgatctgacatttttagatgtttcctataatcgaatcccttccataccgatgcaccatataaacttagtgccggggagacagctgagaggcatctaccttcacgggaacccatttgtatgtgactgttctctgtactcgttgctgatcttttggtaccgtaggcactttagctccgtgatggattttaagaatgactatacctgtcgcctgtggtctgactccaggcactcccaccagctgcagctgctccaggagagctttctgaactgttcttacagcgttatcaacggctccttccacgcacttggctttatccacgaggctcaggttggggagagggcgatcgtccactgtgacagcaagactggcaatggaaatactgatttcatctgggtcggtcccgataacaggctgctggagccagataaagacatggggaactttcgtgtgttttacaacggaagtctggtcatagagaaccctggctttgaggacgccggggtatattcttgtatcgcaatgaacaggcagcggctgttaaacgagacggtggatatcatgatcaacgtgagcaatttcaccataaacagatcccacgcccacgaggcgtttaacacggcctttaccaccctggctgcctgcgtggccagtatagttctagtgctactgtatctgtacctgacgccgtgcccatgcaaatgcaaagccaagagacagaaaaacacgctgagccaaagcagtgcccactcgtccattctcagtcctggccccactggcgatgcctctgctgacgatcggaaggcaggtaaaagagtcgtgtttctggagcccctgaaggacacggcggccggacagaatggcaaagtcaagcttttccccagtgagaccgttatagccgagggcatcttaaagtccaccagggcaaagtctgactcagactcagtcaattccgtgttctcagacacaccctttgtggcatccact

SEQ ID NO: 16 - protein, 519 aa, Mus musculus
MSLRFHTLPTLPRAVKPGCRELLCLLVIAVMVSPSASGMCPTACICATDIVSCTNKNLSKVPGNLFRLIKRLDLSYNRIGLLDADWIPVSFVKLSTLILRHNNITSISTGSFSTTPNLKCLDLSSNRLKSVKSATFQELKALEVLLLYNNHISYLDPAAFGGLSHLQKLYLSGNFLTQFPMDLYTGRFKLADLTFLDVSYNRIPSIPMHHINLVPGRQLRGIYLHGNPFVCDCSLYSLLIFWYRRHFSSVMDFKNDYTCRLWSDSRHSHQLQLLQESFLNCSYSVINGSFHALGFIHEAQVGERAIVHCDSKTGNGNTDFIWVGPDNRLLEPDKDMGNFRVFYNGSLVIENPGFEDAGVYSCIAMNRQRLLNETVDIMINVSNFTINRSHAHEAFNTAFTTLAACVASIVLVLLYLYLTPCPCKCKAKRQKNTLSQSSAHSSILSPGPTGDASADDRKAGKRVVFLEPLKDTAAGQNGKVKLFPSETVIAEGILKSTRAKSDSDSVNSVFSDTPFVAST

SEQ ID NO: 17 - DNA, 1524 bp, Mus musculus
  CDS (1)..(1524)
atggcctggctagtgctatcaggtatactactatgcatgttgggtgctggattgggcacttcagacttggaggatgttctgcctcctgctccccacaactgccccgatatatgcatctgtgctgccgatgtgttgagctgtgcgggccgtgggttacaggacttgccggtagcactgcctaccactgctgcagaactcgatttgagccacaacgcactcaaacgcctgcacccggggtggttagcgcccctctcccggctgcgtgccttgcacctaggctataataagctggaagtcctgggccatggtgcgttcaccaatgccagtggcctgaggacacttgacctgtcctctaatatgttaaggatgctccatacccatgacctggatggcctggaggagctggagaagttacttctgttcaataacagcctgatgcacttggacctggatgccttccagggcctgcgcatgcttagccacctctatctcagctgcaacgagctctcctctttctctttcaaccacttgcacggtctggggttaacccgcctgcggactctggacctctcctccaactggctgaaacatatctccatccctgagttggctgcactgccaacttatctcaagaacaggctctacctgcacaacaacccgctgccctgtgactgcagcctctaccacctgctccggcgctggcaccagcggggcctgagtgccctgcatgattttgaacgcgagtacacatgcttggtctttaaggtgtcagagtcccgagtgcgcttttttgagcacagccgggtcttcaagaactgctctgtggctgcagctccaggcttagagctgcctgaagagcagctgcacgcgcaggtgggccagtccctgaggctcttctgcaacaccagtgtgcctgccactcgggtggcctgggtctccccgaagaatgagctgcttgtggcgccagcctctcaggatggtagcatcgctgtgttggctgatggcagcttagccataggcagggtgcaagagcagcacgcaggcgtctttgtgtgcctggccagtgggccccgcctgcaccacaaccagacacttgagtacaatgtgagtgtgcaaaaggctcgccccgagccagagactttcaacacaggctttaccaccctgctgggctgtattgtgggcctggtgctggtgttgctctacttgtttgcaccaccctgtcgtggctgctgtcactgctgtcagcgggcctgccgcaaccgttgctggccccgggcatccagtccactccaggagctgagcgcacagtcctccatgctcagcactacgccaccagatgcacccagccgcaaggccagtgtccacaagcatgtggtcttcctggagccgggcaagaagggcctcaatggccgtgtgcagctcgcagtagctgaagacttcgatctgtgcaaccccatgggcttgcaactcaaggctggctctgaatcagccagttccacgggctcagagggtctcgtgatgagc

SEQ ID NO: 18 - protein, 508 aa, Mus musculus
MAWLVLSGILLCMLGAGLGTSDLEDVLPPAPHNCPDICICAADVLSCAGRGLQDLPVALPTTAAELDLSHNALKRLHPGWLAPLSRLRALHLGYNKLEVLGHGAFTNASGLRTLDLSSNMLRMLHTHDLDGLEELEKLLLFNNSLMHLDLDAFQGLRMLSHLYLSCNELSSFSFNHLHGLGLTRLRTLDLSSNWLKHISIPELAALPTYLKNRLYLHNNPLPCDCSLYHLLRRWHQRGLSALHDFEREYTCLVFKVSESRVRFFEHSRVFKNCSVAAAPGLELPEEQLHAQVGQSLRLFCNTSVPATRVAWVSPKNELLVAPASQDGSIAVLADGSLAIGRVQEQHAGVFVCLASGPRLHHNQTLEYNVSVQKARPEPETFNTGFTTLLGCIVGLVLVLLYLFAPPCRGCCHCCQRACRNRCWPRASSPLQELSAQSSMLSTTPPDAPSRKASVHKHVVFLEPGKKGLNGRVQLAVAEDFDLCNPMGLQLKAGSESASSTGSEGLVMS

SEQ ID NO: 19 - DNA, 3630 bp, Homo sapiens
  CDS (1)..(3630)
atgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgaccgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctgaatacataaaccagtccgttcccaaaaggcccgctggctctgtgcagaatcctgtctatcacaatcagcctctgaaccccgcgcccagcagagacccacactaccaggacccccacagcactgcagtgggcaaccccgagtatctcaacactgtccagcccacctgtgtcaacagcacattcgacagccctgcccactgggcccagaaaggcagccaccaaattagcctggacaaccctgactaccagcaggacttctttcccaaggaagccaagccaaatggcatctttaagggctccacagctgaaaatgcagaatacctaagggtcgcgccacaaagcagtgaatttattggagca

SEQ ID NO: 20 - protein, 1210 aa, Homo sapiens [sequence omitted]

SEQ ID NO: 21 - DNA, 5512 bp, Homo sapiens
  CDS (167)..(3799)
  misc_feature: Human EGFR
gagctagccccggcggccgccgccgcccagaccggacgacaggccacctcgtcggcgtccgcccgagtccccgcctcgccgccaacgccacaaccaccgcgcacggccccctgactccgtccagtattgatcgggagagccggagcgagctcttcggggagcagcgatgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgaccgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctgaatacataaaccagtccgttcccaaaaggcccgctggctctgtgcagaatcctgtctatcacaatcagcctctgaaccccgcgcccagcagagacccacactaccaggacccccacagcactgcagtgggcaaccccgagtatctcaacactgtccagcccacctgtgtcaacagcacattcgacagccctgcccactgggcccagaaaggcagccaccaaattagcctggacaaccctgactaccagcaggacttctttcccaaggaagccaagccaaatggcatctttaagggctccacagctgaaaatgcagaatacctaagggtcgcgccacaaagcagtgaatttattggagcatgaccacggaggatagtatgagccctaaaaatccagactctttcgatacccaggaccaagccacagcaggtcctccatcccaacagccatgcccgcattagctcttagacccacagactggttttgcaacgtttacaccgactagccaggaagtacttccacctcgggcacattttgggaagttgcattcctttgtcttcaaactgtgaagcatttacagaaacgcatccagcaagaatattgtccctttgagcagaaatttatctttcaaagaggtatatttgaaaaaaaaaaaaaaagtatatgtgaggatttttattgattggggatcttggagtttttcattgtcgctattgatttttacttcaatgggctcttccaacaaggaagaagcttgctggtagcacttgctaccctgagttcatccaggcccaactgtgagcaaggagcacaagccacaagtcttccagaggatgcttgattccagtggttctgcttcaaggcttccactgcaaaacactaaagatccaagaaggccttcatggccccagcaggccggatcggtactgtatcaagtcatggcaggtacagtaggataagccactctgtcccttcctgggcaaagaagaaacggaggggatgaattcttccttagacttacttttgtaaaaatgtccccacggtacttactccccactgatggaccagtggtttccagtcatgagcgttagactgacttgtttgtcttccattccattgttttgaaactcagtatgccgcccctgtcttgctgtcatgaaatcagcaagagaggatgacacatcaaataataactcggattccagcccacattggattcatcagcatttggaccaatagcccacagctgagaatgtggaatacctaaggataacaccgcttttgttctcgcaaaaacgtatctcctaatttgaggctcagatgaaatgcatcaggtcctttggggcatagatcagaagactacaaaaatgaagctgctctgaaatctcctttagccatcaccccaaccccccaaaattagtttgtgttacttatggaagatagttttctccttttacttcacttcaaaagctttttactcaaagagtatatgttccctccaggtcagctgcccccaaaccccctccttacgctttgtcacacaaaaagtgtctctgccttgagtcatctattcaagcacttacagctctggccacaacagggcattttacaggtgcgaatgacagtagcattatgagtagtgtgaattcaggtagtaaatatgaaactagggtttgaaattgataatgctttcacaacatttgcagatgttttagaaggaaaaaagttccttcctaaaataatttctctacaattggaagattggaagattcagctagttaggagcccattttttcctaatctgtgtgtgccctgtaacctgactggttaacagcagtcctttgtaaacagtgttttaaactctcctagtcaatatccaccccatccaatttatcaaggaagaaatggttcagaaaatattttcagcctacagttatgttcagtcacacacacatacaaaatgttccttttgcttttaaagtaatttttgactcccagatcagtcagagcccctacagcattgttaagaaagtatttgatttttgtctcaatgaaaataaaactatattcatttcc

SEQ ID NO: 22 - protein, 1210 aa, Homo sapiens [sequence omitted]

SEQ ID NO: 23 - DNA, 5935 bp, Mus musculus
  CDS (224)..(3856)
  misc_feature: Murine EGFR
gaattcgggccctcctcttcttcccgcactgtgcgctcctcctgggctagggcgtctggatcgagtcccggagctaccgcctcccagacagacgacgggtcacctggacgcgagcctgtgtccgggtctcgtcgttgccggcgcagtcactgggcacaaccgtgggactccgtctgtctcggattaatcccggagagccagagccaacctctcccggtcagagatgcgaccctcagggaccgcgagaaccacactgctggtgctgctgaccgcgctctgcgccgcaggtggggcgttggaggaaaagaaagtctgccaaggcacaagtaacaggctcacccaactgggcacttttgaagaccactttctgagcctgcagaggatgtacaacaactgtgaagtggtccttgggaacttggaaattacctatgtgcaaaggaattacgacctttccttcttaaagaccatccaggaggtggccggctatgtcctcattgccctcaacaccgtggagagaatccctttggagaacctgcagatcatcaggggaaatgctctttatgaaaacacctatgccttagccatcctgtccaactatgggacaaacagaactgggcttagggaactgcccatgcggaacttacaggaaatcctgattggtgctgtgcgattcagcaacaaccccatcctctgcaatatggatactatccagtggagggacatcgtccaaaacgtctttatgagcaacatgtcaatggacttacagagccatccgagcagttgccccaaatgtgatccaagctgtcccaatggaagctgctggggaggaggagaggagaactgccagaaattgaccaaaatcatctgtgcccagcaatgttcccatcgctgtcgtggcaggtcccccagtgactgctgccacaaccaatgtgctgcggggtgtacagggccccgagagagtgactgtctggtctgccaaaagttccaagatgaggccacatgcaaagacacctgcccaccactcatgctgtacaaccccaccacctatcagatggatgtcaaccctgaagggaagtacagctttggtgccacctgtgtgaagaagtgcccccgaaactacgtggtgacagatcatggctcatgtgtccgagcctgtgggcctgactactacgaagtggaagaagatggcatccgcaagtgtaaaaaatgtgatgggccctgtcgcaaagtttgtaatggcataggcattggtgaatttaaagacacactctccataaatgctacaaacatcaaacacttcaaatactgcactgccatcagcggggaccttcacatcctgccagtggcctttaagggggattctttcacgcgcactcctcctctagacccacgagaactagaaattctaaaaaccgtaaaggaaataacaggctttttgctgattcaggcttggcctgataactggactgacctccatgctttcgagaacctagaaataatacgtggcagaacaaagcaacatggtcagttttctttggcggtcgttggcctgaacatcacatcactggggctgcgttccctcaaggagatcagtgatggggatgtgatcatttctggaaaccgaaatttgtgctacgcaaacacaataaactggaaaaaactcttcgggacacccaatcagaaaaccaaaatcatgaacaacagagctgagaaagactgcaaggccgtgaaccacgtctgcaatcctttatgctcctcggaaggctgctggggccctgagcccagggactgtgtctcctgccagaatgtgagcagaggcagggagtgcgtggagaaatgcaacatcctggagggggaaccaagggagtttgtggaaaattctgaatgcatccagtgccatccagaatgtctgccccaggccatgaacatcacctgtacaggcagggggccagacaactgcatccagtgtgcccactacattgatggcccacactgtgtcaagacctgcccagctggcatcatgggagagaacaacactctggtctggaagtatgcagatgccaataatgtctgccacctatgccacgccaactgtacctatggatgtgctgggccaggtcttcaaggatgtgaagtgtggccatctgggccaaagataccatctattgccactgggattgtgggtggcctcctcttcatagtggtggtggcccttgggattggcctattcatgcgaagacgtcacattgttcgaaagcgtacactacgccgcctgcttcaagagagagagctcgtggaacctctcacacccagcggagaagctccaaaccaagcccacttgaggatattaaaggaaacagaattcaaaaagatcaaagttctgggttcgggagcatttggcacagtgtataagggtctctggatcccagaaggtgagaaagtaaaaatcccggtggccatcaaggagttaagagaagccacatctccaaaagccaacaaagaaatccttgacgaagcctatgtgatggctagtgtggacaaccctcatgtatgccgcctcctgggcatctgtctgacctccactgtccagctcattacacagctcatgccctacggttgcctcctggactacgtccgagaacacaaggacaacattggctcccagtacctcctcaactggtgtgtgcagattgcaaagggcatgaactacctggaagatcggcgtttggtgcaccgtgacttggcagccaggaatgtactggtgaagacaccacagcatgtcaagatcacagattttgggctggccaaactgcttggtgctgaagagaaagaatatcatgccgaggggggcaaagtgcctatcaagtggatggctttggaatcaattttacaccgaatttatacacaccaaagtgatgtctggagctatggtgtcactgtgtgggaactgatgacctttgggtccaagccttatgatggaatcccagcaagtgacatctcatccatcctagagaaaggagaacgccttccacagccacctatctgcaccatcgatgtctacatgatcatggtcaagtgctggatgatagatgctgatagccgcccaaagttccgagagttgattcttgaattctccaaaatggcccgagacccacagcgctaccttgttatccagggggatgaaagaatgcatttgccaagccctacagactccaacttttaccgagccctgatggatgaagaggacatggaggatgtagttgatgctgatgagtatcttaccccacagcaaggcttcttcaacagcccgtccacgtcgaggactcccctcttgagttctctgagtgcaactagcaacaattccactgtggcttgcattaatagaaatgggagctgccgtgtcaaagaagacgccttcttgcagcggtacagctccgaccccacaggtgctgtaacagaggacaacatagatgacgcattcctccctgtacctgaatatgtaaaccaatctgttcccaagaggccagcaggctctgtgcagaaccctgtctatcacaatcagcccctgcatccagctcctggaagagacctgcattatcaaaatccccacagcaatgcagtgggcaaccctgagtatctcaacactgcccagcctacctgtctcagtagtgggtttaacagccctgcactctggatccagaaaggcagtcaccaaatgagcctagacaaccctgactaccagcaggacttcttccccaaggaaaccaagccaaatggcatatttaagggccccacagctgaaaatgcagagtacctacgggtggcacctccaagcagcgagtttattggagcatgacaagaaggggcatcataccagctataaaatgtctggactttctagaatcccaggaccaactatggcagcacctccacttctggtagccatgcccacgctgtgtcaaatgtcactcagactggctttaaagcataactctgatgggctttgtcactgagccaagaagtgggcctctctcctgatgcactttgggaagttgaaggtacatcaattgatcttcgaactgtgaagattccacaaaaaaggtatccatcgagaacattgtccattggaacagaagtttgcctcatggtgaggtacatatgggaaaaaaacagacatatggagcttatgtttagggaactttgggattcttgtctttattgatttgattgatgcactcttgtagtctggtacacagagttgcctggagccaactgaccagacagttggttccaccagctctgcatcaagacacttccgtggcaagacaactaaatgtataagaagtccatggatgccctgagcaggccacacttgtacagcattaaaccatggcagatacaataggataagccactttgttacttactggggctgggagaagaggaatgacggggtagaattttccctcagacgtactttttatataaatatgtccctggcacctaacacgcgctagtttaccagtgttttctattagacttccttctatgttttctgtttcattgttttgagttgtaaatatgtgttcctgtcttcatttcatgaagtaaacaaacaaacaaaaaacccagtattaagtattatcaaagaacaaccatgattccacattcgaacccattcaaaccatcagtattgtgaccaaaagcctttaactaagaaggagtaaccatgcaaaaatccatagaggaatttaacccaaaattttagtctcagcattgtgtctgctgaggtgtgtatatgagactacgaaagtgaactactcttcaaatccactttgccttcactcctctataccctaaatctagtgtaaaccacacatggaggataacttttttttttaattttaaaagtgtttattagatatgtttttcttcctggtaaactgcagccaaacatcagttaagagccatttttgataaacactatcacaatgatctcgggatccatcctttccgatttaccaagtgatggatagacgtgaactcataaacactacccataagacaaaacaatgagtgccagacaagacatcagccaggcaccagagcacagagcaggactgggcaatctgttggagatatctagaaagttcacaaaggaaacaagattgtccactaccttgtgagatctagcagtcataaataccagggaaatggaaagtgtgtttccttacagcaccaggtcttcgatcttcctaatgctgtgaccctttaatacagtttgccatgttgtggtgacccccaaccataaaattatttttgttgctacttcataactgtaaatttgctactcttacagaccacaatgtaaatatctgatatgctatctgatatgcaggctatctgacagaggtcgcaacccgcaggttgagagccactgccttcaaggctttaatcaagagagtagtgagctgagggctttactggtaagtcaggggcaagtccaactcaatcatcctcacatactggctgctccctcaggcctgagaatgaggcttgcagcatcctctggtttcctaaccgttatccatccctgactctcatctctgaaaatagatgtcatccatgaaattaaggagtgagaatattaagcagcatttatagagctcaaaattccatgtcatcaccaggaagtgccatgttgatcacagagaacacagaggagacatatagacagggttttgctcaaaattgggatatagaatgagcctgtcaggtacctatcaggagcggtaatccgtgagagagaaccgttgcaagccactctaactgtagcaatgaaaccctagtatttttgtactttgaaatactttcttataacaaaataaagtagcaaaaaaactgttcaaaaaaaaaaaaaaaaaaacccgaattc

SEQ ID NO: 24 - protein, 1210 aa, Mus musculus
  misc_feature: Murine EGFR
MRPSGTARTTLLVLLTALCAAGGALEEKKVCQGTSNRLTQLGTFEDHFLSLQRMYNNCEVVLGNLEITYVQRNYDLSFLKTIQEVAGYVLIALNTVERIPLENLQIIRGNALYENTYALAILSNYGTNRTGLRELPMRNLQEILIGAVRFSNNPILCNMDTIQWRDIVQNVFMSNMSMDLQSHPSSCPKCDPSCPNGSCWGGGEENCQKLTKIICAQQCSHRCRGRSPSDCCHNQCAAGCTGPRESDCLVCQKFQDEATCKDTCPPLMLYNPTTYQMDVNPEGKYSFGATCVKKCPRNYVVTDHGSCVRACGPDYYEVEEDGIRKCKKCDGPCRKVCNGIGIGEFKDTLSINATNIKHFKYCTAISGDLHILPVAFKGDSFTRTPPLDPRELEILKTVKEITGFLLIQAWPDNWTDLHAFENLEIIRGRTKQHGQFSLAVVGLNITSLGLRSLKEISDGDVIISGNRNLCYANTINWKKLFGTPNQKTKIMNNRAEKDCKAVNHVCNPLCSSEGCWGPEPRDCVSCQNVSRGRECVEKCNILEGEPREFVENSECIQCHPECLPQAMNITCTGRGPDNCIQCAHYIDGPHCVKTCPAGIMGENNTLVWKYADANNVCHLCHANCTYGCAGPGLQGCEVWPSGPKIPSIATGIVGGLLFIVVVALGIGLFMRRRHIVRKRTLRRLLQERELVEPLTPSGEAPNQAHLRILKETEFKKIKVLGSGAFGTVYKGLWIPEGEKVKIPVAIKELREATSPKANKEILDEAYVMASVDNPHVCRLLGICLTSTVQLITQLMPYGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYGVTVWELMTFGSKPYDGIPASDISSILEKGERLPQPPICTIDVYMIMVKCWMIDADSRPKFRELILEFSKMARDPQRYLVIQGDERMHLPSPTDSNFYRALMDEEDMEDVVDADEYLTPQQGFFNSPSTSRTPLLSSLSATSNNSTVACINRNGSCRVKEDAFLQRYSSDPTGAVTEDNIDDAFLPVPEYVNQSVPKRPAGSVQNPVYHNQPLHPAPGRDLHYQNPHSNAVGNPEYLNTAQPTCLSSGFNSPALWIQKGSHQMSLDNPDYQQDFFPKETKPNGIFKGPTAENAEYLRVAPPSSEFIGA

SEQ ID NO: 25 - DNA, 4877 bp, Homo sapiens
  CDS (443)..(4066)
  misc_feature: Human EGF
actgttgggagaggaatcgtatctccatatttcttctttcagccccaatccaagggttgtagctggaactttccatcagttcttcctttctttttcctctctaagcctttgccttgctctgtcacagtgaagtcagccagagcagggctgttaaactctgtgaaatttgtcataagggtgtcaggtatttcttactggcttccaaagaaacatagataaagaaatctttcctgtggcttcccttggcaggctgcattcagaaggtctctcagttgaagaaagagcttggaggacaacagcacaacaggagagtaaaagatgccccagggctgaggcctccgctcaggcagccgcatctggggtcaatcatactcaccttgcccgggccatgctccagcaaaatcaagctgttttcttttgaaagttcaaactcatcaagattatgctgctcactcttatcattctgttgccagtagtttcaaaatttagttttgttagtctctcagcaccgcagcactggagctgtcctgaaggtactctcgcaggaaatgggaattctacttgtgtgggtcctgcacccttcttaattttctcccatggaaatagtatctttaggattgacacagaaggaaccaattatgagcaattggtggtggatgctggtgtctcagtgatcatggattttcattataatgagaaaagaatctattgggtggatttagaaagacaacttttgcaaagagtttttctgaatgggtcaaggcaagagagagtatgtaatatagagaaaaatgtttctggaatggcaataaattggataaatgaagaagttatttggtcaaatcaacaggaaggaatcattacagtaacagatatgaaaggaaataattcccacattcttttaagtgctttaaaatatcctgcaaatgtagcagttgatccagtagaaaggtttatattttggtcttcagaggtggctggaagcctttatagagcagatctcgatggtgtgggagtgaaggctctgttggagacatcagagaaaataacagctgtgtcattggatgtgcttgataagcggctgttttggattcagtacaacagagaaggaagcaattctcttatttgctcctgtgattatgatggaggttctgtccacattagtaaacatccaacacagcataatttgtttgcaatgtccctttttggtgaccgtatcttctattcaacatggaaaatgaagacaatttggatagccaacaaacacactggaaaggacatggttagaattaacctccattcatcatttgtaccacttggtgaactgaaagtagtgcatccacttgcacaacccaaggcagaagatgacacttgggagcctgagcagaaactttgcaaattgaggaaaggaaactgcagcagcactgtgtgtgggcaagacctccagtcacacttgtgcatgtgtgcagagggatacgccctaagtcgagaccggaagtactgtgaagatgttaatgaatgtgctttttggaatcatggctgtactcttgggtgtaaaaacacccctggatcctattactgcacgtgccctgtaggatttgttctgcttcctgatgggaaacgatgtcatcaacttgtttcctgtccacgcaatgtgtctgaatgcagccatgactgtgttctgacatcagaaggtcccttatgtttctgtcctgaaggctcagtgcttgagagagatgggaaaacatgtagcggttgttcctcacccgataatggtggatgtagccagctctgcgttcctcttagcccagtatcctgggaatgtgattgctttcctgggtatgacctacaactggatgaaaaaagctgtgcagcttcaggaccacaaccatttttgctgtttgccaattctcaagatattcgacacatgcattttgatggaacagactatggaactctgctcagccagcagatgggaatggtttatgccctagatcatgaccctgtggaaaataagatatactttgcccatacagccctgaagtggatagagagagctaatatggatggttcccagcgagaaaggcttattgaggaaggagtagatgtgccagaaggtcttgctgtggactggattggccgtagattctattggacagacagagggaaatctctgattggaaggagtgatttaaatgggaaacgttccaaaataatcactaaggagaacatctctcaaccacgaggaattgctgttcatccaatggccaagagattattctggactgatacagggattaatccacgaattgaaagttcttccctccaaggccttggccgtctggttatagccagctctgatctaatctggcccagtggaataacgattgacttcttaactgacaagttgtactggtgcgatgccaagcagtctgtgattgaaatggccaatctggatggttcaaaacgccgaagacttacccagaatgatgtaggtcacccatttgctgtagcagtgtttgaggattatgtgtggttctcagattgggctatgccatcagtaataagagtaaacaagaggactggcaaagatagagtacgtctccaaggcagcatgctgaagccctcatcactggttgtggttcatccattggcaaaaccaggagcagatccctgcttatatcaaaacggaggctgtgaacatatttgcaaaaagaggcttggaactgcttggtgttcgtgtcgtgaaggttttatgaaagcctcagatgggaaaacgtgtctggctctggatggtcatcagctgttggcaggtggtgaagttgatctaaagaaccaagtaacaccattggacatcttgtccaagactagagtgtcagaagataacattacagaatctcaacacatgctagtggctgaaatcatggtgtcagatcaagatgactgtgctcctgtgggatgcagcatgtatgctcggtgtatttcagagggagaggatgccacatgtcagtgtttgaaaggatttgctggggatggaaaactatgttctgatatagatgaatgtgagatgggtgtcccagtgtgcccccctgcctcctccaagtgcatcaacaccgaaggtggttatgtctgccggtgctcagaaggctaccaaggagatgggattcactgtcttgatattgatgagtgccaactgggggtgcacagctgtggagagaatgccagctgcacaaatacagagggaggctatacctgcatgtgtgctggacgcctgtctgaaccaggactgatttgccctgactctactccaccccctcacctcagggaagatgaccaccactattccgtaagaaatagtgactctgaatgtcccctgtcccacgatgggtactgcctccatgatggtgtgtgcatgtatattgaagcattggacaagtatgcatgcaactgtgttgttggctacatcggggagcgatgtcagtaccgagacctgaagtggtgggaactgcgccacgctggccacgggcagcagcagaaggtcatcgtggtggctgtctgcgtggtggtgcttgtcatgctgctcctcctgagcctgtggggggcccactactacaggactcagaagctgctatcgaaaaacccaaagaatccttatgaggagtcgagcagagatgtgaggagtcgcaggcctgctgacactgaggatgggatgtcctcttgccctcaaccttggtttgtggttataaaagaacaccaagacctcaagaatgggggtcaaccagtggctggtgaggatggccaggcagcagatgggtcaatgcaaccaacttcatggaggcaggagccccagttatgtggaatgggcacagagcaaggctgctggattccagtatccagtgataagggctcctgtccccaggtaatggagcgaagctttcatatgccctcctatgggacacagacccttgaagggggtgtcgagaagccccattctctcctatcagctaacccattatggcaacaaagggccctggacccaccacaccaaatggagctgactcagtgaaaactggaattaaaaggaaagtcaagaagaatgaactatgtcgatgcacagtatcttttctttcaaaagtagagcaaaactataggttttggttccacaatctctacgactaatcacctactcaatgcctggagacagatacgtagttgtgcttttgtttgctcttttaagcagtctcactgcagtcttatttccaagtaagagtactgggagaatcactaggtaacttattagaaacccaaattgggacaacagtgctttgtaaattgtgttgtcttcagcagtcaatacaaatagatttttgtttttgttgttcctgcagccccagaagaaattaggggttaaagcagacagtcacactggtttggtcagttacaaagtaatttctttgatctggacagaacatttatatcagtttcatgaaatgattggaatattacaataccgttaagatacagtgtaggcatttaactcctcattggcgtggtccatgctgatgattttgccaaaatgagttgtgatgaatcaatgaaaaatgtaatttagaaactgatttcttcagaattagatggccttattttttaaaatatttgaatgaaaacattttatttttaaaatattacacaggaggccttcggagtttcttagtcattactgtccttttcccctacagaattttccctcttggtgtgattgcacagaatttgtatgtattttcagttacaagattgtaagtaaattgcctgatttgttttcattatagacaacgatgaatttcttctaattatttaaataaaatcaccaaaaacat

SEQ ID NO: 26 - protein, 1207 aa, Homo sapiens
  misc_feature: Human EGF
MLLTLIILLPVVSKFSFVSLSAPQHWSCPEGTLAGNGNSTCVGPAPFLIFSHGNSIFRIDTEGTNYEQLVVDAGVSVIMDFHYNEKRIYWVDLERQLLQRVFLNGSRQERVCNIEKNVSGMAINWINEEVIWSNQQEGIITVTDMKGNNSHILLSALKYPANVAVDPVERFIFWSSEVAGSLYRADLDGVGVKALLETSEKITAVSLDVLDKRLFWIQYNREGSNSLICSCDYDGGSVHISKHPTQHNLFAMSLFGDRIFYSTWKMKTIWIANKHTGKDMVRINLHSSFVPLGELKVVHPLAQPKAEDDTWEPEQKLCKLRKGNCSSTVCGQDLQSHLCMCAEGYALSRDRKYCEDVNECAFWNHGCTLGCKNTPGSYYCTCPVGFVLLPDGKRCHQLVSCPRNVSECSHDCVLTSEGPLCFCPEGSVLERDGKTCSGCSSPDNGGCSQLCVPLSPVSWECDCFPGYDLQLDEKSCAASGPQPFLLFANSQDIRHMHFDGTDYGTLLSQQMGMVYALDHDPVENKIYFAHTALKWIERANMDGSQRERLIEEGVDVPEGLAVDWIGRRFYWTDRGKSLIGRSDLNGKRSKIITKENISQPRGIAVHPMAKRLFWTDTGINPRIESSSLQGLGRLVIASSDLIWPSGITIDFLTDKLYWCDAKQSVIEMANLDGSKRRRLTQNDVGHPFAVAVFEDYVWFSDWAMPSVIRVNKRTGKDRVRLQGSMLKPSSLVVVHPLAKPGADPCLYQNGGCEHICKKRLGTAWCSCREGFMKASDGKTCLALDGHQLLAGGEVDLKNQVTPLDILSKTRVSEDNITESQHMLVAEIMVSDQDDCAPVGCSMYARCISEGEDATCQCLKGFAGDGKLCSDIDECEMGVPVCPPASSKCINTEGGYVCRCSEGYQGDGIHCLDIDECQLGVHSCGENASCTNTEGGYTCMCAGRLSEPGLICPDSTPPPHLREDDHHYSVRNSDSECPLSHDGYCLHDGVCMYIEALDKYACNCVVGYIGERCQYRDLKWWELRHAGHGQQQKVIVVAVCVVVLVMLLLLSLWGAHYYRTQKLLSKNPKNPYEESSRDVRSRRPADTEDGMSSCPQPWFVVIKEHQDLKNGGQPVAGEDGQAADGSMQPTSWRQEPQLCGMGTEQGCWIPVSSDKGSCPQVMERSFHMPSYGTQTLEGGVEKPHSLLSANPLWQQRALDPPHQMELTQ

SEQ ID NO: 27 - DNA, 4749 bp, Mus musculus
  CDS (354)..(4007)
  misc_feature: Murine EGF
aaaaaaggagaagggattcctatctgtatatagggaaggaatcctatctgcatatttcgttgttagcaccatccctcatcccggtgggcttggaactttccatcaattctttcctgtctcgtttctctttcatcctttgcctggttgtgcctgtctcagggagaaatcagtcacctgcaggccttgcagggctcttaggctctgggaaatttgtcatacgggtgtcaggtacttcttattgctgtccaaagggaaaaaaaaagtgagacaaagaactctcccggagcctttccggctgcactcagaggctctcgagaggtgcagaaggacctggaaaggcagctaaataaaagatgccctggggccgaaggccaacctggctgttgctcgccttcctgctggtgtttttaaagattagcatactcagcgtcacagcatggcagaccgggaactgtcagccaggtcctctcgagagaagcgagagaagcgggacttgtgccggtcctgcccccttcctagttttctcacaaggaaagagcatctctcggattgacccagatggaacaaatcaccagcaattggtggtggatgctggcatctcagcagacatggatattcattataaaaaagagagactctattgggtggatgtagaaagacaagttttgctaagagttttccttaacgggacaggactagagaaagtgtgcaatgtagagaggaaggtgtctgggctggccatagactggatagatgatgaagttctctgggtagaccaacagaacggagtcatcaccgtaacagatatgacagggaaaaattcccgagttcttctaagttccttaaaacatccgtcaaatatagcagtggatccaatagagaggttgatgttttggtcttcagaggtgaccggcagccttcacagagcacacctcaaaggtgttgatgtaaaaacactgctggagacagggggaatatcggtgctgactctggatgtcctggacaaacggctcttctgggttcaggacagtggcgaaggaagccacgcttacattcattcctgtgattatgagggtggctccgtccgtcttatcaggcatcaagcacggcacagtttgtcttcaatggccttttttggtgatcggatcttctactcagtgttgaaaagcaaggcgatttggatagccaacaaacacacggggaaggacacggtcaggattaacctccatccatcctttgtgacacctggaaaactgatggtagtacaccctcgtgcacagcccaggacagaggacgctgctaaggatcctgaccccgaacttctcaaacagaggggaagaccatgccgcttcggtctctgtgagcgagaccccaagtcccactcgagcgcatgcgctgagggctacacgttaagccgagaccggaagtactgcgaagatgtcaatgaatgtgccactcagaatcacggctgtactcttgggtgtgaaaacacccctggatcctatcactgcacatgccccacaggatttgttctgcttcctgatgggaaacaatgtcacgaacttgtttcctgcccaggcaacgtatcaaagtgcagtcatggctgtgtcctgacatcagatggtccccggtgcatctgtcctgcaggttcagtgcttgggagagatgggaagacttgcactggttgttcatcgcctgacaatggtggatgcagccagatctgtcttcctctcaggccaggatcctgggaatgtgattgctttcctgggtatgacctacagtcagaccgaaagagctgtgcagcttcaggaccacagccacttttactgtttgcaaattcccaggacatccgacacatgcattttgatggaacagactacaaagttctgctcagccggcagatgggaatggtttttgccttggattatgaccctgtggaaagcaagatatattttgcacagacagccctgaagtggatagagagggctaatatggatgggtcccagcgagaaagactgatcacagaaggagtagatacgcttgaaggtcttgccctggactggattggccggagaatctactggacagacagtgggaagtctgttgttggagggagcgatctgagcgggaagcatcatcgaataatcatccaggagagaatctcgaggccgcgaggaatagctgtgcatccaagggccaggagactgttctggacggacgtagggatgtctccacggattgaaagcgcttcccttcaaggttccgaccgggtgctgatagccagctccaatctactggaacccagtggaatcacgattgactacttaacagacactttgtactggtgtgacaccaagaggtctgtgattgaaatggccaatctggatggctccaaacgccgaagacttatccagaacgacgtaggtcaccccttctctctagccgtgtttgaggatcacctgtgggtctcggattgggctatcccatcggtaataagggtgaacaagaggactggccaaaacagggtacgtcttcaaggcagcatgctgaagccctcgtcactggttgtggtccatccattggcaaaaccaggtgcagatccctgcttatacaggaatggaggctgtgaacacatctgccaagagagcctgggcacagctcggtgtttgtgtcgtgaaggttttgtgaaggcctgggatgggaaaatgtgtctccctcaggattatccaatcctgtcaggtgaaaatgctgatcttagtaaagaggtgacatcactgagcaactccactcaggctgaagtaccagacgatgatgggacagaatcttccacactagtggctgaaatcatggtgtcaggcatgaactatgaagatgactgtggtcccggggggtgtggaagccatgctcgatgcgtttcagacggagagactgctgagtgtcagtgtctgaaagggtttgccagggatggaaacctgtgttctgatatagatgagtgtgtgctggctagatcggactgccccagcacctcgtccaggtgcatcaacactgaaggtggctacgtctgcagatgctcagaaggctacgaaggagacgggatctcctgtttcgatattgacgagtgccagcggggggcgcacaactgcgctgagaatgccgcctgcaccaacacggagggaggctacaactgcacctgcgcaggccgcccatcctcgcccggacggagttgccctgactctaccgcaccctctctccttggggaagatggccaccatttggaccgaaatagttatccaggatgcccatcctcatatgatggatactgcctcaatggtggcgtgtgcatgcatattgaatcactggacagctacacatgcaactgtgttattggctattctggggatcgatgtcagactcgagacctacgatggtgggagctgcgtcatgctggctacgggcagaagcatgacatcatggtggtggctgtctgcatggtggcactggtcctgctgctcctcttggggatgtgggggacttactactacaggactcggaagcagctatcaaaccccccaaagaacccttgtgatgagccaagcggaagtgtgagcagcagcgggcccgacagcagcagcggggcagctgtggcttcttgtccccaaccttggtttgtggtcctagagaaacaccaagaccccaagaatgggagtctgcctgcggatggtacgaatggtgcagtagtagatgctggcctgtctccctccctgcagctcgggtcagtgcatctgacttcatggagacagaagccccacatagatggaatgggcacagggcaaagctgctggattccaccatcaagtgacagaggaccccaggaaatagagggaaactcccacctaccctcctacagacctgtggggccggagaagctgcattctctccagtcagctaatggatcgtgtcacgaaagggctccagacctgccacggcagacagagccagttaagtagaaactgggagtagacagaaggtacagaagggaaaataacaaaccaggctgatgatggtagagtgctacagacttggtactccagtttccacggctaatcactgctcgctcagggtcctgaagatagctgcacagctgcagagctgcacagcgggatagctgcgacttttgcttcttgctttaagcagttccactgaagatactcaaaagagaagtggagaaaatcattagaaaccaaagtcaagacattcatatataagctgtgtcttcttcactggacggtttgcctcttttccttttgcctcagaaggagtgggttaaagcaggtgaccccatgctctgtcaacccctgaataaatgatgtgatctacatagaagtcttagctcactctcaggaacgcttggaacactataacttttgctatgatatactgccaagtgtggcccatgctcataattgtgccttctgaattgtgataaattagtgaaaaaactgtaacttagaatctgatttattcaggattagatcatctttttatactataaaaatcttcgaatgaaaatatttaactttaaaaacattaccttaatcattgtcttttcttcttgaagtctttcccagtgaaaacgctcaattctgctgtttccatagaatttttaatttattttaagacatgagattgtaaacaaattgcttgatttattttatcctaattatttaaataaaatcaccctaaagcatc

SEQ ID NO: 28 - protein, 1217 aa, Mus musculus
  misc_feature: Murine EGF
MPWGRRPTWLLLAFLLVFLKISILSVTAWQTGNCQPGPLERSERSGTCAGPAPFLVFSQGKSISRIDPDGTNHQQLVVDAGISADMDIHYKKERLYWVDVERQVLLRVFLNGTGLEKVCNVERKVSGLAIDWIDDEVLWVDQQNGVITVTDMTGKNSRVLLSSLKHPSNIAVDPIERLMFWSSEVTGSLHRAHLKGVDVKTLLETGGISVLTLDVLDKRLFWVQDSGEGSHAYIHSCDYEGGSVRLIRHQARHSLSSMAFFGDRIFYSVLKSKAIWIANKHTGKDTVRINLHPSFVTPGKLMVVHPRAQPRTEDAAKDPDPELLKQRGRPCRFGLCERDPKSHSSACAEGYTLSRDRKYCEDVNECATQNHGCTLGCENTPGSYHCTCPTGFVLLPDGKQCHELVSCPGNVSKCSHGCVLTSDGPRCICPAGSVLGRDGKTCTGCSSPDNGGCSQICLPLRPGSWECDCFPGYDLQSDRKSCAASGPQPLLLFANSQDIRHMHFDGTDYKVLLSRQMGMVFALDYDPVESKIYFAQTALKWIERANMDGSQRERLITEGVDTLEGLALDWIGRRIYWTDSGKSVVGGSDLSGKHHRIIIQERISRPRGIAVHPRARRLFWTDVGMSPRIESASLQGSDRVLIASSNLLEPSGITIDYLTDTLYWCDTKRSVIEMANLDGSKRRRLIQNDVGHPFSLAVFEDHLWVSDWAIPSVIRVNKRTGQNRVRLQGSMLKPSSLVVVHPLAKPGADPCLYRNGGCEHICQESLGTARCLCREGFVKAWDGKMCLPQDYPILSGENADLSKEVTSLSNSTQAEVPDDDGTESSTLVAEIMVSGMNYEDDCGPGGCGSHARCVSDGETAECQCLKGFARDGNLCSDIDECVLARSDCPSTSSRCINTEGGYVCRCSEGYEGDGISCFDIDECQRGAHNCAENAACTNTEGGYNCTCAGRPSSPGRSCPDSTAPSLLGEDGHHLDRNSYPGCPSSYDGYCLNGGVCMHIESLDSYTCNCVIGYSGDRCQTRDLRWWELRHAGYGQKHDIMVVAVCMVALVLLLLLGMWGTYYYRTRKQLSNPPKNPCDEPSGSVSSSGPDSSSGAAVASCPQPWFVVLEKHQDPKNGSLPADGTNGAVVDAGLSPSLQLGSVHLTSWRQKPHIDGMGTGQSCWIPPSSDRGPQEIEGNSHLPSYRPVGPEKLHSLQSANGSCHERAPDLPRQTEPVK

SEQ ID NO: 29 - DNA, 4119 bp, Homo sapiens
  CDS (32)..(514)
  misc_feature: Human TGF- alpha
ctggagagcctgctgcccgcccgcccgtaaaatggtcccctcggctggacagctcgccctgttcgctctgggtattgtgttggctgcgtgccaggccttggagaacagcacgtccccgctgagtgcagacccgcccgtggctgcagcagtggtgtcccattttaatgactgcccagattcccacactcagttctgcttccatggaacctgcaggtttttggtgcaggaggacaagccagcatgtgtctgccattctgggtacgttggtgcacgctgtgagcatgcggacctcctggccgtggtggctgccagccagaagaagcaggccatcaccgccttggtggtggtctccatcgtggccctggctgtccttatcatcacatgtgtgctgatacactgctgccaggtccgaaaacactgtgagtggtgccgggccctcatctgccggcacgagaagcccagcgccctcctgaagggaagaaccgcttgctgccactcagaaacagtggtctgaagagcccagaggaggagtttggccaggtggactgtggcagatcaataaagaaaggcttcttcaggacagcactgccagagatgcctgggtgtgccacagaccttcctacttggcctgtaatcacctgtgcagccttttgtgggccttcaaaactctgtcaagaactccgtctgcttggggttattcagtgtgacctagagaagaaatcagcggaccacgatttcaagacttgttaaaaaagaactgcaaagagacggactcctgttcacctaggtgaggtgtgtgcagcagttggtgtctgagtccacatgtgtgcagttgtcttctgccagccatggattccaggctatatatttctttttaatgggccacctccccacaacagaattctgcccaacacaggagatttctatagttattgttttctgtcatttgcctactggggaagaaagtgaaggaggggaaactgtttaatatcacatgaagaccctagctttaagagaagctgtatcctctaaccacgagactctcaaccagcccaacatcttccatggacacatgacattgaagaccatcccaagctatcgccacccttggagatgatgtcttatttattagatggataatggttttatttttaatctcttaagtcaatgtaaaaagtataaaaccccttcagacttctacattaatgatgtatgtgttgctgactgaaaagctatactgattagaaatgtctggcctcttcaagacagctaaggcttgggaaaagtcttccagggtgcggagatggaaccagaggctgggttactggtaggaataaaggtaggggttcagaaatggtgccattgaagccacaaagccggtaaatgcctcaatacgttctgggagaaaacttagcaaatccatcagcagggatctgtcccctctgttggggagagaggaagagtgtgtgtgtctacacaggataaacccaatacatattgtactgctcagtgattaaatgggttcacttcctcgtgagccctcggtaagtatgtttagaaatagaacattagccacgagccataggcatttcaggccaaatccatgaaagggggaccagtcatttattttccattttgttgcttggttggtttgttgctttatttttaaaaggagaagtttaactttgctatttattttcgagcactaggaaaactattccagtaatttttttttcctcatttccattcaggatgccggctttattaacaaaaactctaacaagtcacctccactatgtgggtcttcctttcccctcaagagaaggagcaattgttcccctgacatctgggtccatctgacccatggggcctgcctgtgagaaacagtgggtcccttcaaatacatagtggatagctcatccctaggaattttcattaaaatttggaaacagagtaatgaagaaataatatataaactccttatgtgaggaaatgctactaatatctgaaaagtgaaagatttctatgtattaactcttaagtgcacctagcttattacatcgtgaaaggtacatttaaaatatgttaaattggcttgaaattttcagagaattttgtcttcccctaattcttcttccttggtctggaagaacaatttctatgaattttctctttattttttttttataattcagacaattctatgacccgtgtcttcatttttggcactcttatttaacaatgccacacctgaagcacttggatctgttcagagctgaccccctagcaacgtagttgacacagctccaggtttttaaattactaaaataagttcaagtttacatcccttgggccagatatgtgggttgaggcttgactgtagcatcctgcttagagaccaatcaatggacactggtttttagacctctatcaatcagtagttagcatccaagagactttgcagaggcgtaggaatgaggctggacagatggcggaacgagaggttccctgcgaagacttgagatttagtgtctgtgaatgttctagttcctaggtccagcaagtcacacctgccagtgccctcatccttatgcctgtaacacacatgcagtgagaggcctcacatatacgcctccctagaagtgccttccaagtcagtcctttggaaaccagcaggtctgaaaaagaggctgcatcaatgcaagcctggttggaccattgtccatgcctcaggatagaacagcctggcttatttggggatttttcttctagaaatcaaatgactgataagcattggctccctctgccatttaatggcaatggtagtctttggttagctgcaaaaatactccatttcaagttaaaaatgcatcttctaatccatctctgcaagctccctgtgtttccttgccctttagaaaatgaattgttcactacaattagagaatcatttaacatcctgacctggtaagctgccacacacctggcagtggggagcatcgctgtttccaatggctcaggagacaatgaaaagcccccatttaaaaaaataacaaacattttttaaaaggcctccaatactcttatggagcctggatttttcccactgctctacaggctgtgactttttttaagcatcctgacaggaaatgttttcttctacatggaaagatagacagcagccaaccctgatctggaagacagggccccggctggacacacgtggaaccaagccagggatgggctggccattgtgtccccgcaggagagatgggcagaatggccctagagttcttttccctgagaaaggagaaaaagatgggattgccactcacccacccacactggtaagggaggagaatttgtgcttctggagcttctcaagggattgtgttttgcaggtacagaaaactgcctgttatcttcaagccaggttttcgagggcacatgggtcaccagttgctttttcagtcaatttggccgggatggactaatgaggctctaacactgctcaggagacccctgccctctagttggttctgggctttgatctcttccaacctgcccagtcacagaaggaggaatgactcaaatgcccaaaaccaagaacacattgcagaagtaagacaaacatgtatatttttaaatgttctaacataagacctgttctctctagccattgatttaccaggctttctgaaagatctagtggttcacacagagagagagagagtactgaaaaagcaactcctcttcttagtcttaataatttactaaaatggtcaacttttcattatctttattataataaacctgatgcttttttttagaactccttactctgatgtctgtatatgttgcactgaaaaggttaatatttaatgttttaatttattttgtgtggtaagttaattttgatttctgtaatgtgttaatgtgattagcagttattttccttaatatctgaattatacttaaagagtagtgagcaatataagacgcaattgtgtttttcagtaatgtgcattgttattgagttgtactgtaccttatttggaaggatgaaggaatgaacctttttttcctaaaaaaaaaaaaaaaaaaaaaaaaaaaaaaaaa

SEQ ID NO: 30 - protein, 160 aa, Homo sapiens
  misc_feature: Human TGF- alpha
MVPSAGQLALFALGIVLAACQALENSTSPLSADPPVAAAVVSHFNDCPDSHTQFCFHGTCRFLVQEDKPACVCHSGYVGARCEHADLLAVVAASQKKQAITALVVVSIVALAVLIITCVLIHCCQVRKHCEWCRALICRHEKPSALLKGRTACCHSETVV

SEQ ID NO: 31 - DNA, 3776 bp, Mus musculus
  CDS (91)..(570)
  misc_feature: Murine TGF-alpha
gcgcctttttcccccgcgcacaccgcggcggcgcgcggctactcgccaaccgcagggagcgcggtggctgcagcaccctgcgctcggaagatggtccccgcgaccggacagctcgctctgctagcgctgggtatcctgttagctgtgtgccaggctctggagaacagcacatcccccctgagtgactcacccgtggcggctgcagtggtgtctcacttcaacaagtgcccagattcccacactcagtactgcttccatggaacctgccggtttttggtgcaggaagagaagccagcatgtgtctgccactctgggtacgtgggtgttcgctgtgagcatgcagacctcctggctgtggtggctgccagccagaagaagcaagccatcactgccctggtggtggtctccattgtggccctggctgtcctcattatcacctgtgtgctgatccactgctgtcagctccgcaaacactgtgagtggtgccgtgccctcgtctgcagacatgagaagcccagcgccctcctgaagggaaggactgcttgctgccactctgagacagtggtctgaagatcccagaggaggaatttggccaggtggcctatgacagcccaaccaagaaaaggcgtcttgggacaacacccctggcagtgcccaggcccatgggacatgctgggagaccttccccctcagtgcacaactgcctgggcaggcttcttccttgagagtcttcaaaactgtgtgataaagctgcctgctgggctcgctcagtacacccagagaagaggccagtggaccacattttaaagacaagttgaacacgaacctcaaagggttggccttcttgctaacccacaccgagaatgagctggggctgctgtcccctgccagccatgacttccagactgtttcttctctatgggccatctaccccccgtcccgagactccatggttggtgtacaaaatggacaaggggaaacgtctattgttctttgaagacaccatggcgtcccatgcgccctgacatctcctcaggctgtcgtcaggatgcgtgtcttatttattagatggataacatggttttatttgtaatctctttatgtcaatgtcaggcgtccgtgctagcgatgcatgagtggcgggccgtgaagttacaccaaatagaaatgtccagccactttgataaagtaaggctgcagggaggggagcctccggggggccgtagagacagaattcagatgtgcatcctgctgggaaccaacctgctatcccaaggctgtgccccctcaggcaagaaagtccctggatacatgctgtagcaaacttagcaaggccaatggcagagagtgtcccccttgtggggacaagaggacaaaagagcctgtgtgggcacctaggaaggacgcacaccccaacccttgattaagtggattagcgtcctcgtgagcccagctccagcatcagcaatgagctgtgagccaaatccatgaaggggaccagccctttattggcttttgcttatttttagaaggagaaatttgcctttcctatttattttcaagcattaagaaaaatagcccactgttttgttttgtttcccctttaataatgttggcattattaacaaaatcgctaccaagcctctccacccctctctggggaaggaaatgtgttccctgagcacccgaagatgctctgacccattgagcccacccttgttcctgggctgcttgggactgtcaggagacagaatacattcagttgcatattaaagagaccattctccaactcagaagtgaaaagtctgccccctcggccctccagggcctctgaagtaaggcatgagggaaggggtactgggcataccaagttggtttgaagcttctgtcagaagctgcctgttcctcattcttctcctttatgaatttttctcctctcatatgatctaggcattgatctgcccaggtcttaaccctttgttcacaaggcctgaagccctgaaagaagaacctgaaactgttcagtgctgtcctctaactgcttagctgacacagctccaggcagaagttgtcattcttgctattattttggtttgatgctttatcttgttttgtgagacaaggtcttgctgtgtagcccaggttgacctttaagttaagatcctcctgcttcagcaacctggaagctgggcttacagatgtgcaccaccacgcccagcactttttgattactagagaaaataggttaagtttgctttccttgggtcatacaagtgggtcaaggccaagtgtcacctgctgctcagacaccaatcaacctcactggacttcagccctctattggcccccagtgctctcagtagaccccgcctgaagtactggctcactggagggcagggagcaacacaaatggaggagatgtggggcttgttttagtttagttcacactgttagcttctgatgacaggtcttttctggtctgctggaggctcctgggtgtctgacttcttttaaagagtttgaactgatatcaaaatatcattgggaacttcctcacttaatatcccatcctgaagatgttgtcatccaagactaagcagaggtggtaaacgcttagcacgagcactcgatggtgaaggcagaggatctcgggaggacctcagctttagtgtccgaacggtcctgttctcaggtccagccagtcgcagcagccagtaccatcccagtgcctgtacacagatgcagctggctcaaacacctcccagaagtgccttcccatcagtctaccctttgcagagtcagaggctgagactaggttgagacaagtccggttggacatcgtccctgccccgacgtatgacagccctgctgcgttggtgactttgattttggaagtcagtcagacaaacgctgtgtaccttctcctgtgccatgtcacagcacagatgggcctggttagctgtaccagcgccctcctgtcagttcacactggccctctcttgatatttagtacagctacttaatcaggagcactcttagcagctaggcaggtcagagggcagtacagttgattcagtgactcaggggactagggccattcccattaaatgagctgaaattaattctagagacattgagacagctcagttagcaatggcacttaccaaaatcccagataacctgaattcaatcccctgggatcccatctggtggaaggagagaaccggcttgcccaggatgtcttctgatttctacgtacacgcacacgcacacacacacacgcacgcacacacacgcacacacacgcacatatgagtacacacaaagtctaagtttttaaaataaaaattctagaaagacctctacacctctagctataactttaaacacaccatgggacatgttttctccacggaaaagcacaattggagacccagtctgaaatgaaagatagattccaggtggacatacacagaaccaaaccatgagttagtttgtcatgtgccccagaaaaaggtgggaacaataatcccaacatccctttcctctccccccccaaaaaagggggggaggaggcatggttggtacccacccacctgcactggtgagggaagtgactatgtccctgagtcctagatggtgacgttggtcccttcaagtccccaggccaggtttcctagtgcctatgaggcactggatagaccagcaacactctgacactgcctacaggccccagtcctgtaatgagtaataaacttgtctctctcccaaggcca

SEQ ID NO: 32 - protein, 159 aa, Mus musculus
  misc_feature: Murine TGF-alpha
MVPATGQLALLALGILLAVCQALENSTSPLSDSPVAAAVVSHFNKCPDSHTQYCFHGTCRFLVQEEKPACVCHSGYVGVRCEHADLLAVVAASQKKQAITALVVVSIVALAVLIITCVLIHCCQLRKHCEWCRALVCRHEKPSALLKGRTACCHSETVV

SEQ ID NO: 33 - protein, 144 aa, Unknown
  Mammalian Slit1_LRRs1 amino acid sequence from FIG. 11
NTERLELNGNNITRIHKNDFAGLKQLRVLQLMENQIGAVERGAFDDMKELERLRLNRNQLHMLPELLFQNNQALSRLDLSENAIQAIPRKAFRGATDLKNLRLDKNQISCIEEGAFRALRGLEVLTLNNNNITTIPVSSFNHMP

SEQ ID NO: 34 - protein, 120 aa, Unknown
  Mammalian Slit1_LRRs2 amino acid sequence from FIG. 11
TMTEIRLELNGIKSIPPGAFSPYRKLRRIDLSNNQIAEIAPDAFQGLRSLNSLVLYGNKITDLPRGVFGGLYTLQLLLLNANKINCIRPDAFQDLQNLSLLSLYDNKIQSLAKGTFTSLR

SEQ ID NO: 35 - protein, 96 aa, Unknown
  Mammalian Slit1_LRRs3 amino acid sequence from FIG. 11
HLKKINLSNNKVSEIEDGAFEGAASVSELHLTANQLESIRSGMFRGLDGLRTLMLRNNRISCIHNDSFTGLRNVRLLSLYDNQITTVSPGAFDTLQ

SEQ ID NO: 36 - protein, 72 aa, Unknown
  Mammalian Slit1_LRRs4 amino acid sequence from FIG. 11
YLQLVDLSNNKISSLSNSSFTNMSQLTTLILSYNALQCIPPLAFQGLRSLRLLSLHGNDISTLQEGIFADVT

SEQ ID NO: 37 - protein, 145 aa, Unknown
  Mammalian NOGO_R amino acid sequence from FIG. 11
LLEQLDLSDNAQLRSVDPATFHGLGRLHTLHLDRCGLQELGPGLFRGLAALQYLYLQDNALQALPDDTFRDLGNLTHLFLHGNRISSVPERAFRGLHSLDRLLLHQNRVAHVHPHAFRDLGRLMTLYLFANNLSALPTEALAPLR

SEQ ID NO: 38 - protein, 148 aa, Homo sapiens
  misc_feature: AMIGO amino acid sequence from FIG. 11
YTALLDLSHNNLSRLRAEWTPTRLTQLHSLLLSHNHLNFISSEAFSPVPNLRYLDLSSNQLRTLDEFLFSDLQVLEVLLLYNNHIMAVDRCAFDDMAQLQKLYLSQNQISRFPLELVKEGAKLPKLTLLDLSSNKLKNLPLPDLQKLP

SEQ ID NO: 39 - protein, 144 aa, Artificial Sequence
  consensus sequence of the 6 LRR motifs of the AMIGO derived from multiple mammalian organisms.
XLXXLXLXXNXLXXXXXXXFXXLXXLXXLXLXXNXLXXXXXXXFXXLXXLXXLXLXXNXLXXXXXXFXXLXXLXXLXLXXNXLXXXXXXXXFXXLXXLXXLXLXXNXLXXXXXXXFXXLXXLXXLXLXXNXLXXXXXXXFXXLX

SEQ ID NO: 40 - protein, 28 aa, Unknown
  Consensus sequence of LRRs of porcine RI (mamalian)
XLEXLXLXXCXLTXXXCXXLXXXLXXXX

SEQ ID NO: 41 - protein, 29 aa, Unknown
  Consensus sequence of LRRs of porcine RI (mamalian)
XLXELXLXXNXLGDXGXXXXXXXLXXPXX

SEQ ID NO: 42 - DNA, 21 bp, Artificial Sequence
  Synthetic PCR primer
actgcttctcgcctggcccgt

SEQ ID NO: 43 - DNA, 24 bp, Artificial Sequence
  Synthetic PCR primer
gaacctccccatcagcctatactg

SEQ ID NO: 44 - DNA, 19 bp, Artificial Sequence
  Synthetic PCR primer
cagaacatgcccgggtgac

SEQ ID NO: 45 - DNA, 22 bp, Artificial Sequence
  Synthetic PCR primer
ggaccaattcccttgaggtcag

SEQ ID NO: 46 - DNA, 21 bp, Artificial Sequence
  Synthetic PCR primer
actgcttctcgcctggcccgt

SEQ ID NO: 47 - DNA, 23 bp, Artificial Sequence
  Synthetic PCR primer
aacctccccatcagcctacgctg

SEQ ID NO: 48 - DNA, 21 bp, Artificial Sequence
  Synthetic PCR primer
ctcagaggcgaccataatgtc

SEQ ID NO: 49 - DNA, 23 bp, Artificial Sequence
  Synthetic PCR primer
tgtttattttgcagaccacacac

SEQ ID NO: 50 - DNA, 21 bp, Artificial Sequence
  Synthetic PCR primer
ctcagaggcgaccataatgtc

SEQ ID NO: 51 - DNA, 21 bp, Artificial Sequence
  Synthetic PCR primer
gcgatgctgaaggctaagatg

SEQ ID NO: 52 - DNA, 25 bp, Artificial Sequence
  Synthetic PCR primer
caacctgcacagagctgctctgtac

SEQ ID NO: 53 - DNA, 25 bp, Artificial Sequence
  Synthetic PCR primer
gcacagtgcttcccaccagtatctg

SEQ ID NO: 54 - DNA, 23 bp, Artificial Sequence
  Synthetic PCR primer
agaagtaggtgagtcttggagct

SEQ ID NO: 55 - DNA, 20 bp, Artificial Sequence
  Synthetic PCR primer
tgttgtgcaggtagagcctg

SEQ ID NO: 56 - DNA, 20 bp, Artificial Sequence
  Synthetic PCR primer
agcaacatcctcagctgctc

SEQ ID NO: 57 - DNA, 21 bp, Artificial Sequence
  Synthetic PCR primer
cttcagcttgttggaggacag

SEQ ID NO: 58 - DNA, 20 bp, Artificial Sequence
  Synthetic PCR primer
ggcactttagctccgtgatg

SEQ ID NO: 59 - DNA, 20 bp, Artificial Sequence
  Synthetic PCR primer
gtctcgtttaacagccgctg

SEQ ID NO: 60 - DNA, 20 bp, Artificial Sequence
  Synthetic PCR primer
aggtgtcagagtcccgagtg

SEQ ID NO: 61 - DNA, 20 bp, Artificial Sequence
  Synthetic PCR primer
gtagagcaacaccagcacca

SEQ ID NO: 62 - DNA, 20 bp, Artificial Sequence
  Synthetic PCR primer
caacgaccccttcattgacc

SEQ ID NO: 63 - DNA, 20 bp, Artificial Sequence
  Synthetic PCR primer
agtgatggcatggactgtgg

SEQ ID NO: 64 - DNA, 28 bp, Artificial Sequence
  Synthetic PCR primer
ccgctcgagccggccgatctgtggttag

SEQ ID NO: 65 - DNA, 32 bp, Artificial Sequence
  Synthetic PCR primer
cggaattctcacaccacaatgggtctatcaga

SEQ ID NO: 66 - DNA, 31 bp, Artificial Sequence
  Synthetic PCR primer
cgggatcctagggtgactctctcccagatcc

SEQ ID NO: 67 - DNA, 29 bp, Artificial Sequence
  Synthetic PCR primer
cgggatccgttgagggtgtcatggtgtcc

SEQ ID NO: 68 - protein, 10 aa, Artificial Sequence
  Synthetic peptide derived from mouse, rat, and human.
YAMGETFNET

SEQ ID NO: 69 - DNA, 30 bp, Artificial Sequence
  Synthetic PCR primer
gcggccgctcagggcccacggtttctgcag

SEQ ID NO: 70 - DNA, 32 bp, Artificial Sequence
  Synthetic PCR primer
ggcgcgccactgggaagagvgaggaaggccac

SEQ ID NO: 71 - DNA, 37 bp, Artificial Sequence
  Synthetic PCR primer
taaactagcggccgctcatggaggctcacccatggac

SEQ ID NO: 72 - DNA, 38 bp, Artificial Sequence
  Synthetic PCR primer
agatatggcgcgccggtcgcctctgagtctcttgccag

SEQ ID NO: 73 - DNA, 30 bp, Artificial Sequence
  Synthetic PCR primer
accttaattaaccagatggcttcttctttc

SEQ ID NO: 74 - DNA, 33 bp, Artificial Sequence
  Synthetic PCR primer
agatatggcgcgccagtgactaccagggaagat

SEQ ID NO: 75 - DNA, 31 bp, Artificial Sequence; identical to SEQ ID NO: 66
  Synthetic PCR primer

SEQ ID NO: 76 - DNA, 29 bp, Artificial Sequence
  Synthetic PCR primer
cgggatccgttgagggtgtcatggtgtcc

SEQ ID NO: 77 - DNA, 39 bp, Artificial Sequence
  Synthetic PCR primer
ataagaatgcggccgccaatgtgcatcagttgtggtcag

SEQ ID NO: 78 - DNA, 31 bp, Artificial Sequence
  Synthetic PCR primer
gctctagacgtgccaagcatcctcgtgcgac

SEQ ID NO: 79 - protein, 19 aa, Artificial Sequence
  Motif of LRRs found in the AMIGO sequence. Derived from Homo Sapiens.
LXXLXLXXNXXXXXXXXXX